(19) United States
(12) Patent Application Publication
(10) Pub. No.: US 2026/0074012 A1
(43) Pub. Date: Mar. 12, 2026

(54) UNIFIED STRUCTURE MODEL FOR MOLECULE PROPERTY AND STRUCTURE PREDICTION

(71) Applicant: Isomorphic Labs Limited, London (GB)

(72) Inventors: Chia-Chun Hung, London (GB); Maxwell Elliot Jaderberg, London (GB); Joshua Patrick Bambrick, London (GB); Joss William Briody, London (GB); Kevin Michael Schaarschmidt, Cambridge (GB); Ladislav Rampášek, London (GB); Christoph Johann Feinauer, London (GB); Thomas Ayoola, London (GB); Anton Osokin, London (GB); Benedek Andras Rózemberczki, Letchworth Garden City (GB); Andrey Malinin, London (GB); Arsenii Ashukha, London (GB); Daniele Grattarola, London (GB); Joshua Simon Abramson, London (GB); Jack Tavis Dunger, London (GB); Richard Andrew Evans, London (GB); Victor Constant Bapst, London (GB); Wojciech Czarnecki, London (GB); David Reiman, Santa Cruz, CA (US); Zachary Wu, Milpitas, CA (US)

(21) Appl. No.: 19/316,998

(22) Filed: Sep. 2, 2025

Related U.S. Application Data
(60) Provisional application No. 63/692,015, filed on Sep. 6, 2024.

(51) Int. Cl. G16B 15/30; G16B 15/20; G16B 40/20

(52) U.S. Cl. CPC G16B 15/30; G16B 15/20; G16B 40/20

(57) ABSTRACT

Methods, systems, and apparatus, including computer programs encoded on a computer storage medium, for generating predictions characterizing one or more molecules. In one aspect, a method comprises obtaining molecule data characterizing a molecule; processing a network input comprising the molecule data using an embedding neural network to generate a molecule embedding representing the molecule; and processing the molecule embedding representing the molecule using a prediction machine learning model to generate an output prediction characterizing the molecule, wherein the embedding neural network has been jointly trained along with a plurality of prediction neural networks that are each configured to perform a respective prediction task by operations comprising: receiving an input molecule embedding that represents one or more molecules and that is generated by the embedding neural network; and processing the input molecule embedding to generate a corresponding prediction characterizing the one or more molecules.

### CROSS-REFERENCE TO RELATED APPLICATION

This application claims priority under 35 U.S.C. 119 to Provisional Application No. 63/692,015, filed Sep. 6, 2024, which is incorporated by reference.

### BACKGROUND

This specification relates to predicting properties and structures of molecules using machine learning models.

Machine learning models receive an input and generate an output, e.g., a predicted output, based on the received input. Some machine learning models are parametric models and generate the output based on the received input and on values of the parameters, e.g. weights, of the model.

Some machine learning models are deep models that employ multiple layers of models to generate an output for a received input. For example, a deep neural network is a deep machine learning model that includes an output layer and one or more hidden layers that each apply a non-linear transformation to a received input to generate an output.

### SUMMARY

This specification describes a system implemented as computer programs on one or more computers in one or more locations that can predict properties and structures for molecules. In particular, the described systems can predict properties and structures for molecule complexes that include multiple molecules.

For example, the described systems can receive molecule data characterizing one or more molecules (e.g., including one or more proteins, ligands, nucleic acids, etc.) and can generate, e.g., predicted physio-chemical properties, predicted joint 3-dimensional structures of molecule complexes, and so on for the one or more molecules. As a further example, the described systems can receive molecule data characterizing multiple molecules (e.g., characterizing a protein and a ligand, a first protein and a second protein, and so on) and can generate, e.g., a predicted binding affinity for the multiple molecules, a predicted joint 3-dimensional structure of a molecule complex (e.g., a protein-ligand complex, a protein-protein complex, etc.) including the multiple molecules, and so on. As another example, the described systems can receive molecule data characterizing a first molecule (e.g., a protein, a nucleic acid, etc.) and can design a second molecule (e.g., a ligand, a protein, a nucleic acid, etc.) to bind with the first molecule.

A “protein” can be understood to refer to any biological molecule that is specified by one or more sequences (or “chains”) of amino acids. For example, the term protein can refer to a protein domain, e.g., a portion of an amino acid chain of a protein that can undergo protein folding nearly independently of the rest of the protein. As another example, the term protein can refer to a protein complex, i.e., that includes multiple amino acid chains that jointly fold into a protein structure.

A “nucleic acid” can be understood to refer to any biological molecule that is specified by one or more sequences (or “chains”) of nucleotides. Examples of nucleic acids include deoxyribonucleic acid (DNA), ribonucleic acid (RNA), peptide nucleic acids, morpholino nucleic acids, locked nucleic acids, glycol nucleic acids, threose nucleic acids, and so on.

A “ligand” can refer to a molecule or compound that binds to a target molecule, e.g., a protein, a nucleic acid, and so on. Ligands can include, e.g., ions, small molecules, polymers, organic molecules, inorganic molecules, proteins, nucleic acids, biomolecules, and so forth. As used herein a small molecule can be one with a molecular weight of less than 900 daltons.

“Conditioning” a model (e.g., a generative model) or a neural network (e.g., a denoising neural network) or an operation (e.g., a self-attention operation) on conditioning data (e.g., an embedding representing a protein or a set of molecule design criteria) can refer to providing the conditioning data as an input (e.g., a side input) to the model, neural network, or operation, such that outputs generated by the model, neural network, or operation are influenced by (depend on) the conditioning data.

A “physio-chemical property” of a molecule can be any of a variety of properties characterizing chemical reactions involving the molecule. For example, physio-chemical properties of a molecule can include a solubility of the molecule, a permeability of the molecule (e.g. across a cell membrane), a chemical stability of the molecule, a lipophilicity of the molecule, a strength of plasma protein binding of the molecule, a so-called volume of distribution of the molecule (a measure of the extent to which the molecule spreads into body tissues), properties characterizing enzymatic pathways responsible for metabolizing the molecule, metabolic rate properties for the molecule, properties characterizing metabolites generated by metabolism of the molecule, and so on. A physio-chemical property of a molecule complex can be any of a variety of properties characterizing chemical reactions involving molecules of the molecule complex. For example, physio-chemical properties of a molecule complex can include a binding affinity of the molecules of the molecule complex characterizing a strength or degree of attraction between the molecules when they interact to form the molecule complex.

A 3D spatial position of an atom can be represented by a set of coordinates in an appropriate coordinate system, e.g., a 3D Cartesian coordinate system or a spherical coordinate system.

According to one aspect, there is provided a method that includes obtaining molecule data characterizing a molecule. The molecule data characterizing a molecule can be molecule data characterizing, e.g., a protein, a nucleic acid, and so forth, molecule data or characterizing a molecule complex, such as a target molecule-ligand complex, e.g. a protein-ligand complex. That is, the molecule data can characterize multiple molecules. The molecule data can, e.g. characterize a physical, e.g. 3D, or chemical structure of all or part of the molecule(s); further examples are given later.

The method involves processing a network input comprising the molecule data using an embedding neural network to generate a molecule embedding representing the molecule, and processing the molecule embedding representing the molecule using a prediction machine learning model to generate an output prediction characterizing the molecule. For example the output prediction can characterize e.g. one or more physio-chemical properties of the molecule(s), or a physical, e.g. 3D, or chemical structure of the molecule(s) or part of the molecule(s) such as a binding pocket; further examples are given later. For example the output prediction can characterize the (3D) structure or one or more properties of a protein-ligand complex. In general the output prediction characterizing the molecule can be different to the molecule data characterizing the molecule.

The embedding neural network has been jointly trained along with a plurality of prediction neural networks. Each prediction neural network is configured to perform a respective prediction task by operations comprising: receiving an input molecule embedding that represents one or more molecules and that is generated by the embedding neural network, and processing the input molecule embedding to generate a corresponding prediction characterizing the one or more molecules, such as a prediction of a physio-chemical, physical or chemical property of the molecule(s). Training the embedding neural network jointly with a prediction neural network can involve backpropagating gradients of an objective function for the prediction neural network through the prediction neural network and into the embedding neural network to update learnable parameters of the prediction neural network and the embedding neural network. The objective function can be any appropriate objective function for the prediction neural network, in general measuring a discrepancy between the prediction and a prediction target.

In some implementations the plurality of prediction neural networks comprises at least a ligand design neural network. The ligand design neural network is configured to perform a ligand design task by receiving an input molecule embedding that represents at least a portion of a molecule, and that is generated by the embedding neural network. The input molecule embedding can represent at least a portion of any target molecule for the ligand, e.g., a protein, a nucleic acid, and so forth; it can represent, as an example, part of a protein that includes a binding pocket for the ligand. The input molecule embedding is processed by the prediction neural network to generate predicted ligand data defining a predicted ligand that is predicted to bind to the protein. The predicted ligand data can be any data that defines the predicted ligand, e.g. a physical (3D) structure of the predicted ligand, such as data specifying the 3D positions of atoms of the predicted ligand; data specifying the presence and/or types of bonds between atoms of the predicted ligand; and so forth.

Particular embodiments of the subject matter described in this specification can be implemented so as to realize one or more of the following advantages.

Applications such as drug discovery can involve performing multiple prediction tasks regarding molecules and molecule complexes. As an example, designing a drug to interact with a target protein can involve designing a ligand to bind with the target protein, predicting one or more properties (e.g., a binding affinity, binding energy, etc.) of a protein-ligand complex formed by the designed ligand and the target protein, and predicting a joint 3D structure of the protein-ligand complex formed by the designed ligand and the target protein.

Conventional approaches can perform multiple prediction tasks regarding molecules and molecule complexes by maintaining a separate machine learning model for each prediction task (e.g., using separate ligand design, property prediction, and structure prediction machine learning models in the above drug discovery example). However, training and maintaining a separate machine learning model for each prediction task can be computationally inefficient, as each of the prediction machine learning models is stored in memory separately, trained separately, and used at inference separately. Many prediction tasks regarding molecules and molecule complexes, such as ligand design, property prediction, and structure prediction, share commonalities, yet conventional architectures that maintain separate machine learning models for different prediction tasks do not exploit these shared commonalities to efficiently perform multiple prediction tasks.

The systems described in this specification address these issues for performing multiple predictions for molecules and molecule complexes by using a shared embedding neural network. The shared embedding neural network can process molecule data to generate a joint molecule embedding that jointly represents one or more molecules characterized by the molecule data. The embedding neural network can be jointly trained with a plurality of prediction neural networks (e.g., including property prediction neural networks, structure prediction neural networks, ligand design neural networks, etc.) that are each configured to process joint molecule embeddings generated by the embedding neural network to perform a respective prediction task. By using a shared embedding neural network, the described system can maintain a same set of embedding neural network weights for that can be reused for multiple prediction tasks, which can increase computational efficiency for training and inference compared to conventional methods.

As a result of being jointly trained with a plurality of prediction neural networks (e.g., property prediction neural networks, structure prediction neural networks, molecule design neural networks, etc.), the embedding neural network can generate joint molecule embeddings that encode rich informational content related to the joint 3D structure of molecules. The prediction neural networks can be trained to leverage this rich informational content to generate accurate predictions for multiple molecules.

By jointly training the embedding neural network with a plurality of different prediction neural networks, the described systems can train the embedding neural network using a combined set of training data that includes training examples for each of the prediction neural networks. The embedding neural network can therefore benefit from training data for each of the plurality of prediction neural networks to generate improved joint molecule embeddings that can be used to perform a variety of prediction tasks. In turn, each of the prediction neural networks can benefit from the improved joint molecule embeddings to generate more accurate predictions for molecule complexes. The described systems can require significantly fewer computational resources (e.g., memory and computing power) to train and can generate more accurate predictions for molecule complexes compared to conventional approaches that, e.g., separately train each prediction neural network.

In addition, after training the embedding neural network, the described systems can be efficiently trained to perform additional prediction tasks. For example, to perform an additional processing task, the described systems can train a prediction machine learning model to process joint embeddings generated by the trained embedding neural network to perform the additional processing task. Training the additional prediction machine learning model using joint embeddings from a trained (e.g., fixed) embedding neural network can consume far fewer computational resources to attain a certain performance for the additional prediction task compared to conventional methods that would require training an entire separate prediction model for the additional task in isolation from already trained prediction models.

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 is a block diagram of an example molecule prediction system.

FIG. 2A a block diagram of an example embedding neural network.

FIG. 2B is a flow diagram of an example process for using an embedding neural network to generate a joint molecule embedding that jointly represents a first molecule and a second molecule.

FIG. 2C illustrates generating a joint molecule embedding representing a first molecule and a second molecule.

FIG. 3 is a flow diagram of an example process for generating a prediction for a molecule using a molecule prediction system.

FIG. 4 is a flow diagram of an example process for jointly training an embedding neural network with a prediction machine learning model.

FIG. 5A is a block diagram of an example property prediction neural network of a molecule prediction system.

FIG. 5B is a flow diagram of an example process for generating a predicted property score for a molecule complex using a graph neural network.

FIG. 5C is a flow diagram of an example process for generating a graph based on a predicted 3D structure of a molecule complex.

FIG. 5D is a flow diagram of an example process for processing a graph representing a 3D structure of a molecule complex using a graph neural network to generate a predicted property score.

FIG. 5E is a flow diagram of an example process for training a graph neural network of a property prediction neural network.

FIG. 6A is a block diagram of an example structure prediction neural network of a molecule prediction system.

FIG. 6B is a flow diagram of an example process for generating a predicted joint 3D structure for a molecule complex using a structure prediction neural network.

FIG. 7A is a block diagram of an example molecule design neural network of a molecule prediction system.

FIG. 7B is a flow diagram of an example process for generating and screening ligands for binding to a protein.

FIG. 7C illustrates generating a joint molecule embedding representing a protein and molecule design criteria using an embedding neural network.

FIG. 7D is a flow diagram of an example process for processing molecule design criteria using a design embedding neural network.

FIG. 7E provides an illustration of a collection of initial atom embeddings generated by an embedding block of a design embedding neural network.

FIG. 7F is a flow diagram of an example process for generating data defining a designed molecule based on the denoised atom state data for each atom of the designed molecule.

FIG. 8 is a flow diagram of an example process for generating bond data for a molecule generated by a molecule design neural network.

FIG. 9 is a flow diagram of an example process for in-painting and/or out-painting a protein-ligand complex.

FIG. 10A is a flow diagram of an example process for generating a predicted joint 3D structure of a molecule complex using a generative diffusion model that includes a denoising neural network.

FIG. 10B is a flow diagram of an example process for generating a denoising output using a denoising neural network conditioned on a joint molecule embedding.

FIG. 10C is a flow diagram of an example process for updating a set of current component embeddings using a self-attention operation that is implemented by a self-attention block of the denoising neural network and that is conditioned on a joint molecule embedding.

FIG. 10D is a flow diagram of an example process generating gradients of a conditioning objective function with respect to current atom state data for the atoms in a molecule complex at a current denoising time step in a sequence of denoising time steps.

FIG. 10E is a flow diagram of an example process for jointly training an embedding neural network and a generative diffusion model on a training example.

Like reference numbers and designations in the various drawings indicate like elements. Aspects and elements of the figures can be combined. For example, whilst for convenience some implementations and embodiments are described with reference to particular figures, features of implementations and embodiments described with reference to different figures may be combined in a molecule prediction system.

### DETAILED DESCRIPTION

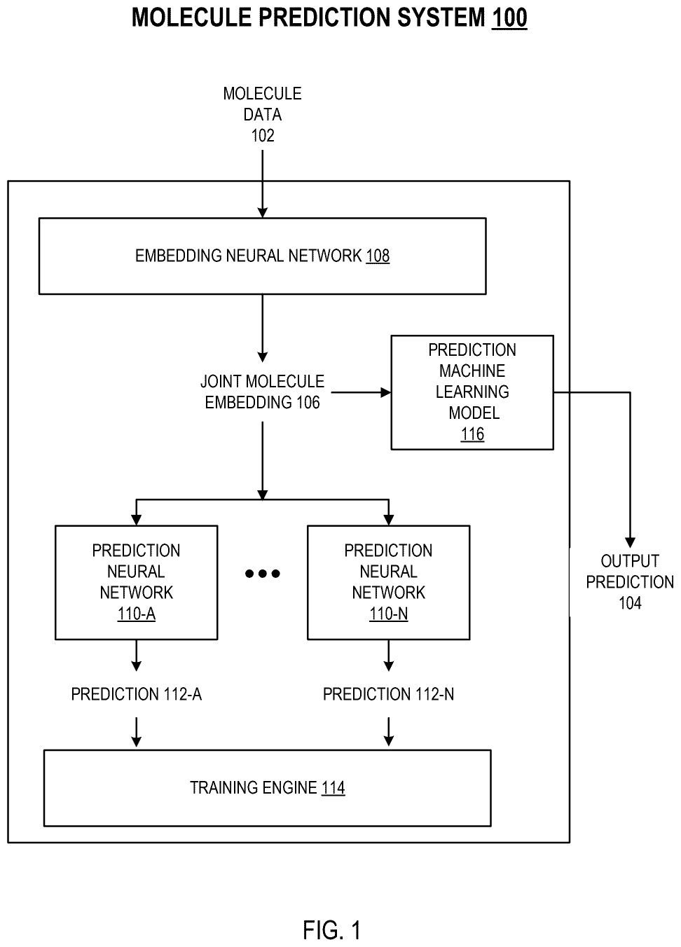

FIG. 1A shows an example molecule prediction system 100. The molecule prediction system 100 is an example of a system implemented as computer programs on one or more computers in one or more locations in which the systems, components, and techniques described below are implemented.

The molecule prediction system 100 is configured process molecule data 102 to generate an output prediction 104 for one or more molecules represented by the molecule data 102. For example, the molecule prediction system 100 can process the molecule data 102 to generate the output prediction 104 for a molecule complex that includes the molecules represented by the molecule data 102.

As an example, the molecule data 102 can include data characterizing a protein and a ligand and the molecule prediction system 100 can generate the output prediction 104 for a protein-ligand complex that includes the protein and the ligand.

As another example, the molecule data 102 can include data characterizing a first protein and a second protein and the molecule prediction system 100 can generate the output prediction 104 for a protein-protein complex that includes the first protein and the second protein.

As another example, the molecule data 102 can include protein data for a protein that includes any appropriate data characterizing the protein, e.g., data defining one or more amino acid sequences of the protein, or data defining an MSA (Multiple Sequence Alignment) for the protein (e.g. defining correspondence between amino acids of the protein and those of other, homologous proteins), or data characterizing a respective structure of each of one or more “template” proteins, or a combination thereof. A template protein can refer to a protein that is “similar” to the protein, e.g., such that the value of a similarity measure between the template protein and the protein satisfies (e.g., exceeds) a threshold (e.g., 0.8, or 0.9, or 0.99, or any other appropriate threshold). Similarity between a first protein and a second protein can be measured using any appropriate similarity measure, e.g., a sequence identity or percent identity similarity measure between the respective amino acid sequence(s) of the first protein and the second protein. The structure of a template protein can be represented in any appropriate manner, e.g., by a contact map, or by data defining a respective 3D spatial position of each atom in the template protein.

As another example, the molecule data 102 can include ligand data for a ligand that includes any appropriate data characterizing the ligand. A ligand can refer to a molecule or compound that binds to a target molecule, e.g., a protein. Ligands can include, e.g., small organic molecules, complex organic molecules, proteins, biomolecules, and so forth. For instance, the ligand data can include a textual representation of one or more of: a chemical structure of the ligand (e.g., the arrangement of atoms and bonds in the ligand), the atom types in the ligand and their connectivity, the chirality of the bonds in the ligand, or any functional groups (e.g., hydroxyl groups, amino groups, carboxyl groups, and so forth) included in the ligand. The textual representation of the ligand can include, e.g., a simplified molecular-input line-entry system (SMILES) string characterizing the ligand. As another example, the ligand data can include a representation of the ligand by way of graph data representing a graph, e.g., where the nodes in the graph represent atoms in the ligand and the edges in the graph represent bonds between atoms in the ligand. Optionally, the ligand data can exclude any data that directly defines the 3D structure of each ligand, e.g., the 3D spatial locations of the atoms in a 3D conformation of the ligand.

In some cases, the molecule data 102 characterizes a single molecule (e.g., a small molecule, or a protein molecule, or a deoxyribonucleic acid (DNA) molecule, or a ribonucleic acid (RNA) molecule, and so forth). In other cases, the molecule data 102 can characterize multiple molecules, e.g., 2 molecules, or 3 molecules, or 5 molecules, or 10 molecules, or any other appropriate number of molecules.

In some implementations, the molecule data 102 can include molecule design criteria for a ligand to be designed by the molecule prediction system 100 (e.g., a designed ligand predicted to bind with a protein specified by the molecule data 102). The molecule design criteria can (but need not) specify, e.g., target properties for the designed ligand, scaffolding data for the designed ligand, and so on.

As part of generating the output prediction 104 for the one or more molecules represented by the molecule data 102, the molecule prediction system 100 can generate a joint molecule embedding 106 that jointly represents the one or more molecules represented by the molecule data 102. In particular, the molecule prediction system 100 can process the molecule data 102 using an embedding neural network 108 to generate the joint molecule embedding 106 for the one or more molecules represented by the molecule data 102. In this specification, although for convenience reference is made to a joint molecule embedding, in some instances the joint molecule embedding referred to is an embedding generated by processing molecule data characterizing just one molecule. In general a molecule embedding can be an ordered collection of numerical values, e.g., a vector, matrix, or other tensor of numerical values.

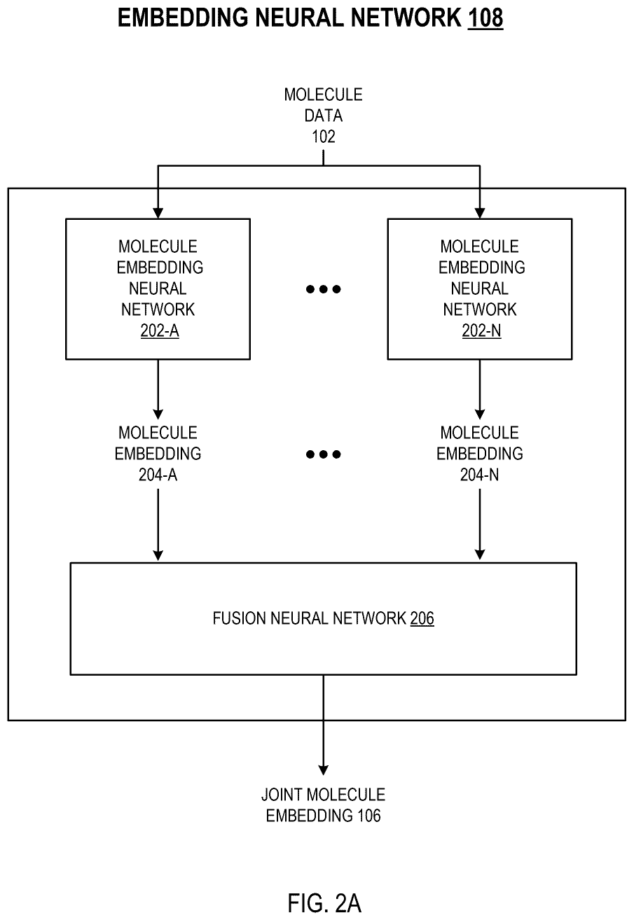

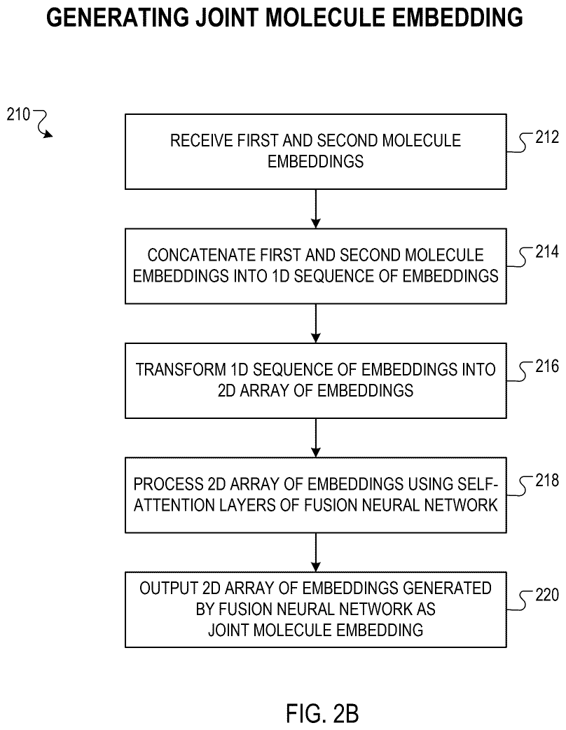

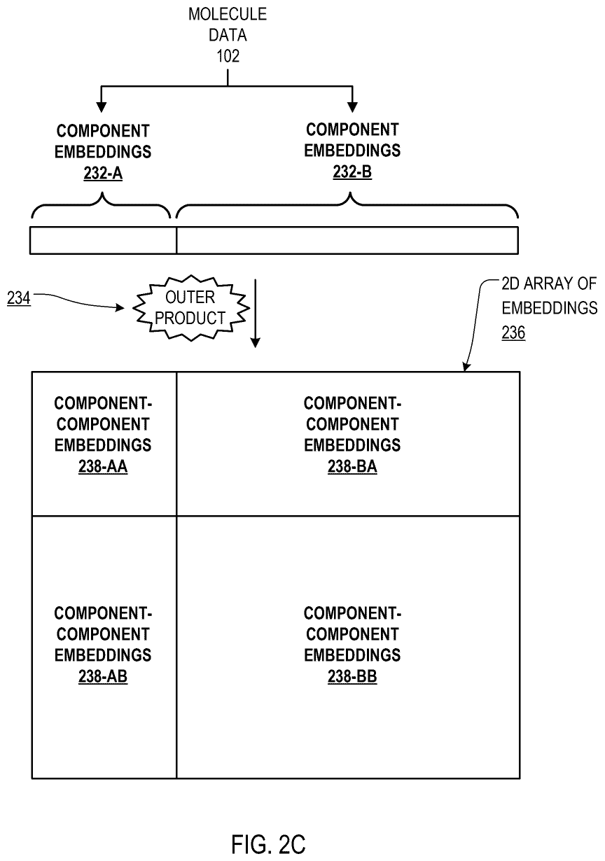

The embedding neural network 108 can have any appropriate neural network architecture that enables the embedding neural network 108 to perform its described functions. In particular, the embedding neural network 108 can include any appropriate types of neural network layers (e.g., fully connected layers, convolutional layers, attention layers, etc.) in any appropriate number (e.g., 5 layers, 10 layers, or 20 layers) and connected in any appropriate configuration (e.g., as a directed graph of layers). An example architecture of the embedding neural network is described in more detail with reference to FIG. 2A.

The molecule prediction system 100 can include a plurality of prediction neural networks 110-A through 110-N. Each of the plurality of prediction neural networks 110-A through 110-N can be configured to process the joint molecule embedding 106 generated by the embedding neural network 108 to generate respective predictions 112-A through 112-N for the molecule(s) represented by the joint molecule embedding 106. The embedding neural network 108 can be jointly trained with the plurality of prediction neural networks 110-A through 110-N, e.g., by a training engine 114 of the molecule prediction system 100.

The molecule prediction system 100 can jointly train the embedding neural network 108 and the plurality of prediction neural networks on a set of training data using an appropriate machine learning training technique. For each of the plurality of prediction neural networks, the training data can include a set of training examples for the prediction neural network. Each training example for a prediction neural network can include (i) a training input that characterizes one or more molecules for the training example, and (ii) a target output for the prediction neural network (e.g., a target predicted property for a molecule or molecule complex of the training example, a target predicted 3D structure for a molecule or molecule complex of the training example, target atoms and bonds for a designed molecule for the training example, etc.).

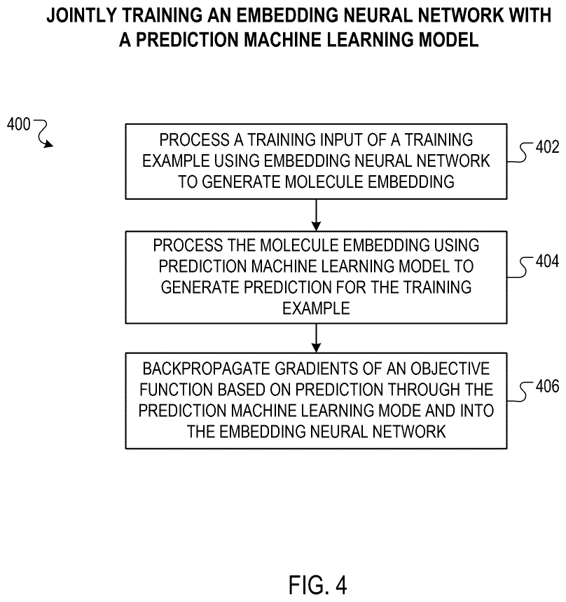

For example, the machine learning training technique can include processing the training input of the training example using the embedding neural network to generate a joint molecule embedding for the training example. The molecule prediction system 100 can then process the joint molecule embedding for the training example using the prediction neural network associated with the training example to generate an output prediction for the training example. The molecule prediction system 100 can backpropagate gradients of an objective function for the prediction neural network through the prediction neural network and into the embedding neural network. The objective function for the prediction neural network can measure a discrepancy between the output prediction generated by the prediction neural network and the target output specified by the training example. An example process for jointly training the embedding neural network and a prediction neural network is described in more detail with reference to FIG. 4.

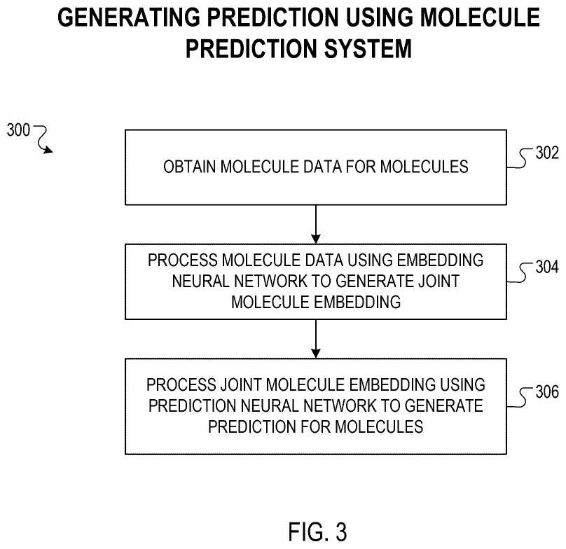

The molecule prediction system 100 can include a prediction machine learning model 116 configured to process the joint molecule embedding 106 to generate the output prediction 104. The prediction machine learning model 116 can have any of a variety of machine learning architectures. As an example, the prediction machine learning model 116 can be one of the plurality of prediction neural networks 110-A through 110-N jointly trained with the embedding neural network 108. As another example, the prediction machine learning model 116 can be a prediction neural network that has been trained separately from the embedding neural network 108. As another example, the prediction machine learning model 116 can be a non-differentiable machine learning model (e.g., a random forest or a support vector machine (SVM)) configured to process the joint molecule embedding 106 to perform a prediction task. An example process for generating an output prediction using the molecule prediction system 100 is described in more detail below with reference to FIG. 3.

The prediction machine learning model 116 and the plurality of prediction neural networks 110-A through 110-N can perform any of a variety of prediction tasks for the molecules represented by the molecule data 102. A number of example prediction tasks are described next.

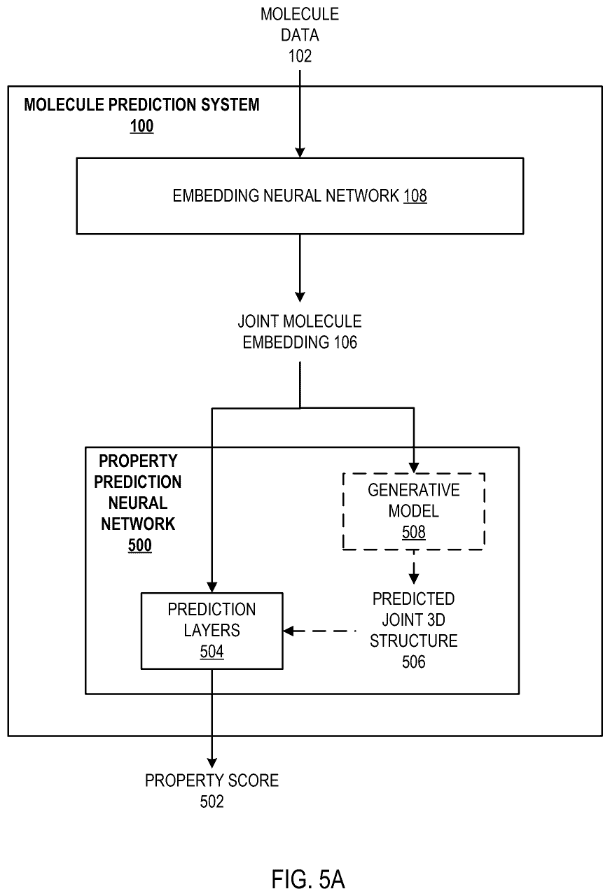

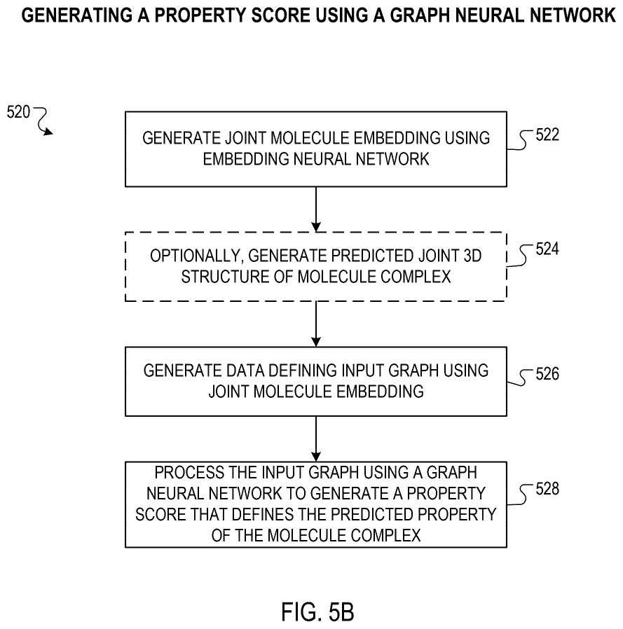

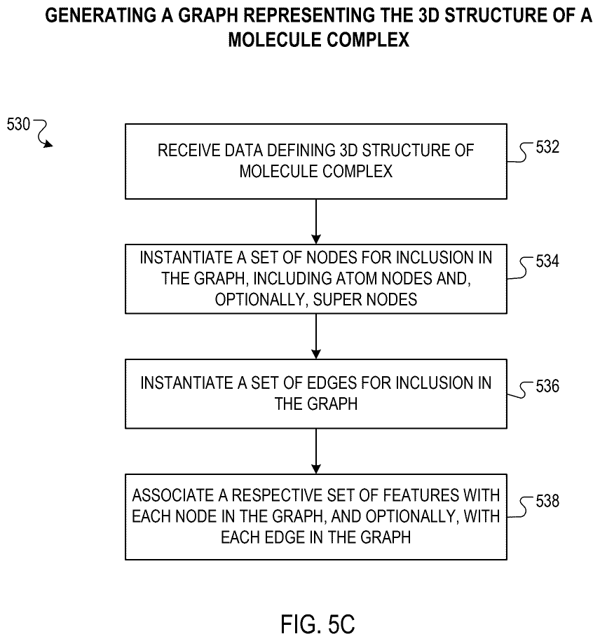

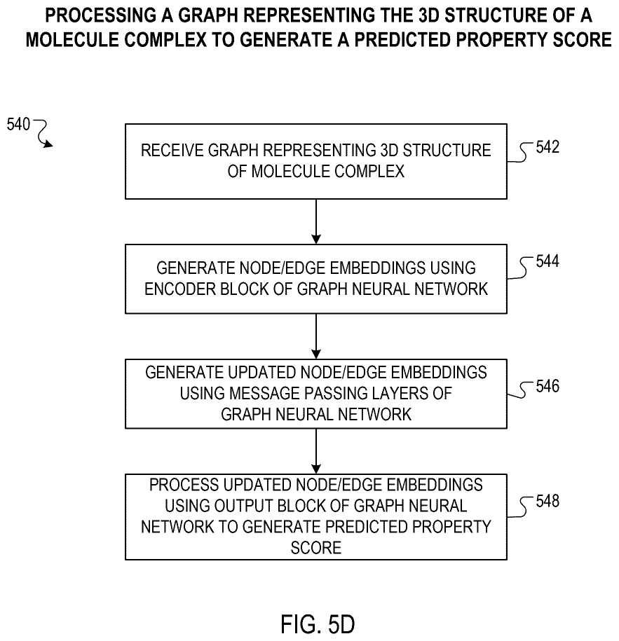

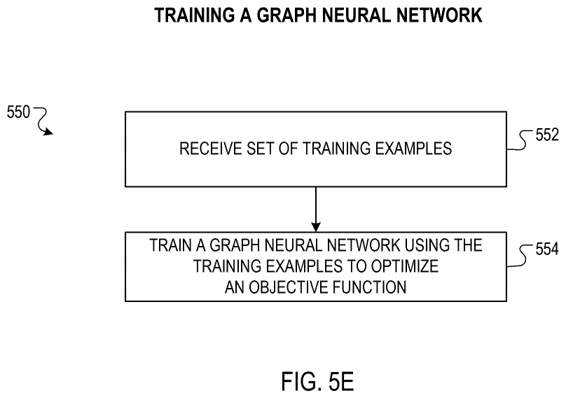

As an example, the prediction neural networks 110-A through 110-N can include a property prediction neural network. The property prediction neural network can process the joint molecule embedding 106 and predict a property for one or more molecules represented by the molecule data 102. As an example, the property prediction neural network can process the joint molecule embedding 106 and predict a property for a particular molecule represented by the molecule data 102 (e.g., generate a predicted absorption, distribution, metabolism, excretion, toxicity value, etc., for the particular molecule). As another example, the property prediction neural network can process the joint molecule embedding 106 and generate a value of a predicted property (e.g., a binding affinity, a binding energy, etc.) for a complex including the molecules represented by the molecule data 102. An example property prediction neural network is described in more detail below with reference to FIG. 5A-FIG. 5D.

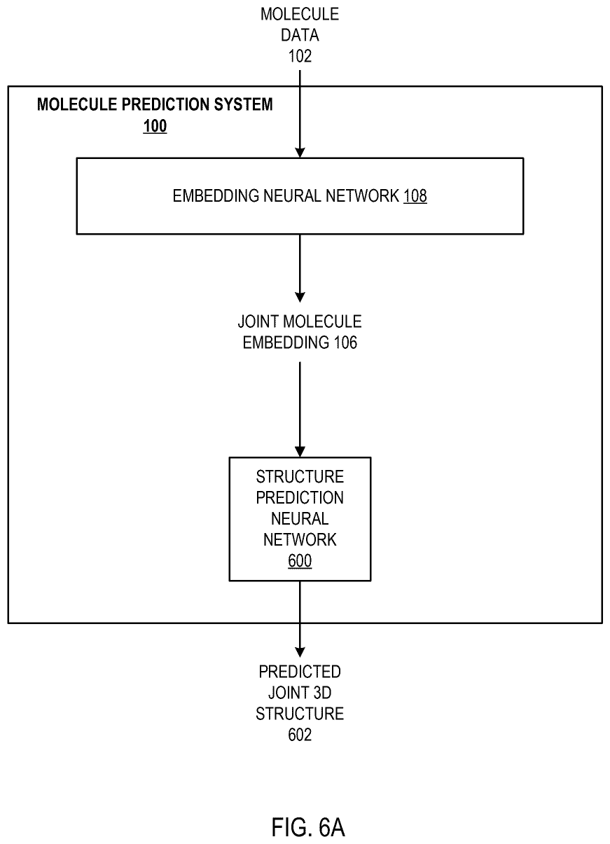

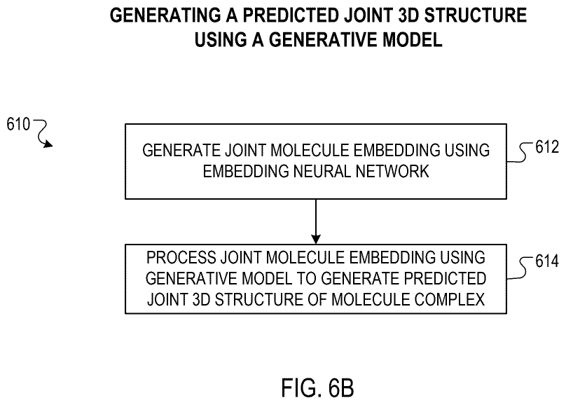

As another example, the prediction neural networks 110-A through 110-N can include a structure prediction neural network. The structure prediction neural network can process the joint molecule embedding 106 and generate a predicted 3D structure for one or more molecules represented by the molecule data 102. For example, the structure prediction neural network can process the joint molecule embedding 106 and generate a predicted 3D structure for a particular molecule represented by the molecule data 102. As another example, the structure prediction neural network can process the joint molecule embedding 106 and generate a predicted 3D structure (e.g., a predicted joint 3D structure) for a molecule complex including the molecules represented by the molecule data 102. An example structure prediction neural network is described in more detail below with reference to FIG. 6A-FIG. 6B. Additional examples of structure prediction neural networks are described in: Abramson, J. et al., “Accurate structure prediction of biomolecular interactions with AlphaFold 3.” Nature, 630, pages 493-500 (2024). DOI: 10.1038/s41586-024-07487-w; Jumper, J. et al., “Highly accurate protein structure prediction with AlphaFold.” Nature, 596, pages 583-589 (2021). DOI: 10.1038/s41586-021-03819-2; or Back, M. et al., “Accurate prediction of protein structures and interactions using a 3-track network,” Science (2021) DOI: 10.1126/science.abj8754.

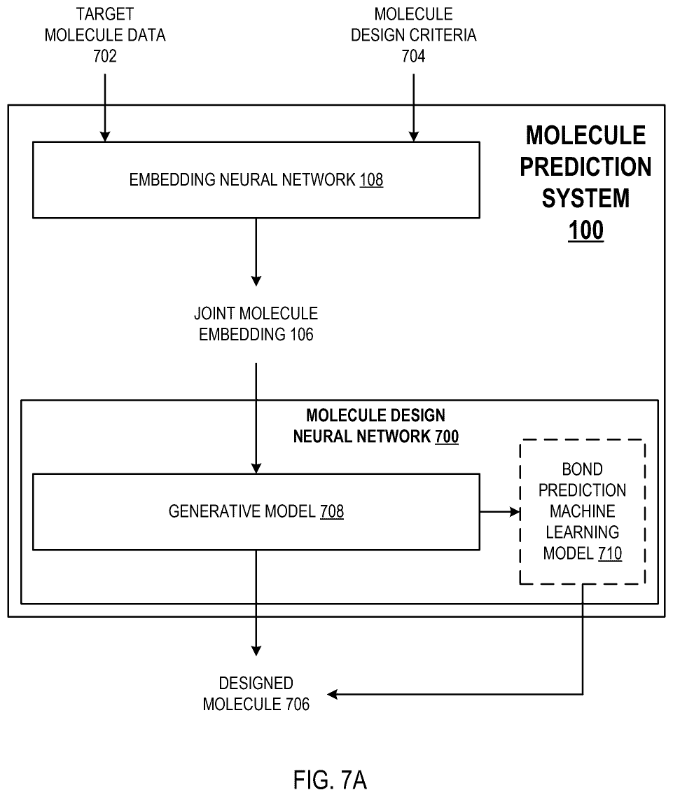

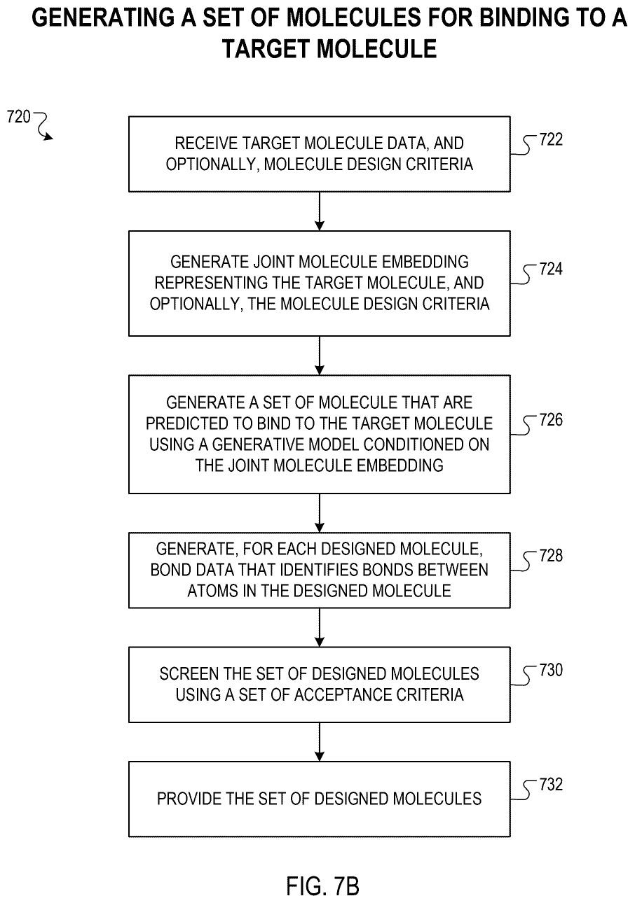

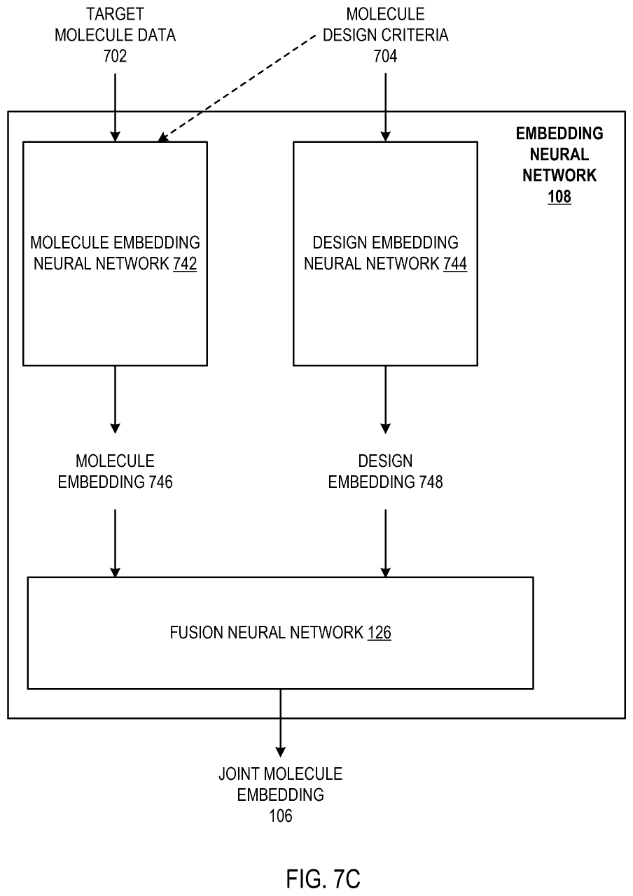

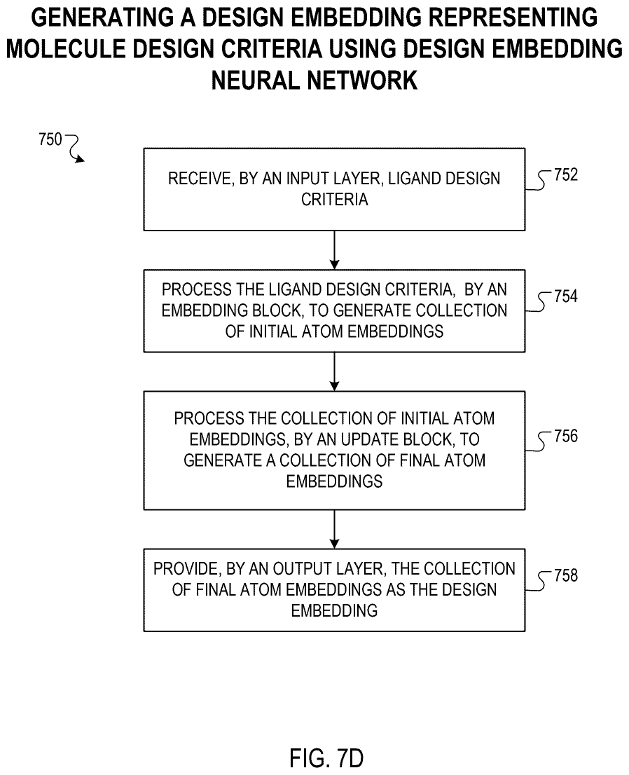

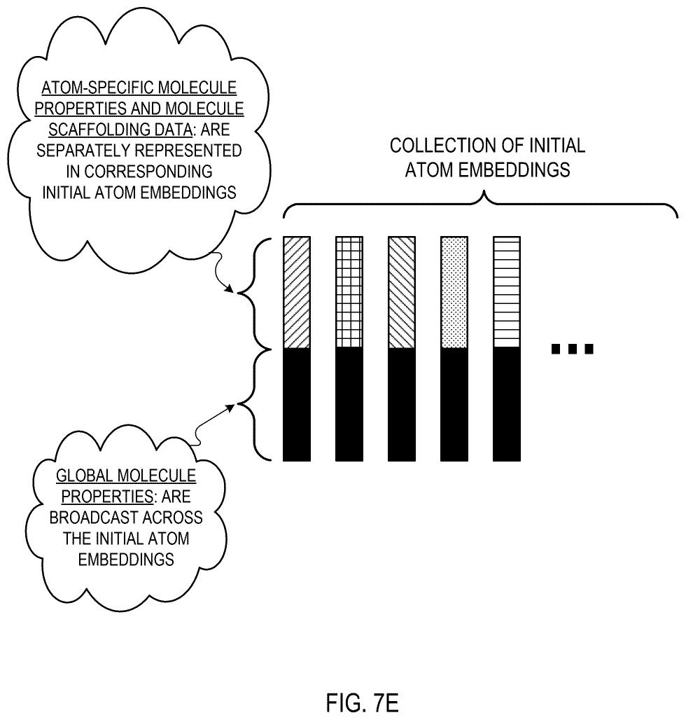

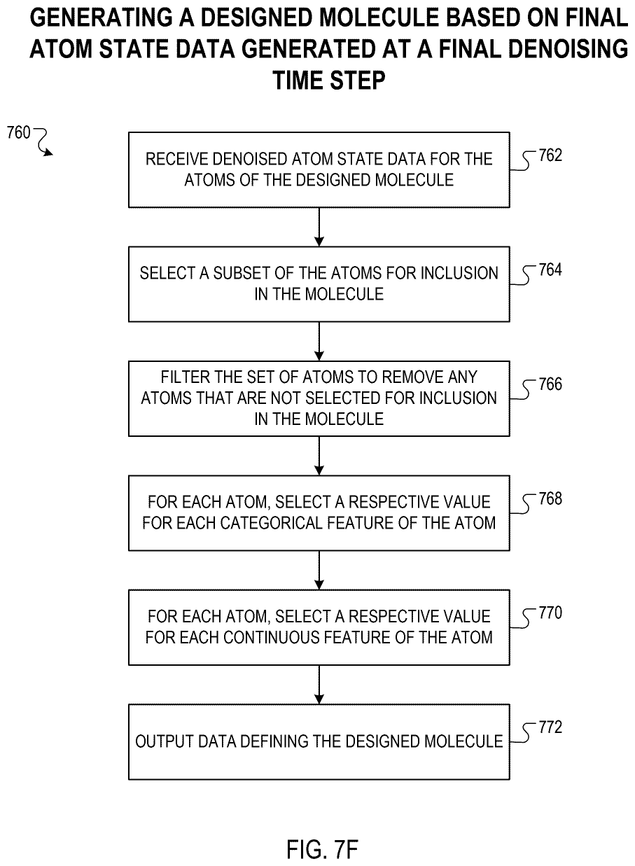

As another example, the prediction neural networks 110-A through 110-N can include a molecule design neural network (e.g., a ligand design neural network). When the molecule data 102 characterizes a protein, the molecule design neural network can process the joint molecule embedding 106 and generate data characterizing a ligand designed to bind with the protein represented by the molecule data 102. An example molecule design neural network is described in more detail below with reference to FIG. 7A-FIG. 7F. Additional examples of molecule design neural networks are described in Watson et al., “Broadly Applicable and Accurate Protein Design by Integrating Structure Prediction Neural Networks and Diffusion Generative Models” bioRxiv 2022.12.09.519842.

As another example, the prediction neural networks 110-A through 110-N can include a distogram prediction neural network. The distogram prediction network can process the joint molecule embedding 106 and generate data characterizing a predicted distogram for a plurality of molecular components (e.g., atoms, groups of atoms, amino acid residues, nucleic acid residues, etc.) of the one or more molecules represented by the molecule data 102. The predicted distogram can characterize, for each pair of the molecular components of the one or more molecules represented by the molecule data 102, a histogram of predicted probabilities of distances between the pair of molecular components.

As another example, the prediction neural networks 110-A through 110-N can include a contact probability prediction neural network. The contact probability prediction network can process the joint molecule embedding 106 and generate data characterizing a predicted contact probability for a plurality of molecular components (e.g., atoms, groups of atoms, amino acid residues, nucleic acid residues, etc.) of the one or more molecules represented by the molecule data 102. For each pair of the molecular components of the one or more molecules represented by the molecule data 102, the predicted contact probability for the pair of molecular components can characterize a predicted probability for the pair of molecular components to contact one another (e.g., be within a pre-defined threshold distance of one another).

The plurality of prediction neural networks 110-A through 110-N can include multiple prediction neural networks having a same network architecture that have each been trained using a different set of training data. For instance, the prediction neural networks 110-A through 110-N can include multiple prediction neural networks that are each specialized to generate predictions for to a particular class of molecule (e.g., a particular class of protein, such as G Protein Coupled Receptors) as a result of being trained using training data for that class of molecule.

The molecule prediction system 100 can receive the molecule data 102 from any appropriate source, e.g., from a user or from another system, by way of an appropriate interface, e.g., an application programming interface (API), a user interface (e.g., a graphical user interface), and so on. After generating the output prediction 104, the molecule prediction system 100 can, e.g., store data defining the output prediction 104 in a memory, transmit data defining the output prediction 104 over a data communication network, provide data defining the predictions, output prediction 104 directly to a system that performs downstream processing based on the output prediction 104, and so on.

The output prediction 104 generated by the molecule prediction system 100 can be used in any of a variety of possible downstream applications. A few examples of downstream applications that process predictions generated by the molecule prediction system 100 are described next.

In some cases, the output prediction 104 generated by the molecule prediction system 100 can be used for drug discovery. Drug discovery can involve identifying specific molecules within the body that are involved in a disease process. These molecules are often proteins, such as enzymes, receptors, or signaling proteins, that play a key role in the disease's development or progression. A ligand, often a small molecule, peptide, or antibody, can be selected to bind specifically to an identified target protein. When a drug that includes the ligand is administered to a patient, the ligand can bind to the target protein with high affinity and in doing so contribute to achieving a therapeutic effect in the patient. For instance, if the target protein is an enzyme involved in a disease process, the ligand can inhibit its activity, thus disrupting the disease pathway. More generally, the interaction between the ligand and the target protein can activate, inhibit, or alter the function of the target protein to achieve a therapeutic effect.

Therefore, identifying ligands with (relatively) high (or low) binding affinity for a protein can be an important step in the process of drug discovery. (Identifying ligands with low binding affinities for a protein can be desirable, e.g., when the protein is an off-target protein and the binding of the ligand to the protein may cause undesirable side effects). However, determining binding affinities of ligands for proteins, e.g., through computational simulations or physical experiments, can be expensive and time consuming.

To address these issues, the output prediction 104 generated by the molecule prediction system 100 can be used to determine a ranking of candidate ligands in a collection of candidate ligands based on respective predicted property scores (e.g., binding affinities for a protein) for the candidate ligands. More specifically, for each candidate ligand, the molecule prediction system 100 can generate a respective predicted property score of the candidate ligand for the protein. The collection of candidate ligands can then be ranked based on the respective predicted property score of each candidate ligand. Such a predicted property score can be a relative predicted property score, e.g. a relative predicted binding affinity between a protein and a ligand, e.g. amongst a set of candidate ligands for the protein or amongst a set of candidate proteins for a particular ligand.

As an example, the molecule prediction system 100 can include a property prediction neural network configured to generate the predicted property scores for the candidate ligand and can rank the collection of candidate ligands using the predicted property scores generated by the property prediction neural network.

As another example, the molecule prediction system 100 can include a structure prediction neural network configured to generate predicted joint 3D structures for protein-ligand complexes including the candidate ligands. The predicted joint 3D structure (or features derived from the predicted joint 3D structure) for each of the candidate ligands can be processed by a scoring function to generate a predicted property score (e.g., a predicted binding affinity) for the candidate ligand and the protein. The collection of candidate ligands then be ranked based on the respective predicted property scores generated based on the predicted joint 3D structures.

In some cases, the scoring function can generate the predicted property score based on factors such as an electrostatic interaction factor, a van der Waals forces factor, a hydrophobic interaction factor, a lipophilic interaction factor, and so forth, that are each derived from the predicted joint 3D structure. In some cases, the scoring function can be a machine learning model that is configured to process the predicted joint 3D structure (or features derived from the predicted joint 3D structure) in accordance with values of a set of machine learning model parameters to generate the predicted property score.

The ranking of the candidate ligands in the collection of candidate ligands based on their respective predicted property scores can be used, e.g., to select a proper subset of the collection of candidate ligands for experimental validation and testing. For instance, one or more candidate ligands having the highest or lowest predicted property scores (i.e., according to the ranking) can be selected for experimental validation and testing, e.g., for use in a drug that achieves a therapeutic effect in patients. In particular, each selected candidate ligand can be physically synthesized and then tested for a variety of physio-chemical properties, e.g., absorption properties, distribution properties, metabolism properties, excretion properties, binding affinities with target proteins, and so on. One or more of the candidate ligands from the collection of ligands can be selected for inclusion in a drug, e.g., based at least in part on results of the testing. A drug that includes one or more of the candidate ligands can be synthesized using any appropriate drug synthesis technique. In general references herein to synthesis can refer to manual and/or automatic synthesis, e.g. using robotic techniques.

The collection of candidate ligands can include any appropriate number of ligands, e.g., 10 ligands, or 1000 ligands, or 100,000 ligands. In some cases, only a fraction of the candidate ligands in the set of candidate ligands are selected for physical synthesis, e.g., based on the ranking of the candidate ligands by their predicted property scores. For instance, less than 50%, or less than 10%, or less than 1%, or less than 0.1% of the candidate ligands in the collection of candidate ligands may be selected for physical synthesis.

The candidate ligand(s) may be derived from a database of candidate ligands, and/or may be derived by modifying ligands in a database of candidate ligands, e.g., by modifying a structure or amino acid sequence of a candidate ligand, and/or may be derived by stepwise or iterative assembly/optimization of a candidate ligand. The candidate ligand(s) may alternately or additionally include one or more candidate ligands generated using a generative model conditioned on (the structure of) the target protein molecule or part of the target protein molecule, e.g. a structure of a binding site or other part of the target protein molecule.

In some implementations the candidate ligand(s) may include small molecule complex ligands, e.g., organic compounds with a molecular weight of <900 daltons. In some other implementations the candidate ligand(s) may include polypeptide ligands, i.e., defined by an amino acid sequence.

In some implementations a candidate (e.g. polypeptide or polynucleotide) ligand may include: an isolated antibody or aptamer, a fragment of an isolated antibody or aptamer, a single variable domain antibody, a bi- or multi-specific antibody, a multivalent antibody, a dual variable domain antibody, an immuno-conjugate, a fibronectin molecule, an adnectin, an DARPin, an avimer, an affibody, an anticalin, an affilin, a protein epitope mimetic or combinations thereof. A candidate (polypeptide) ligand may include an antibody with a mutated or chemically modified amino acid Fc region, e.g., which prevents or decreases ADCC (antibody-dependent cellular cytotoxicity) activity and/or increases half-life when compared with a wild type Fc region. Candidate (polypeptide or polynucleotide) ligands may include antibodies with different CDRs (Complementarity-Determining Regions).

In some implementations, one or more selected ligands can by synthesized, i.e., by making, the small molecule, polynucleotide or polypeptide ligand. The one or more ligands may be synthesized by any conventional chemical techniques and/or may already be available, e.g., may be from a compound library or may have been synthesized using combinatorial chemistry.

One or more selected ligands can be tested for biological activity in vitro and/or in vivo. For example each selected ligand may be tested for ADME (absorption, distribution, metabolism, excretion) and/or toxicological properties (ADMET), to screen out unsuitable ligands. The testing may include, e.g., bringing the candidate small molecule, polypeptide or polynucleotide ligand into contact with the target protein molecule and measuring a change in expression or activity of the target molecule.

In some cases, the output prediction 104 generated by the molecule prediction system 100 can be used for drug repurposing. In more detail, an existing drug may include a particular ligand, e.g., that is known to achieve a therapeutic effect in patients by binding to a target protein involved with a particular disease process. Drug repurposing can involve identifying new protein binding targets for the ligand, e.g., that are potentially involved in different disease processes. If the ligand has a high binding affinity (or potency, or inhibitory effect, etc.) for a new target protein that is involved in a disease process, then the ligand can be selected for experimental validation and potential inclusion in a drug for treating the disease. Drug repurposing can leverage the safety and efficacy data already available for a drug that includes the ligand, potentially accelerating the development process and reducing research costs. Drug repurposing can identify novel treatment options and address unmet medical needs by repurposing known ligands to treat different diseases or conditions.

To identify new target proteins for a ligand, the output prediction 104 generated by the molecule prediction system 100 can be used to determine a ranking of candidate proteins in a collection of candidate proteins based on a respective predicted property score (e.g., a binding affinity) of a particular ligand relative to each of the candidate proteins. More specifically, for each candidate protein, the molecule prediction system 100 can generate a respective predicted property score for the ligand and the candidate protein. The collection of candidate proteins can then be ranked based on the respective predicted property score of the ligand relative to each of the candidate proteins.

As an example, the molecule prediction system 100 can include a property prediction neural network configured to generate the predicted property scores for the candidate proteins and can rank the collection of candidate proteins using the predicted property scores generated by the property prediction neural network.

As another example, the molecule prediction system 100 can include a structure prediction neural network configured to generate predicted joint 3D structures for protein-ligand complexes including the candidate proteins. For each candidate protein, a scoring function can process the predicted joint 3D structure of the ligand and the candidate protein to generate a predicted property score of the ligand for the candidate protein. The collection of candidate proteins can then be ranked based on the respective predicted property score of the ligand for each of the candidate proteins generated based on the predicted joint 3D structures.

The ranking of the candidate proteins in the collection of candidate proteins based on the predicted property scores of the ligand relative to the candidate proteins can be used, e.g., to select a proper subset of the collection of candidate proteins for experimental validation and testing. For instance, one or more candidate proteins for which the ligand has the highest predicted property score (i.e., according to the ranking) can be selected for experimental validation and testing, e.g., for use in a drug that achieves a therapeutic effect in patients. In particular, each selected candidate protein can be physically synthesized and the properties of the ligand relative to the candidate protein can then be experimentally tested and validated. One or more of the candidate proteins from the collection of candidate proteins can be selected as binding targets for the ligand, e.g., based at least in part on results of the testing.

The collection of candidate proteins can include any appropriate number of proteins, e.g., 10 proteins, or 1000 proteins, or 100,000 proteins. In some cases, only a fraction of the candidate proteins in the set of candidate proteins are selected for physical synthesis, e.g., based on the ranking of the candidate proteins by the predicted property score of the ligand relative to the candidate proteins. For instance, less than 50%, or less than 10%, or less than 1%, or less than 0.1% of the candidate proteins in the collection of candidate proteins may be selected for physical synthesis.

When output prediction 104 includes one or more generated ligands designed to bind with a target protein, the generated ligands can be used for drug design. For example, the generated ligands can be predicted to bind to a target protein that is identified as being involved in a disease process, e.g., associated with cancer, Alzheimer's disease, heart disease, infectious diseases (e.g., bacterial diseases, viral diseases, parasitic diseases, fungal diseases, prion diseases, etc.), and so forth. By binding to the protein, a ligand can modulate (e.g., inhibit or activate) the activity of the protein thereby disrupting the disease process and contributing to treating the disease.

As another example, generated ligands can be used for pest or pathogen control in agriculture. For example, the generated ligands can be predicted to bind to proteins in agricultural pests or pathogens (e.g., insects, fungi, or bacteria). Such ligands can be used as part of targeted pesticides that bind to proteins that are essential to the survival of pests or pathogens and that are not found in non-target species.

As another example, the generated ligands can be used for modifying plant specifies cultivated for agricultural purposes. For example, the generated ligands can be predicted to bind to proteins identified as being involved in plant growth, or stress resistance, or both. (Stress resistance in a plant species can characterize an ability of the plant species to survive and adapt to adverse conditions such as drought, high soil salinity, extreme temperatures, and so forth). Such ligands can be used for modulating the behavior of target proteins in the plant species to increase crop yields and stress resistance.

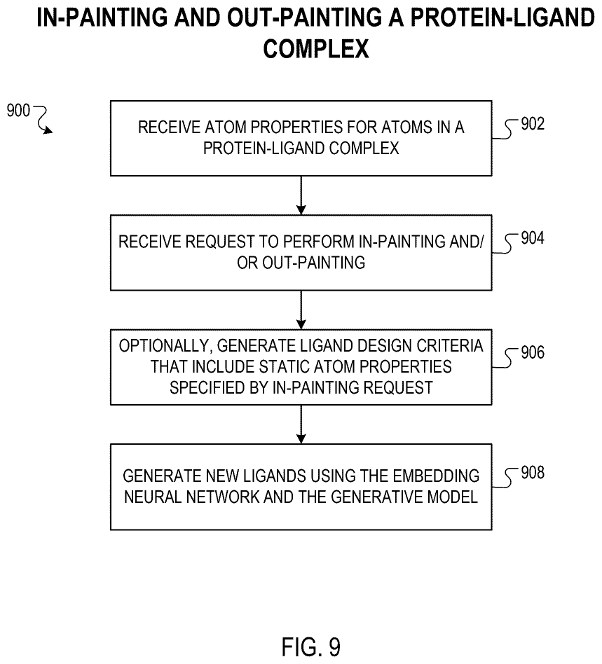

In some implementations, the molecule prediction system 100 can receive requests to perform “in-painting” or “out-painting” of a protein-ligand complex. A request to in-paint the complex identifies, for each atom property of each atom in the complex, whether the atom property is a “static” property or a “variable” property. In-painting the complex refers to generating new ligands that have the static atom properties of the input ligand and that bind to proteins that have the static atom properties of the input protein. Out-painting the complex refers to generating new ligands that expand on the original ligand, e.g., by inclusion of one or more new atoms. An example process for in-painting and out-painting a complex is described with reference to FIG. 9.

FIG. 2A shows an example embedding neural network 108, e.g., that is included in the molecule prediction system described with reference to FIG. 1. The embedding neural network 108 is configured to process molecule data 102 characterizing one or more molecules to generate a joint molecule embedding 106 of the molecules represented by the molecule data 102.

As an example, the molecule data 102 can represent a protein and a ligand and the molecule prediction system 100 can generate a joint molecule embedding 106 for a protein-ligand complex that includes the protein and the ligand. As another example, the molecule data 102 can represent a first protein and a second protein and the molecule prediction system 100 can generate a joint molecule embedding 106 for a protein-protein complex that includes the first protein and the second protein.

In general, the molecule data 102 can represent one or more of a variety of molecules and the molecule prediction system 100 can generate the joint molecule embedding 106 representing the molecules represented by the molecule data 102. For example, the molecule data 102 can represent one or more, e.g., ligands, proteins, nucleic acids, and so on in any combination and the molecule prediction system 100 can generate the joint molecule embedding 106 representing the combination of molecules represented by the molecule data 102.

The embedding neural network 108 can include one or more molecule embedding neural networks 202-A through 202-N and, in some implementations, a fusion neural network 206, which are each described in more detail next (and throughout this specification).

The molecule embedding neural networks 202-A through 202-N are configured to process the molecule data 102 respectively to generate respective molecule embeddings 204-A through 204-N of the respective molecules represented by the molecule data 102.

The molecule embedding neural networks 202-A through 202-N can have any appropriate neural network architecture that enables molecule embedding neural networks 202-A through 202-N to perform their described functions. In particular, the molecule embedding neural networks 202-A through 202-N can include any appropriate types of neural network layers (e.g., fully connected layers, convolutional layers, attention layers, etc.) in any appropriate number (e.g., 5 layers, 10 layers, or 20 layers) and connected in any appropriate configuration (e.g., as a directed graph of layers).

For example, the molecule embedding neural networks 202-A through 202-N can include protein embedding neural networks configured process molecule data characterizing a protein to generate a protein embedding for the protein. A protein embedding for a protein can include, e.g., a respective amino acid embedding of each amino acid in each amino acid sequence of the protein. Particular examples of possible architectures of a protein embedding neural network include the “Pairformer” neural network described in Abramson et al., “Accurate structure prediction of biomolecular interactions with AlphaFold 3,” Nature, Vol 630, 8 May 2024, and the “Evoformer” neural network described in Jumper et al., “Highly accurate protein structure prediction with AlphaFold,” Nature, Vol 596, 26 Aug. 2021. The Pairformer and the Evoformer can process a network input derived from: (i) the amino acid sequence of a protein, (ii) an MSA for the protein, and (iii) the 3D structures of one or more template amino acid sequences, to generate an output that includes a “single representation” that defines a respective embedding of each position in each amino acid sequence of the protein. (A template amino acid sequence an MSA sequence for an amino acid chain in the protein where the folded structure of the template sequence is known, e.g., from physical experiments). In some implementations, the protein embedding for the protein can include a respective atom embedding for each of one or more atoms included within the protein.

As another example, the molecule embedding neural networks 202-A through 202-N can include a ligand embedding neural network configured to process molecule data characterizing a ligand to generate a ligand embedding of the ligand. A ligand embedding of a ligand can include a respective atom embedding representing each atom in the ligand. In a particular example, the ligand embedding neural network can be configured to receive a collection of initial atom embeddings that includes a respective initial atom embedding for each atom in the ligand and that is derived from data characterizing the ligand, e.g., a SMILES string representing the ligand. The initial atom embedding of each atom can include data characterizing the type of the atom, the other atoms to which the atom is bonded, whether the atom is included in any functional groups, and so forth. In some implementations, the ligand embedding neural network can be configured to process additional conditioning data characterizing the ligand, e.g., data characterizing 3-dimensional conformers of the ligand, data characterizing a chirality of the ligand, data characterizing a hybridization of atoms of the ligand, and so on. The ligand embedding neural network can process the collection of initial atom embeddings by a sequence of one or more attention neural network layers, that are each configured to update the collection of current atom embeddings by a self-attention operation, to generate the embedding of the ligand, e.g., as the collection of atom embeddings output by a final attention layer in the sequence of attention layers.

As another example, the molecule embedding neural networks 202-A through 202-N can include nucleic acid embedding neural networks configured process molecule data characterizing a nucleic acid to generate a nucleic acid embedding for the protein. A nucleic acid embedding for a nucleic acid can include, e.g., a respective nucleotide embedding of each nucleotide in a nucleotide sequence of the nucleic acid. Particular examples of possible architectures of a protein embedding neural network include adaptations of the “Pairformer” neural network described in Abramson et al., “Accurate structure prediction of biomolecular interactions with AlphaFold 3,” Nature, Vol 630, 8 May 2024, and of the “Evoformer” neural network described in Jumper et al., “Highly accurate protein structure prediction with AlphaFold,” Nature, Vol 596, 26 Aug. 2021. In some implementations, the nucleic acid embedding for the nucleic acid can include a respective atom embedding for each of one or more atoms included within the nucleic acid.

The fusion neural network 206 is configured to process the molecule embeddings 204-A through 204-N to generate the joint molecule embedding 106 that jointly represents the molecules represented by the molecule data 102. The fusion neural network 206 can have any appropriate neural network architecture that enables the fusion neural network 206 to perform its described functions. In particular, the fusion neural network 206 can include any appropriate types of neural network layers (e.g., fully connected layers, convolutional layers, attention layers, etc.) in any appropriate number (e.g., 5 layers, 10 layers, or 20 layers) and connected in any appropriate configuration (e.g., as a directed graph of layers). An example of processing a molecule embeddings to generate a joint molecule embedding 106 is described in more detail with reference to FIG. 2B.

In some implementations, the fusion neural network 206 can include multiple sub-networks (e.g., processing layers, blocks, etc.) that are distributed across multiple computing devices. In particular, the fusion neural network 206 can be configured to generate the joint molecule embedding 106 by performing data sharding across the multiple computing devices, as described by Lepikhin et al. in “GShard: Scaling Giant Models with Conditional Computation and Automatic Sharding”. For example, to reduce or parallelize the computational cost of generating the joint molecule embedding 106, the fusion neural network 206 can process different sub-sets (e.g., micro-batches) of embeddings from the molecule embeddings 202-A through 202-N as different shards that are distributed across the computational devices for the fusion neural network 206.

FIG. 2B is a flow diagram of an example process 210 for processing a molecule embedding of a first molecule and a molecule embedding of a second molecule using a fusion neural network to generate a joint molecule embedding that jointly represents the first molecule and the second molecule. For convenience, the process 210 will be described as being performed by a system of one or more computers located in one or more locations. For example, a molecule prediction system, e.g., the molecule prediction system 100 of FIG. 1, appropriately programmed in accordance with this specification, can perform the process 210.

The system receives a first molecule embedding of a first molecule and a second molecule embedding of a second molecule (212). The first and second molecule embeddings can be generated by a molecule embedding neural network. For example, the first and second molecules can include a protein and the first and second molecule embeddings can include a protein embedding generated by a protein embedding neural network. As another example, the first and second molecules can include a ligand and the first and second molecule embeddings can include a ligand embedding generated by a ligand embedding neural network.

The first and second molecule embeddings can each include a plurality of component embeddings that each represent a respective molecule component (e.g., an atom, an amino acid, a nucleic acid, etc.) of the first and second molecules. For example, a protein embedding of a protein can include a respective amino acid embedding for each position in each amino acid sequence of the protein. As another example, a ligand embedding of a ligand can include a respective atom embedding for each atom in the ligand. As another example, a nucleic acid embedding of a nucleic acid can include a respective nucleotide embedding for each nucleotide in the nucleic acid.

The system concatenates the first molecule embedding and the second molecule embedding to generate a one-dimensional (1D) sequence of embeddings (214). The 1D sequence of embeddings includes the component embeddings included within the first and second molecule embeddings. For example, when the first and second molecule embeddings include a protein embedding, the 1D sequence of embeddings can include the amino acid embeddings of the protein embedding. As another example, when the first and second molecule embeddings include a ligand embedding, the 1D sequence of embeddings can include the atom embeddings of the ligand embedding. As another example, when the first and second molecule embeddings include a nucleic acid embedding, the 1D sequence of embeddings can include the nucleotide embeddings of the nucleic acid embedding.

The embeddings included in the 1D sequence of embeddings can be ordered in any appropriate way, e.g., the 1D sequence of embeddings can be ordered to have the component embeddings of the first molecule embedding followed by the component embeddings of the second molecule embedding, or to have the component embeddings of the second molecule embedding followed by the component embeddings of the first molecule embedding. The length of the 1D sequence of embeddings can be a sum of: (i) the number of component embeddings in the first molecule embedding, and (ii) the number of component embeddings in the second molecule embedding. The 1D sequence of embedding can be represented by data having dimensionality NumTokens×d, where NumTokens is given by the sum of: (i) the number of component embeddings in the first molecule embedding, and (ii) the number of component embeddings in the second molecule embedding, and d is a positive integer value defining the number of channel dimensions in each component embedding.

The system processes the 1D sequence of embeddings to generate data defining a two-dimensional (2D) array of embeddings (216). The 2D array of embeddings can be represented by data having dimensionality NumTokens×NumTokens×d′ where (as above) NumTokens is given by the sum of: (i) the number of component embeddings in the first molecule embedding, and (ii) the number of component embeddings in the second molecule embedding, and d′ is a positive integer value defining the number of channel dimensions in each embedding of the 2D array of embeddings (d′ can be equal to d, i.e., the number of channel dimensions in each component embedding). The system can generate the 2D array of embeddings from the 1D sequence of embeddings in any of a variety of ways. For instance, the system can generate the 2D array of embeddings as a result of an element-wise outer product of the 1D sequence of embeddings with itself. As another example, the system can generate the 2D array by an appropriate 2D concatenation operation, e.g., where the embedding at each position (i, j) in the 2D array of embeddings is generated by concatenating: (i) the embedding at position i, and (ii) the embedding at position j, in the 1D sequence of embeddings (where indices i, j∈{1, . . . . N}, where N is the length of the 1D sequence of embeddings).

Each embedding in the 2D array of embeddings can be a component-component embedding of a first component embedding from the first molecule embedding and a second component embedding from the second molecule embedding.

For example, when the 1D sequence of embeddings includes a protein embedding of a protein and a ligand embedding of a ligand, each embedding of the 2D array of embeddings can be: (i) an atom—atom embedding, or (ii) an amino acid—amino acid embedding, or (iii) an amino acid—atom embedding. Each atom—atom embedding can be derived from a pair of atom embeddings representing atoms in the ligand. Each amino acid—amino acid embedding can be derived from a pair of amino acid embeddings representing amino acids in the protein. Each amino acid—atom embedding can be derived from a pair of embeddings that includes an amino acid embedding representing an amino acid in the protein and an atom embedding representing an atom in the ligand.

The system processes the 2D array of embeddings by a set of neural network layers of the fusion neural network to generate an updated 2D array of embeddings defining the joint molecule embedding (218). The updated 2D array of embeddings can have the same dimensionality as the original 2D array of embeddings, e.g., NumTokens×NumTokens×d′ (where NumTokens and d′ are defined as above). To generate the updated 2D array of embeddings, the fusion neural network can process the 2D array of embeddings using a sequence of one or more self-attention blocks. Each self-attention block can be configured to receive the current 2D array of embeddings as an input, to update the current 2D array of embeddings by one or more self-attention operations (e.g., single-head or multi-head query-key-value (QKV) or other self-attention operations), and to provide the updated 2D array of embeddings to a subsequent neural network layer (e.g., to another self-attention block, or to an output layer of the fusion neural network).

The self-attention blocks of the fusion neural network can implement any appropriate self-attention operations. A few examples of self-attention operations that can be implemented by self-attention blocks of the fusion neural network are described next.

In some implementations, one or more of the self-attention blocks of the fusion neural network implement “row-wise” or “column-wise” self-attention over the current 2D array of embeddings (i.e., that is provided as an input to the self-attention block). In a row-wise self-attention operation, a self-attention layer updates each given embedding in the 2D array of embeddings using a self-attention operation over only embeddings located in the same row as the given embedding in the 2D array of embeddings. In a column-wise self-attention operation, a self-attention block updates each given embedding in the 2D array of embeddings using a self-attention operation over only embeddings located in the same column as the given embedding in the 2D array of embeddings.

In some implementations, one or more of the self-attention blocks of the fusion neural network implement triangle self-attention operations. Example implementations of triangle self-attention operations are described in Abramson et al., “Accurate structure prediction of biomolecular interactions with AlphaFold 3,” Nature, Vol 630, 8 May 2024, and in Jumper et al., “Highly accurate protein structure prediction with AlphaFold,” Nature, Vol 596, 26 Aug. 2021. A triangle self-attention operation can be one that performs an attention operation on a triplet of embeddings, in particular between one embedding and a pair of other embeddings, more particularly using a triangle concept to provide consistency of respective relationships between the embedding and each of the other embeddings.

In some implementations, one or more of the self-attention blocks of the fusion neural network implement a full self-attention operation over the current 2D array of embeddings, e.g., by updating each embedding in the 2D array of embeddings using attention over the entire 2D array of embeddings.

The fusion neural network can include other neural network layers, i.e., in addition to the sequence of self-attention blocks, e.g., other neural network layers (such as fully connected layers or normalization layers) that are interleaved among the self-attention blocks. The fusion neural network can also include features such as skip connections, e.g., to implement residual blocks in the fusion neural network.

In some implementations, the fusion neural network can include multiple sub-networks (e.g., processing layers, blocks, etc.) that are distributed across multiple computing devices. In particular, the fusion neural network can be configured to generate the 2D array of embeddings by performing data sharding across the multiple computing devices, as described by Lepikhin et al. in “GShard: Scaling Giant Models with Conditional Computation and Automatic Sharding”. For example, to reduce or parallelize the computational cost of generating the 2D array of embeddings, the fusion neural network can process different sub-sets (e.g., micro-batches) of embeddings from the 1D sequence of embeddings as different shards that are distributed across the computational devices for the fusion neural network.

The system outputs the 2D array of embeddings generated by the fusion neural network as the joint molecule embedding (220). The system can provide the joint molecule embedding generated by the fusion neural network, e.g., for generating one or more predictions of the first molecule and the second molecule.

FIG. 2C illustrates operations performed by a molecule prediction system to generate a joint molecule embedding. The system processes molecule data 102 to generate a sequence of component embeddings 232-A representing a first molecule and a sequence of component embeddings 232-B representing a second molecule. Each of the component embeddings of the sequences of component embeddings 232-A and 232-B characterizes a molecule component (e.g., an atom, an amino acid) of a respective molecule represented by the molecule data 102.

As an example, the molecule data 102 can represent a protein and the sequence of component embeddings 232-A (resp., the sequence of component embeddings 232-B) can be a protein embedding for the protein that includes a sequence of amino acid embeddings (e.g., one for each amino acid in each amino acid sequence of the protein). As another example, the molecule data 102 can represent a ligand and the sequence of component embeddings 232-A (resp., the sequence of component embeddings 232-B) can be a ligand embedding for the ligand that includes a sequence of atom embeddings (e.g., one for each atom in the ligand).

The molecule prediction system concatenates the sequence of component embeddings 232-A and the sequence of component embeddings 232-B into a 1D sequence of embeddings, and then transforms the 1D sequence of embeddings (e.g., by an outer product operation 234) into a 2D array of embeddings 236. The 2D array of embeddings 236 can include component-component embeddings, where each component-component embedding is derived from a pair of component embeddings from the 1D sequence of embeddings. For example, the 2D array of embeddings 236 can include: (i) component-component embeddings 238-AA, each derived from a first embedding from the sequence of component embeddings 232-A and a second embedding from the sequence of component embeddings 232-A, (ii) component-component embeddings 238-AB, each derived from a first embedding from the sequence of component embeddings 232-A and a second embedding from the sequence of component embeddings 232-B, (iii) component-component embeddings 238-BA, each derived from a first embedding from the sequence of component embeddings 232-B and a second embedding from the sequence of component embeddings 232-A, and (iv) component-component embeddings 238-BB, each derived from a first embedding from the sequence of component embeddings 232-B and a second embedding from the sequence of component embeddings 232-B.

As a further example, when the sequence of component embeddings 232-A is a protein embedding representing a protein and the sequence of component embeddings 232-B is a ligand embedding representing a ligand, the 2D array of embeddings can include: (i) amino acid—amino acid embeddings (e.g., the component-component embeddings 238-AA), (ii) atom—atom embeddings (e.g., the component-component embeddings 238-BB), and (iii) amino acid—atom embeddings (e.g., the component-component embeddings 238-AB and 238-BA). Each atom—atom embedding can be derived from a pair of atom embeddings representing atoms in the ligand. Each amino acid—amino acid embedding can be derived from a pair of amino acid embeddings representing amino acids in the protein. Each amino acid—atom embedding can be derived from a pair of embeddings that includes an amino acid embedding representing an amino acid in the protein and an atom embedding representing an atom in the ligand.

The molecule prediction system can process the 2D array of embeddings 236 using a sequence of one or more self-attention blocks (e.g., that implement row-wise attention, or column-wise attention, or triangle self-attention, or full self-attention) to generate a joint molecule embedding that jointly represents the molecules represented by the molecule data 102.

FIG. 3 is a flow diagram of an example process 300 for generating a prediction for molecules using a molecule prediction system. For convenience, the process 300 will be described as being performed by a system of one or more computers located in one or more locations. For example, a molecule prediction system, e.g., the molecule prediction system 100 of FIG. 1, appropriately programmed in accordance with this specification, can perform the process 300.

The system obtains molecule data characterizing molecules (302). As described above, the molecule data can characterize a variety of molecules (e.g., proteins, ligands, etc.). For example, the molecule data can characterize a first protein and a second protein, and the system can generate a prediction for a protein-protein complex that includes the first protein and the second protein. As another example, the molecule data can characterize a protein and a ligand, and the system can generate a prediction for a protein-ligand complex that includes the protein and the ligand.

As an example, the molecule data can include protein data that includes any appropriate data characterizing a protein, e.g., data defining one or more amino acid sequences of the protein, or data defining an MSA for the protein, or data characterizing a respective structure of each of one or more “template” proteins, or a combination thereof. The structure of a template protein can be represented in any appropriate manner, e.g., by a contact map, or by data defining a respective 3D spatial position of each atom in the template protein. Optionally, the protein data can exclude any data that directly defines the 3D structure of the protein, e.g., the 3D spatial locations of the atoms or amino acid residues in a 3D conformation of the protein.

As another example, the molecule data can include ligand data that includes any appropriate data characterizing a ligand. For instance, the ligand data can include a textual representation of one or more of: a chemical structure of the ligand (e.g., the arrangement of atoms and bonds in the ligand), the atom types in the ligand and their connectivity, the chirality of the bonds in the ligand, or any functional groups (e.g., hydroxyl groups, amino groups, carboxyl groups, and so forth) included in the ligand. The textual representation of the ligand can include, e.g., a simplified molecular-input line-entry system (SMILES) string characterizing the ligand. As another example, the ligand data can include a representation of the ligand by way of graph data representing a graph, e.g., where the nodes in the graph represent atoms in the ligand and the edges in the graph represent bonds between atoms in the ligand. Optionally, the ligand data can exclude any data that directly defines the 3D structure of each ligand, e.g., the 3D spatial locations of the atoms in a 3D conformation of the ligand.

The system can receive the molecule data from any appropriate source, e.g., from a user or from another system, by way of an appropriate interface, e.g., an application programming interface (API), a user interface (e.g., a graphical user interface), and so on.

The system generates a joint molecule embedding using an embedding neural network (304). The embedding neural network processes the molecule data to generate a joint molecule embedding that jointly represents the molecules represented by the molecule data. The joint molecule embedding can be a 2D array that jointly represents the molecules represented by the molecule data.

The embedding neural network can be jointly trained with a plurality of prediction neural networks to generate joint molecule embeddings. An example process of training the embedding neural network with a prediction neural network is described in more detail below with reference to FIG. 4.

The system processes the joint molecule embedding using a prediction machine learning model to generate a prediction for the molecules represented by the obtained molecule data (306). The prediction machine learning model is configured to process the joint molecule embedding to generate the prediction for the molecules.

The prediction machine learning model can, for example, be a prediction neural network with any appropriate neural network architecture that enables the prediction machine learning model to perform its described functions. In particular, the prediction neural network can include any appropriate types of neural network layers (e.g., fully connected layers, convolutional layers, attention layers, etc.) in any appropriate number (e.g., 5 layers, 10 layers, or 20 layers) and connected in any appropriate configuration (e.g., as a directed graph of layers). In particular, the prediction network can be one of the plurality of prediction neural networks jointly trained with the embedding neural network.

As an example, the prediction machine learning model can be a property prediction neural network. The property prediction neural network can process the joint molecule embedding and generate a value of a predicted property (e.g., a binding affinity, a binding energy, etc.) for the molecules represented by the obtained molecule data. An example property prediction machine learning model is described in more detail below with reference to FIG. 5A-FIG. 5D.

As another example, the prediction machine learning model can be a structure prediction neural network. The structure prediction neural network can process the joint molecule embedding and generate predicted 3D structure (e.g., a predicted joint 3D structure) for the molecules represented by the obtained molecule data. An example structure prediction machine learning model is described in more detail below with reference to FIG. 6A-FIG. 6B.

As another example, the prediction machine learning model can be a molecule design neural network. When the obtained molecule data characterizes a protein, the molecule design neural network can process the joint molecule embedding and generate data characterizing a ligand designed to bind with the protein represented by the obtained molecule data. An example molecule design neural network is described in more detail below with reference to FIG. 7A-FIG. 7F.

FIG. 4 is a flow diagram of an example process 400 for jointly training an embedding neural network with a prediction machine learning model. For convenience, the process 400 will be described as being performed by a system of one or more computers located in one or more locations. For example, a molecule prediction system, e.g., the molecule prediction system 100 of FIG. 1, appropriately programmed in accordance with this specification, can perform the process 400.

The prediction neural network and the embedding neural network can be jointly trained on multiple training examples. Each training example corresponds to a respective collection of molecules for the training example and includes data defining: (i) a training input to the molecule prediction system for the training example, and (ii) a target output of the prediction neural network for the training example. The training input to the molecule prediction system includes molecule data for molecules of the training example (e.g., protein data characterizing a protein for the training example, ligand data characterizing a ligand for the training example, etc.). The target output to the molecule prediction system can include a target prediction for the training example.

For each training example, the system processes the training input of the training example using the embedding neural network to generate a joint molecule embedding of the molecules for the training example (402).

The system processes the joint molecule embedding for the training example using the prediction neural network to generate a prediction for the training example (404).

The system backpropagates gradients of an objective function through the prediction neural network and into the embedding neural network (406). The objective function can be any appropriate objective function for the prediction neural network.

For example, when the prediction neural network is a property prediction neural network, the objective function can measure a discrepancy between: (i) a target property score specified by the training example, and (ii) a predicted property score generated using the embedding neural network and the property prediction neural network for the training example. The objective function can, for example, measure an error (e.g., a root mean square deviation (RMSD), or a mean absolute error (MAE), or a mean squared error (MSE)) between: (i) the predicted property score for the training example, and (ii) the target property score for the training example.

As another example, when the prediction neural network is a structure prediction neural network, the objective function can measure an error (discrepancy) between: (i) a predicted structure of a molecule or molecule complex that is generated by the structure prediction neural network, and (ii) a target structure of the molecule or molecule complex that is specified by the training example. The objective function can measure the error, e.g., using a root-mean-square deviation (RMSD) measure, or a global distance test (GDT) measure, or a coordinate-based loss function, or using any other appropriate error measure. As a particular example, the objective function can measure a frame-aligned prediction error (FAPE) as described by Jumper et al. in “Highly accurate protein structure prediction with AlphaFold,” Nature, Vol 596, 26 Aug. 2021.

The system can determine gradients of the objective function with respect to the (learnable) parameters, e.g. weights, of the prediction neural network and the embedding neural network using backpropagation. The system can then update the current values of the parameters of the embedding neural network and the prediction neural network using the gradients, e.g., by the update rule of an appropriate gradient descent optimization algorithm, e.g., RMSprop or Adam.

FIG. 5A is a block diagram of an example property prediction neural network 500 of a molecule prediction system, e.g., the molecule prediction system 100 of FIG. 1.

As described above, the molecule prediction system 100 can process molecule data 102 characterizing one or more molecules using an embedding neural network 108 to generate a joint molecule embedding 106 that jointly represents the molecules represented by the molecule data 102.

The molecule prediction system 100 can include the property prediction neural network 500. The property prediction neural network can be configured to process the joint molecule embedding 106 to generate a property score 502 that defines a predicted property of the molecules represented by the molecule data 102.

The property score 502 can characterize any appropriate property of the molecules represented by the molecule data 102. A few examples of possible property scores are described next.

In one example, the property score can define a likelihood of occurrence of a binding event that involves the molecules represented by the molecule data 102.

In another example, the property score can define a binding affinity of the molecules represented by the molecule data 102, e.g., as measured using a particular binding affinity assay, as will be described in more detail below. The binding affinity of the molecules characterizes a strength or degree of attraction between the molecules when they interact to form a complex.

In another example, the property score can define a likelihood that one of the molecules represented by the molecule data 102 (e.g., a ligand) is an agonist for another molecule represented by the molecule data 102 (e.g., a protein).

In another example, the property score can define a likelihood that one of the molecules represented by the molecule data 102 (e.g., a ligand) is an antagonist for another molecule represented by the molecule data 102 (e.g., a protein).

In another example, the property score can characterize any appropriate predicted downstream effect of one of the molecules represented by the molecule data 102 (e.g., a ligand) acting on another molecule represented by the molecule data 102 (e.g., a protein). For instance, when the molecule data 102 represents a protein and a ligand, the property score can define a predicted potency of the ligand in acting on the protein, e.g., as measured by a half maximal effective concentration (EC50) of the ligand when acting on the protein. In another example, the property score can define a predicted inhibitory effect of the ligand when acting on the protein, e.g., as measured by a half maximal inhibitory concentration (IC50) of the ligand when acting on the protein.

Optionally, the system can generate multiple property scores, i.e., instead of a single property score. For instance, the system can generate any combination of two or more of the example property scores that are described above.

The property prediction neural network 500 can process the joint molecule embedding 106 using prediction layers 504 to generate the property score 502. In general, the prediction layers 504 can have any neural network architecture appropriate for processing the joint molecule embedding 106 to generate the property score 502 (e.g., including multi-layer perceptron layers, recurrent layers, convolutional layers, attention layers, etc.).

As a particular example, in some implementations, the predictions layers 504 of the property prediction neural network 500 can be a graph neural network configured to process a graph representation of the molecules (e.g., a graph representation generated using the joint molecule embedding 106) to generate the property score 502.

In some implementations, the prediction layers 504 can process a predicted joint 3D structure 506 for the molecules as part of generating the property score 502. The property prediction neural network 500 can obtain the predicted joint 3D structure 506 by a variety of methods. As an example, when the molecule prediction system 100 includes a structure prediction neural network, the property prediction neural network 500 can obtain the predicted joint 3D structure 506 as generated by the structure prediction neural network processing the joint molecule embedding 106. As another example, the property prediction neural network 500 can include a generative model 508 configured to generate the predicted joint 3D structure 506 by processing the joint molecule embedding 106.

For example, the property prediction neural network 500 can generate the predicted property 502 by generating the predicted joint 3D structure 506 for the molecules using the generative model 508, generating a graph representation of the 3D structure for the molecules, and then processing the graph representation using a graph neural network. An example process of generating the predicted property 502 using a graph neural network is described in more detail below with reference to FIG. 5B.

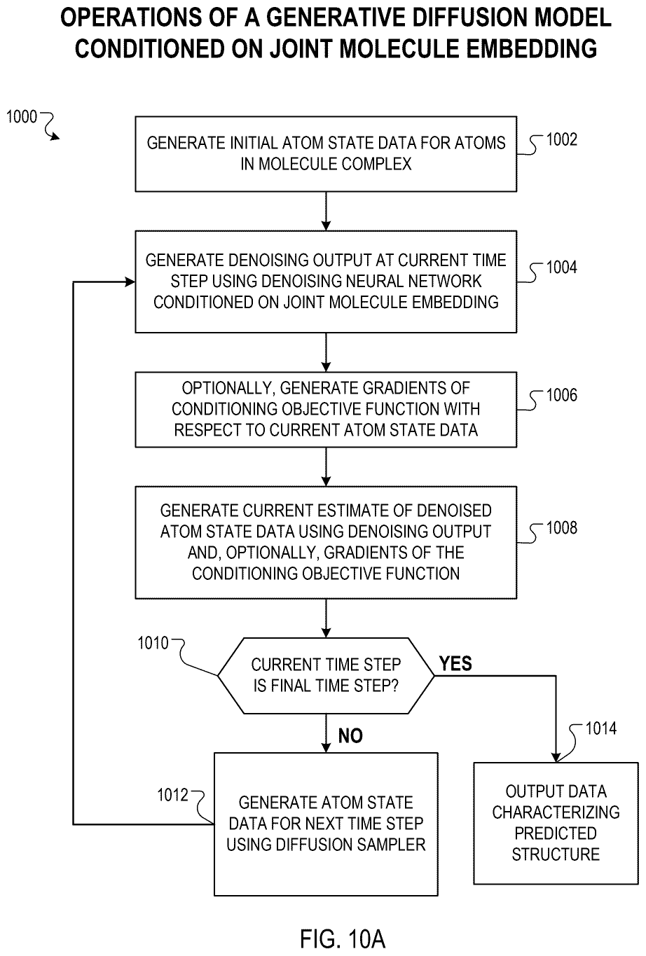

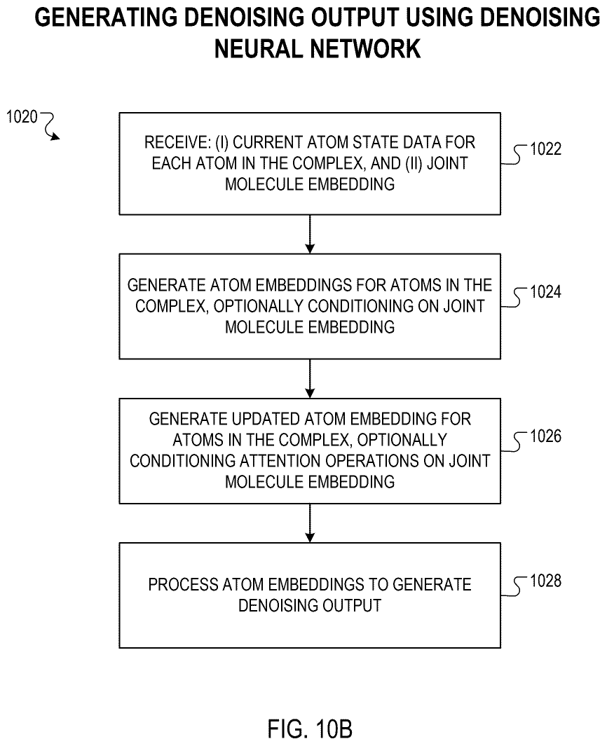

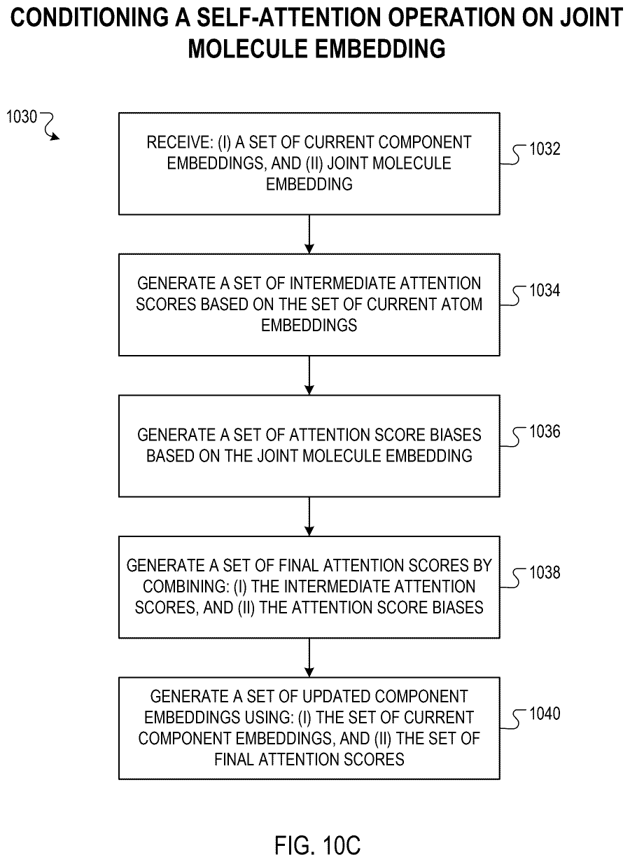

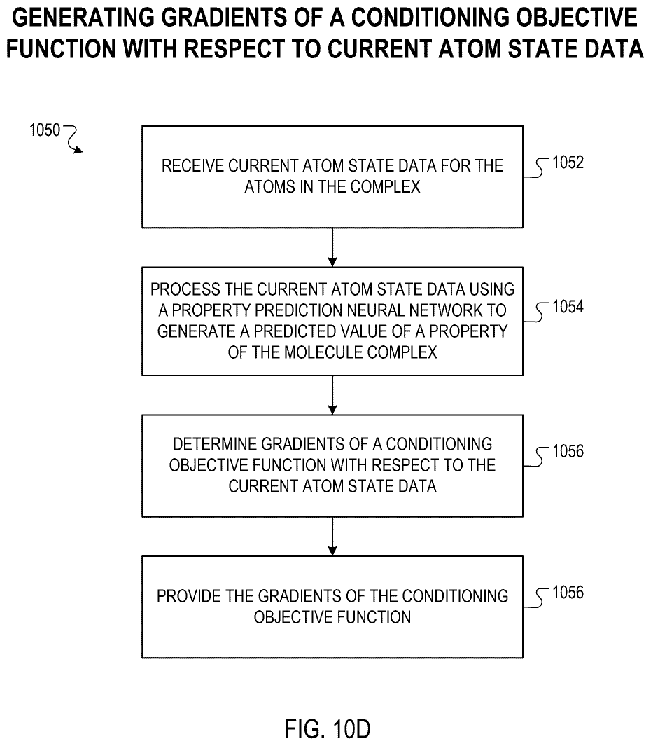

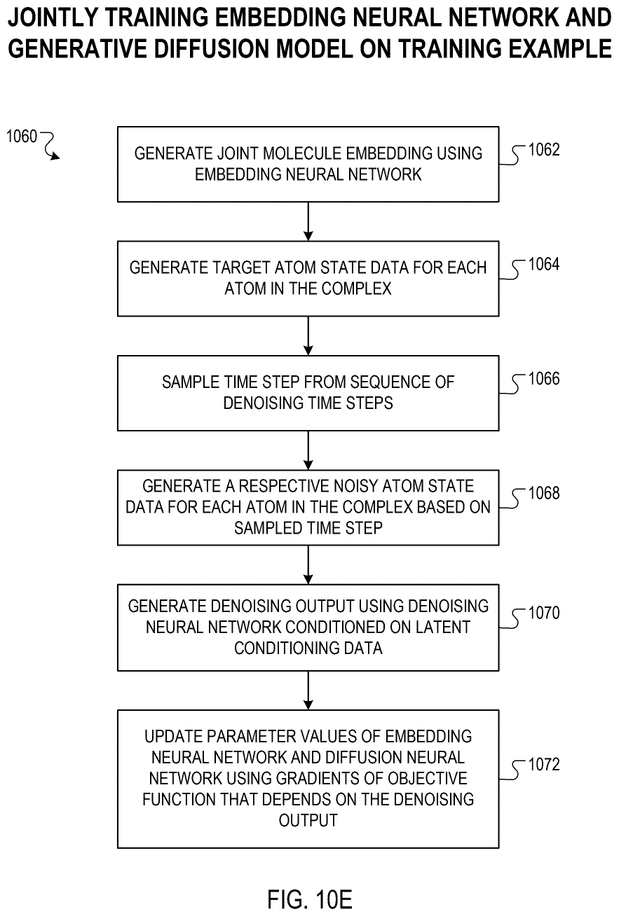

In some implementations, the generative model 508 is implemented as a generative diffusion model, and the property prediction neural network 500 generates the predicted property 502 by iteratively denoising the 3D spatial positions of the atoms in the complex along with data defining the property score 502. These implementations are described in more detail below with reference to FIG. 10A-10D.

The embedding neural network 112 can be jointly trained with a generative model 508 of the property prediction neural network 500. The generative model 508, when conditioned on the joint molecule embedding 106, is configured to generate one or more predicted joint 3D structures 506 of the complex. The predicted joint 3D structure 506 defines a respective predicted 3D spatial location of each atom in the molecules, i.e., of each atom in the molecules represented by the molecule data 102. In particular, the predicted joint 3D structure can define a structure of a complex in which one of the molecules represented by the molecule data 102 (e.g., a ligand) is bound to a binding site on the other molecule represented by the molecule data 102 (e.g., a protein).

The generative model 508 can be any appropriate conditional generative model. More specifically, the generative model 508 can be any appropriate model that, when conditioned on the joint molecule embedding 106, can generate samples from a distribution over a space of possible joint 3D structures of the complex. For instance, the generative model 508 can be implemented as a generative diffusion model, or a generative adversarial neural network (GAN) model, or a flow-based neural network model (normalizing flow model), and so forth.

Optionally, the generative model 508 can generate multiple distinct predicted joint 3D structures of the molecules. In particular, the generative model 508 can generate multiple samples from the distribution over the space of possible joint 3D structures of the molecules. Differences between the predicted joint 3D structures generated by the generative model 508 can reflect both uncertainty in the predicted structure and also the various structural modes of a complex that includes the molecules represented by the molecule data 102.

The molecule prediction system 100 can jointly train the embedding neural network 112 and the generative model 508 on a set of training data using an appropriate machine learning training technique. The training data can include a set of training examples, where each training example corresponds to a complex of a protein and a ligand, e.g., where the ligand is bound to a binding site on the protein. Each training example can include a training input that characterizes a training protein and a training ligand, and (ii) a target output based on a joint 3D structure of the training protein and the training ligand.

For example, the machine learning training technique can include processing the training input of the training example using the embedding neural network 112 to generate a joint molecule embedding of the training protein and the training ligand. The property prediction system 100 can then process the joint molecule embedding of the training protein and the training ligand using the generative model 508 to generate a predicted output characterizing a predicted joint 3D structure of the training protein and the training ligand of the training example. The molecule prediction system 100 can backpropagate gradients of an objective function through the generative model 508 and into the embedding neural network 112. The objective function can measure a discrepancy between the target output specified by the training example and the predicted output generated by the embedding neural network 112 and the generative model 508 for the training example. An example process for jointly training the embedding neural network 112 and a generative diffusion model (parametrized by a denoising neural network) is described in more detail with reference to FIG. 10E.

In some examples, when the molecule data 102 characterizes a protein and a ligand, the property prediction neural network 500 generates a binding affinity score that defines the predicted binding affinity of the protein and the ligand by conditioning the generation of the binding affinity score on data specifying a type of binding affinity assay. The binding affinity score can define the predicted binding affinity of the protein and the ligand as measured by the specified type of binding affinity assay. The binding affinity score can correspond to any appropriate type of binding affinity assay, i.e., any appropriate experimental technique for quantitatively measuring binding affinity. The type of binding affinity assay can be, for example, a surface plasmon resonance (SPR) assay, or an isothermal titration calorimetry (ITC) assay, or a fluorescence-based assay (e.g., a fluorescence-based polarization (FP) assay), or an enzyme-linked immunosorbent assay (ELISA), or a radioligand binding assay, or a bioluminescence resonance energy transfer (BERT) assay, etc. A user of the system can specify the type of assay corresponding to the binding affinity to be generated by the system, e.g., by way of a user interface or application programming interface (API) made available by the system. Example mechanisms by which the system can condition the generation of the binding affinity score on data specifying a type of binding affinity assay are described in more detail below.

Providing a mechanism for conditioning on the type of binding affinity assay can increase the amount of training data available for training the system, e.g., because the system can be trained on binding affinity training data associated with a variety of different binding affinity assays rather than being limited to only training data associated with a single binding affinity assay.

FIG. 5B is a flow diagram of an example process 520 for generating a predicted property score for a molecule complex using a graph neural network. For convenience, the process 520 will be described as being performed by a system of one or more computers located in one or more locations. For example, a molecule prediction system, e.g., the molecule prediction system 100 of FIG. 1, appropriately programmed in accordance with this specification, can perform the process 520.

The system generates a joint molecule embedding using an embedding neural network (522). The embedding neural network processes molecule data representing a first molecule and a second molecule (e.g., a protein and a ligand, a first protein and a second protein, etc.) to generate a joint molecule embedding that jointly represents the molecules. The joint molecule embedding can be a 2D array that represents the molecules.

In some implementations, the system generates a predicted joint 3D structure of a molecule complex of the molecules (e.g., a protein-ligand complex, a protein-protein complex, etc.) using a generative model and when the generative model is conditioned on the joint molecule embedding (524). The predicted joint 3D structure of the molecule complex can define a respective predicted three-dimensional spatial location of each atom in the molecules. An example process for generating a predicted 3D structure of a molecule complex using a generative diffusion model is described with reference to FIG. 10A-FIG. 10D.

The system generates data defining an input graph based on the joint molecule embedding for the molecules (526). The input graph includes a set of nodes and a set of edges, where each edge in the set of edges connects a respective pair of nodes in the set of nodes. When the system generates a predicted joint 3D structure of a molecule complex for the molecules, the system can generate the input graph to represent at least a portion of the predicted joint 3D structure of the molecule complex. An example process for generating a graph representing a 3D structure of a molecule complex is described with reference to FIG. 5C.

The system processes the input graph representing at least the portion of the predicted joint 3D structure of the molecule complex using a graph neural network to generate the property score that defines the predicted property of the molecules (528). The graph neural network is configured to process a graph that includes edges and nodes to generate a property score.

The graph neural network can have any appropriate neural network architecture that enables the graph neural network to perform its described functions. In particular, the graph neural network can include any appropriate types of neural network layers (e.g., message passing layers, fully connected layers, convolutional layers, attention layers, etc.) in any appropriate number (e.g., 5 layers, 10 layers, or 20 layers) and connected in any appropriate configuration (e.g., as a directed graph of layers).

In some implementations, the graph neural network includes multiple message passing layers. The message passing layers can allow neighboring edges and nodes to exchange information and influence each other e.g., by using an update function. For example, the graph neural network can include 3 message passing layers that are stacked together. Each node in the graph can eventually incorporate information from nodes that are 3 steps away from it due to stacking 3 message passing layers together.

An example process for processing the input graph using the graph neural network to generate the predicted property score is described below with reference to FIG. 5D.

FIG. 5C is a flow diagram of an example process 530 for generating a graph based on a predicted 3D structure of a molecule complex. For convenience, the process 530 will be described as being performed by a system of one or more computers located in one or more locations. For example, a molecule prediction system, e.g., the molecule prediction system 100 of FIG. 1, appropriately programmed in accordance with this specification, can perform the process 530.

The system receives data defining a predicted 3D structure of the molecule complex (532). More specifically, the system receives a respective set of atom features for each atom in the complex. The set of atom features for an atom define the 3D spatial position of the atom in the molecule complex, and optionally, define one or more additional features of the atom, e.g., including one or more of: an element type of the atom, a partial charge of the atom, or a hybridization state of the atom. The predicted 3D structure of the complex may be generated by a generative model that is conditioned on a joint molecule embedding of molecules in the molecule complex.

The system instantiates a set of atom nodes for inclusion in the graph (534). Each atom node in the set of atom nodes represents a respective atom in the molecules of the molecule complex.

In some implementations, when the molecule complex includes a protein, the system generates a respective atom node representing each atom in the protein, i.e., so that every atom in the protein is represented by a respective atom node in the graph. In other implementations, the selects a proper subset of the atoms in the protein to be represented in the graph, e.g., rather than representing all the atoms in the protein in the graph. For instance, the system can identify a proper subset of the atoms in the protein as being included in the binding pocket to which another molecule of the molecule complex (e.g., a ligand, a protein, etc.) is bound, and then generate a respective atom node in the graph for only those atoms in the protein that are included in the binding pocket. In particular, the system can refrain from generating atom nodes in the graph representing the atoms in the protein that are outside the binding pocket. The system can identify the binding pocket in the protein in any of variety of possible ways. For instance, the system can determine that any atom in the protein that is within a threshold distance (e.g., 2 Angstroms) of at least one atom in another molecule of the molecule complex (i.e., in the 3D structure of the molecule complex) is included in the binding pocket of the protein.

Generating a graph that represents only a proper subset (i.e. less than all) of the atoms in a protein, e.g., only the atoms in the binding pocket of the protein, can reduce consumption of computational resources during processing of the graph by the graph neural network. Further, training the graph neural network to predicting molecule complex properties based on graphs that represent only the binding pocket of a protein (i.e., as opposed to the whole protein) can improve the generalization performance of the graph neural network, e.g., by reducing the likelihood of the graph neural network overfitting the training data by learning to memorize irrelevant features of the protein that are distant from the binding pocket.

Optionally, the system generates a set of super nodes for inclusion in the graph. In contrast to atom nodes, which represent individual atoms in the complex (as described above), a super node represents a higher-level entity in the complex such as an amino acid or a structural motif.

More specifically, the system can generate a respective super node representing some or all of the amino acids in a protein of the molecule complex. For instance, the system can generate respective super node for each amino acid in the entire protein, or the system can generate a respective super node for only those amino acids in the protein that are included in the binding pocket of the protein. (The system can determine that an amino acid is included in the binding pocket of the protein, e.g., if the amino acid includes at least one atom that is in the binding pocket of the protein).

Further, the system can generate a respective super node representing each structural motif in a ligand of the molecule complex. More specifically, the system can partition the atoms in the ligand into multiple groups of atoms that each represent a respective structural motif from a set of possible structural motifs. A structural motif refers to a specific combination and arrangement of atoms in a molecule, and the set of possible structural motifs can include one or more of: aromatic rings, chelating groups, sulfonamides, and so forth. The system can generate a respective super node representing each structural motif in the ligand. Thus, for instance if the ligand includes 100 atoms that are partitioned into 10 structural motifs, the system can generate 10 super nodes, each of which represents a respective structural motif in the ligand.

The system instantiates a set of edges in the graph (536). For each pair of atom nodes in the graph, the system can determine that the pair of atom nodes should be connected by an edge in the graph if the corresponding pair of atoms represented by the pair of atom nodes are separated by less than a threshold distance (e.g., 2 Angstroms, or 5 Angstroms, or 10 Angstroms) in the 3D structure of the complex.

The system can instantiate edges that fully connect any super nodes in the graph, i.e., such that every pair of super nodes in the graph is connected by an edge in the graph. In particular, the system can instantiate edges that connect: (i) each super node representing an amino acid in the molecule complex to each other super node representing an amino acid in the molecule complex, (ii) each super node representing an amino acid to each super node representing a structural motif in the molecule complex, and (iii) each super node representing a structural motif in the molecule complex to each other super node representing a structural motif in the molecule complex.

For each atom node that represents a respective atom in a protein of the molecule complex, the system can instantiate an edge that connects the atom node to a super node representing an amino acid that includes the atom.

For each atom node that represents a respective atom in a ligand of the molecule complex, the system can instantiate an edge that connects the atom node to a super node representing a structural motif that includes the atom.

The super nodes in the graph facilitate long range propagation of information across the graph as the graph is being processed by the message passing layers of the graph neural network. More specifically, the graph can include a large number of atom nodes, and only those atom nodes that are in separated by less than a threshold spatial distance in the 3D structure of the complex are connected by an edge. Thus the operations of the message passing layers, if reliant only on propagating information along edges connecting the atom nodes, may be unable efficiently to propagate information between spatially distant atoms in the complex. The system can address this issue by the inclusion of super nodes in the graph because the super nodes can greatly reduce the maximum “path length” between atom nodes in the graph to, e.g., such that the maximum path length between nodes in the graph is three. (The “path length” between a pair of atom nodes can refer to the minimum number of edges in a path connecting the pair of atom nodes in the graph). The inclusion of super nodes can thus allow the graph neural network to rapidly propagate information between all the atom nodes in the graph, even between those atom nodes that represent spatially distant atoms in the complex. A super node can be processed in the same way as other nodes of the graph, as described further below, although it represents different information.

The system associates a respective set of features with each node in the graph (538). For instance, for each atom node in the graph, the system can associate a set of features with the atom node including one or more of: a feature indicating whether the atom node is included in a protein of the molecule complex or in a ligand of the molecule complex; a feature defining a 3D spatial position of the atom in the molecule complex; a feature defining an elemental type of the atom; a feature defining a partial charge of the atom; a feature defining a hybridization state of the atom; and so forth. As another example, for each super node in the graph, the system can associate a set of features with the super node including one or more of: a feature indicating whether the super node represents an amino acid in the molecule complex or a structural motif in the molecule complex; for super nodes representing amino acids in the molecule complex, a feature representing a type of the amino acid represented by the super node; for super nodes representing structural motifs in the molecule complex, a feature representing a type of the structural motif represented by the super node.

Optionally, the system can associate a set of features with each edge in the graph. For instance, the set of features associated with an edge between a pair of atom nodes can include a feature representing the spatial distance separating the pair of atoms represented by the pair of atom nodes in the 3D structure of the complex. As another example, the set of features associated with an edge can include a feature that classifies whether the edge connects: a pair of atom nodes; or a pair of super nodes; or an atom node to a super node.

FIG. 5D is a flow diagram of an example process 540 for processing a graph representing a 3D structure of a molecule complex using a graph neural network to generate a predicted property score. For convenience, the process 540 will be described as being performed by a system of one or more computers located in one or more locations. For example, a molecule prediction system, e.g., the molecule prediction system 100 of FIG. 1, appropriately programmed in accordance with this specification, can perform the process 540.

The system receives the graph representing the 3D structure of the molecule complex (542). The graph includes a set of nodes and a set of edges, where each edge in the set of edges connects a respective pair of nodes in the set of nodes. The graph can include atom nodes representing atoms in the complex, and optionally, super nodes that each represent a respective amino acid in the molecule complex or a respective structural motif in the molecule complex. Each node in the graph can be associated with a respective set of node features, and optionally, each edge in the graph can be associated with a respective set of edge features.

The system generates a respective embedding associated with each node in the graph using an encoder block of the graph neural network (544). More specifically, for each node in the graph, the encoder block processes the set of node features associated with the node to generate an embedding for the node. Optionally, for each edge in the graph, the encoder block can process the set of edge features associated with the edge to generate an embedding for the edge.

The system processes the node embeddings associated with the nodes in the graph, e.g. by a sequence of one or more message passing neural network layers (546). Each message passing neural network layer is configured to receive a set of current node embeddings associated with the nodes in the graph, to process the set of input node embeddings in accordance with values of a set of message passing neural network layer parameters and using operations that are conditioned on the topology of the graph, and to generate a respective updated node embedding associated with each node in the graph. For instance, for each node in the graph, a message passing layer can generate an updated node embedding for the node based on: (i) the current node embedding for the node, and (ii) the current node embeddings of any neighboring nodes of the node. (A “neighboring” node of a given node refers to node that is directly connected to the given node by an edge in the graph).

The first message passing neural network layer can receive the node embeddings generated by the encoder block of the graph neural network layer, as described at step 544. Each subsequent message passing neural network layer can receive the node embeddings generated as an output by the preceding message passing neural network layer.

Optionally, each message passing neural network layer can update the edge embeddings associated with the edges in the graph. For instance, a message passing neural network layer can update the edge embedding associated with an edge in the graph based on: (i) the edge embedding associated with the edge, and (ii) the node embeddings of the nodes that are connected by the edge. For example, a message passing neural network layer can perform an optional edge update operation to update the edge embeddings (e.g. using an edge update MLP), followed by a node update operation to update the node embeddings (e.g. using a node update MLP) that operates on a “message” between each pair of nodes that depends on the node embeddings of the nodes and the edge embedding their connecting edge.

The system processes the updated node embeddings generated by the final message passing neural network layer using an output block of the graph neural network to generate the predicted property score (548). For instance, the output block can pool (e.g., average, sum, or otherwise combine) the updated node embeddings (and, optionally, the updated edge embeddings) to generate a combined embedding, and process the combined embedding by a sequence of fully connected layers to generate the predicted property score.

In some implementations, the graph neural network can be configured to receive a network input that includes both: (i) the graph representing the 3D structure of the molecule complex, and (ii) data identifying a type of binding affinity assay. The graph neural network can then generate a predicted binding affinity associated with the type of assay specified in the network input. For instance, the output block of the graph neural network can jointly process the combined embedding and the data identifying the type of binding affinity assay to generate a predicted binding affinity associated with the type of assay specified in the network input.

FIG. 5E is a flow diagram of an example process 550 for training a graph neural network of a property prediction neural network. For convenience, the process 550 will be described as being performed by a system of one or more computers located in one or more locations. For example, a molecule prediction system, e.g., the molecule prediction system 100 of FIG. 1, appropriately programmed in accordance with this specification, can perform the process 550.

The system receives a set of training examples (552). Each training example corresponds to a respective molecule complex for the training example and includes data defining: (i) a training input to the graph neural network, and (ii) a target output of the graph neural network. The training input to the graph neural network includes graph data defining a graph representing at least a portion of the predicted joint 3D structure of the molecule complex. The target output of the graph neural network can include a target property score.

The system trains the graph neural network using the training examples to optimize an objective function (554). For each training example, the objective function can measure a discrepancy between: (i) the target property score specified by the training example, and (ii) the predicted property score generated by the graph neural network for the training example. The objective function can measure an error between: (i) the predicted property score, and (ii) the target property score. The objective function can be, e.g., a squared error objective function (e.g., when the target property score represents a continuous value such as a binding affinity) or a cross-entropy objective function (e.g., when the target property score represents a discrete value, e.g., for whether a binding event occurs, or a discrete value defining whether one molecule of the complex is an agonist or an antagonist for another molecule of the complex).

FIG. 6A is a block diagram of an example structure prediction neural network 600 of a molecule prediction system, e.g., the molecule prediction system 100 of FIG. 1.

As described above, the molecule prediction system 100 can process molecule data 102 characterizing one or more molecules using an embedding neural network 108 to generate a joint molecule embedding 106 that jointly represents the molecules represented by the molecule data 102.

The molecule prediction system 100 can include a structure prediction neural network 600 configured to process the joint molecule embedding 106 to generate a predicted joint 3D structure 602 of a molecule complex that includes the molecules represented by the molecule data 102. The predicted joint 3D structure 602 defines a respective predicted 3D spatial location of each atom in the complex, i.e., of each atom in the molecules represented by the molecule data 102.

As an example, when the molecule data 102 represents a protein and a ligand, the predicted joint 3D structure can define a structure of the complex where the ligand is bound to a binding site on the protein.

In particular, the structure prediction neural network 600 can be a generative model that, when conditioned on the joint molecule embedding 106, is configured to generate one or more predicted joint 3D structures 602 of the complex. The generative model can be any appropriate conditional generative model. More specifically, the generative model can any appropriate model that, when conditioned on the joint molecule embedding 106, can generate samples from a distribution over a space of possible joint 3D structures of the complex. For instance, the generative model can be implemented as a generative diffusion model, or a generative adversarial neural network (GAN) model, or a flow-based neural network model (normalizing flow model), and so forth. An example process for generating predicted joint 3D structures using a generative diffusion model is described detail with reference to FIG. 10A-FIG. 10D.

Optionally, the generative model can generate multiple distinct predicted joint 3D structures of the complex. In particular, the generative model can generate multiple samples from the distribution over the space of possible joint 3D structures of the complex. Differences between the predicted joint 3D structures 602 generated by the generative model can reflect both uncertainty in the predicted structure and also the various structural modes of a complex that includes the molecules represented by the molecule data 102.

An example process of generating a predicted joint 3D structure using a structure prediction neural network is described in more detail below with reference to FIG. 6B.

As described above, the embedding neural network 108 can be jointly trained with a plurality of prediction neural networks. The structure prediction neural network 600 can be one of the plurality of prediction neural networks jointly trained with the embedding neural network 108.

The molecule prediction system 100 can jointly train the embedding neural network 108 and the structure prediction neural network 600 on a set of training data using an appropriate machine learning training technique. For example, the training data can include a set of training examples, where each training example corresponds to a complex of a protein and a ligand, e.g., where the ligand bound to a binding site on the protein. An example process for jointly training the embedding neural network 108 and a generative diffusion model of the structure prediction neural network is described in more detail with reference to FIG. 10E.

After generating the predicted joint 3D structure 602, the molecule prediction system 100 can, e.g., store data defining the predicted joint 3D structure 110 in a memory, or transmit data defining the predicted joint 3D structure 602 over a data communication network, or provide data defining the predicted joint 3D structure 602 directly to a system that performs downstream processing based on the predicted joint 3D structure 602.

FIG. 6B is a flow diagram of an example process 610 for generating a predicted joint 3D structure for a molecule complex. For convenience, the process 610 will be described as being performed by a system of one or more computers located in one or more locations. For example, a molecule prediction system, e.g., the molecule prediction system 100 of FIG. 1, appropriately programmed in accordance with this specification, can perform the process 610.

The system generates a joint molecule embedding using an embedding neural network (612). The embedding neural network processes molecule data representing a first molecule and a second molecule (e.g., a protein and a ligand, a first protein and a second protein, etc.) to generate a joint molecule embedding that jointly represents the molecules. The joint molecule embedding can be a 2D array that represents the molecules.

The system generates a predicted joint 3D structure of a molecule complex of the molecules (e.g., a protein-ligand complex, a protein-protein complex, etc.) using a generative model and when the generative model is conditioned on the joint molecule embedding (614). The predicted joint 3D structure of the molecule complex can define a respective predicted three-dimensional spatial location of each atom in the molecules. An example process for generating a predicted 3D structure of a molecule complex using a generative diffusion model is described with reference to FIG. 10A-FIG. 10D.

In some implementations, the generative model can generate multiple distinct predicted joint 3D structures for the molecule complex.

FIG. 7A is a block diagram of an example molecule design neural network 700 of a molecule prediction system, e.g., the molecule prediction system 100 of FIG. 1. The molecule design neural network 700 can be, e.g., a ligand design neural network that is configured to perform a ligand design task.

As described above, the molecule prediction system 100 can process molecule data using an embedding neural network 108 to generate a joint molecule embedding 106. In particular, the molecule prediction system 100 can process target molecule data 702 characterizing at least a portion of a target molecule using the embedding neural network 108 to generate a joint molecule embedding 106 representing the target molecule.

The molecule prediction system 100 can include a molecule design neural network 700 configured to process the joint molecule embedding 106 to generate data defining a designed molecule 706 that is predicted to bind with the target molecule represented by the target molecule data 702. As an example, the target molecule data 702 can represent a target protein and the molecule design neural network 700 can generate data defining a designed ligand that is predicted to bind with the target protein. As another example, the target molecule data 702 can represent a target protein and the molecule design neural network 700 can generate data defining a designed protein that is predicted to bind with the target protein. As another example, the target molecule data 702 can represent a target nucleic acid and the molecule design neural network 700 can generate data defining a designed protein that is predicted to bind with the target nucleic acid.

The target molecule data 702 can include any appropriate data characterizing the target molecule. For example, when the target molecule is a protein, the target molecule data 702 can include, e.g., data defining one or more amino acid sequences of the protein, or data defining an MSA for the protein, or data characterizing a respective structure of each of one or more template proteins, or a combination thereof. In some cases, the target molecule data 702 can characterize a full molecule or molecule complex. In other cases, the target molecule data 702 can include data characterize only a portion of a target molecule, e.g., only a binding pocket of a target protein.

Optionally, molecule data processed by the molecule prediction system 100 can further include molecule design criteria 704 that specify: (i) a respective target (desired) value for each of one or more properties of the designed molecule (e.g., target molecule properties), or (ii) scaffolding data, or (iii) both. The embedding neural network 108 can process the target molecule data 702 and the molecule design criteria 704 to generate a joint molecule embedding 106 that jointly represents the target molecule represented by the target molecule data 702 and the molecule design criteria 704. Target molecule properties and scaffolding data are each described in more detail next (and throughout this specification).

The target molecule properties can specify a target (desired) value for any appropriate “global” or “atom-specific” properties of the designed molecule.

Global properties of a designed molecule can refer to properties that characterize the designed molecule as a whole rather than being specific to a single atom, and can include properties characterizing one or more of: a binding affinity of the designed molecule for the target molecule, absorption properties of the designed molecule, distribution properties of the designed molecule, metabolism properties of the designed molecule, excretion properties of the designed molecule, toxicity properties of the designed molecule, a number of rings (e.g., aromatic rings) in the designed molecule, a molecular weight of the designed molecule, a lipophilicity (logP) of the designed molecule, an ability of the designed molecule to donate or accept hydrogen bonds, a total polar surface area of the designed molecule, a number of rotatable bonds in the designed molecule, a number of chiral centers (stereocenters) in the designed molecule, a number of electrophilic (electron-accepting) centers in the designed molecule, a number of nucleophilic (electron-donating) centers in the designed molecule, and so forth.

Atom-specific properties of a designed molecule can refer to properties that relate to specific atoms in the designed molecule rather than the entire designed molecule, and can include properties characterizing one or more of: an elemental type of an atom (e.g., carbon, oxygen, nitrogen, and so forth), a hybridization state of an atom (e.g., sp hybridization, or sp² hybridization, or sp³ hybridization, or sp³d hybridization, or sp²d² hybridization, and so forth), a partial charge of an atom, and so forth.

The scaffolding data can specify target molecule scaffolding data, or designed molecule scaffolding data, or both. The target molecule scaffolding data can specify (at least) a portion of a 3D structure of the target molecule, in particular, by specifying a respective 3D spatial position of each of one or more atoms in the target molecule. The designed molecule scaffolding data can specify (at least) a portion of the 3D structure of the designed molecule, in particular, by specifying a respective 3D spatial position of each of one or more atoms in the designed molecule.

In some implementations, the scaffolding data can specify contact properties between the target molecule and the designed molecule. For example, the scaffolding data can specify that a particular molecule component (e.g., atom, group of atoms, amino acid, nucleic acid, etc.) of the target molecule is in contact (e.g., within a pre-defined threshold distance) with atoms of the designed molecule. As another example, the scaffolding data can specify that a particular molecule component of the target molecule is in contact with a particular molecule component of the designed molecule.

When the molecule prediction system 100 processes molecule design criteria 704, the system 100 can attempt to generate a designed molecule 706 that satisfies the molecule design criteria 704. For instance, if the molecule design criteria 704 include target molecule properties, then the system 100 can attempt to generate a designed molecule 706 that has the target molecule properties. As another example, if the molecule design criteria 704 include target molecule scaffolding data, then the system 100 can attempt to generate a designed molecule 706 that binds to the target molecule when the target molecule has the conformation defined by the target molecule scaffolding data. As another example, if the molecule design criteria 704 include designed molecule scaffolding data, then the system 100 can attempt to generate a designed molecule 706 that has the structure defined by the designed molecule scaffolding data.

In particular, the molecule prediction system 100 can have a set of system parameters (e.g., embedding neural network parameters and molecule design neural network parameters) that are configured through training (e.g., by a machine learning training technique) to encourage the generation of a designed molecule 706 that satisfies the molecule design criteria 704.

In some cases, a designed molecule 706 generated by the molecule prediction system 100 may satisfy all the molecule design criteria 704. However, in other cases, a designed molecule 706 generated by the molecule prediction system 100 may satisfy certain molecule design criteria 704 only approximately, or may entirely fail to satisfy certain molecule design criteria 704. This may occur, for instance, if the molecule design criteria 704 are mutually incompatible (e.g., if there does not exist a designed molecule that binds to the target molecule that simultaneously satisfies all the molecule design criteria 704), or if the design system parameters have not been trained on a sufficient amount or type of training data to enable the precise generation of a designed molecule that binds to the target and that satisfies all the molecule design criteria 704. In particular, providing molecule design criteria 704 to the molecule prediction system 100 increases the likelihood, but does not guarantee, that the molecule prediction system 100 will generate a designed molecule 706 that satisfies the molecule design criteria 704.

Generally, the molecule design criteria 704 do not specify the full chemical structure of the designed molecule 706. In particular, the molecule design criteria 704 leave undefined at least parts of the chemical structure of the molecule.

The output data defining the designed molecule 706 can include, for each atom in the designed molecule 706, respective atom state data for the atom that defines at least a respective 3D spatial position of the atom (i.e., in a complex that includes the target molecule). The atom state data for an atom can further include any other appropriate atom-specific properties of the atom, such as an elemental type of the atom, a hybridization state of the atom, a partial charge of the atom, and so forth. Here “each atom in the designed molecule” (and generally similar references herein) may exclude hydrogen atoms; or some or all hydrogen atoms may be included.

Optionally, the output data defining the designed molecule 706 can further include bond data that defines, for each pair of atoms in the designed molecule, whether the pair of atoms are connected by a bond. The bond data can further define one or more respective properties of each bond in the designed molecule, e.g., the type of the bond, e.g., single, double, or triple covalent bond, or ionic bond, or coordinate covalent bond, and so forth.

Where the molecule design neural network 700 is a ligand design neural network configured to perform a ligand design task, predicted ligand data generated by the ligand design neural network can be the output data defining the designed molecule 706, i.e. defining a predicted ligand that is predicted to bind to the target molecule, e.g. a protein, a nucleic acid, and so forth.

The molecule design neural network 700 can include a generative model 708 and, optionally, a bond prediction machine learning model 710, which are each described in more detail next (and throughout this specification).

The generative model 708, when conditioned on the joint molecule embedding 106, is configured to generate data defining the designed molecule 706, in particular, to generate respective atom state data for each atom in the designed molecule that defines at least a respective 3D spatial position of the atom (as described above). The generative model 708 can be any appropriate conditional generative model. More specifically, the generative model 708 can be any appropriate model that, when conditioned on the joint molecule embedding 106, can generate samples from a distribution over a space of possible designed molecules. For instance, the generative model 708 can be implemented as a generative diffusion model (e.g., as described by Ho et al. in “Denoising Diffusion Probabilistic Models”) or a related model such as a consistency model, or a generative adversarial neural network (GAN) model (e.g., as described by Goodfellow et al. in “Generative Adversarial Networks”), or a flow-based neural network model (e.g., a normalizing flow model as described by Rezende and Mohamed in “Variational Inference with Normalizing Flows”. Proc. ICML 2015), and so forth. An example process for generating designed molecules using a generative diffusion model is described detail with reference to FIG. 10A-FIG. 10D. Further details of a specific example of a molecule prediction system that can be used as molecule prediction system 100 are described in PCT/EP2025/058453 filed by the applicant on Mar. 27, 2025.

In some implementations, the generative model 708 can generate bond data that identifies bonds present in the designed molecule, and optionally, one or more properties of the bonds present in the designed molecule (as described above).

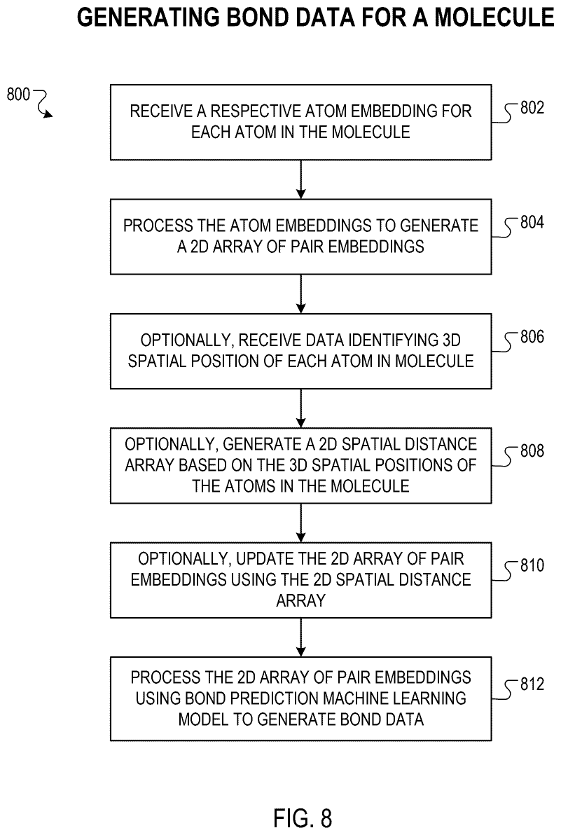

The bond prediction machine learning model 710 is configured to process a model input characterizing the designed molecule 706 to generate bond data that identifies bonds present in the designed molecule, and optionally, one or more properties of the bonds present in the designed molecule (as described above). An example of a bond prediction machine learning model 710 is described in more detail below with reference to FIG. 8.

In some implementations, rather than generating a single designed molecule 706, the molecule design neural network 700 can generate multiple distinct designed molecules 706. In particular, the molecule design neural network 700 can use the generative model 708 to generate multiple samples from the distribution over the space of possible designed molecules, each of which represents a respective different designed molecule (e.g. from different random initializations of the generative model) that is predicted to bind to the target molecule and to satisfy any molecule design criteria 704. Each of the designed molecules 706 generated by the molecule design neural network 700 can have different chemical structures and properties and can satisfy any molecule design criteria 704 to different degrees.

As described above, the embedding neural network 108 can be jointly trained with a plurality of prediction neural networks. The molecule design neural network 700 can be one of the plurality of prediction neural networks jointly trained with the embedding neural network 108.

In particular, the molecule prediction system 100 can jointly train the embedding neural network 108 and the generative model 708 to encourage that the generative model 708, when conditioned on a joint molecule embedding 106 generated by the embedding neural network 108 and that represents a target molecule and (optionally) molecule design criteria, generates designed molecules that bind to the target molecule and that satisfy the molecule design criteria. The molecule prediction system 100 can jointly train the embedding neural network 108 and the generative model 708 (and, optionally, the bond prediction machine learning model 710), on a set of training data using an appropriate machine learning training technique. The training data can include a set of training examples, where each training example corresponds to a complex of a target molecule and a designed molecule, e.g., where the designed molecule is bound to the target molecule. An example process for jointly training the embedding neural network 108 and the generative model 708 (and, optionally, the bond prediction machine learning model 710) is described in more detail with reference to FIG. 10E.

FIG. 7B is a flow diagram of an example process 720 for generating and screening designed molecules for binding to a target molecule. For convenience, the process 720 will be described as being performed by a system of one or more computers located in one or more locations. For example, a molecule prediction system, e.g., the molecule prediction system 100 of FIG. 1, appropriately programmed in accordance with this specification, can perform the process 720.

The system receives protein data characterizing at least a portion of a target molecule, and optionally, molecule design criteria (722). The molecule design criteria specify criteria to be satisfied by a designed molecule to be generated by the system. The molecule design criteria can include target molecule properties, or scaffolding data, or both. The target molecule properties can specify a respective target value for each of one or more properties of the designed molecule. The scaffolding data can include target molecule scaffolding data (specifying at least a portion of the 3D structure of the target molecule) or designed molecule scaffolding data (specifying at least a portion of the 3D structure of the designed molecule) or both.

The system can receive the protein data and the molecule design criteria from any appropriate source, e.g., from a user or from another system, by way of an appropriate interface, e.g., an application programming interface (API) or a user interface (e.g., a graphical user interface).

The system processes the target molecule data and any molecule design criteria using an embedding neural network to generate a joint molecule embedding representing the target molecule and any molecule design criteria (724). An example process of using the embedding neural network to generate a joint molecule embedding representing a target molecule and molecule design criteria is described in more detail below with reference to FIGS. 7C and 7D.

The system generates, using a generative model and while the generative model is conditioned on the joint molecule embedding representing the target molecule and any molecule design criteria, data defining a set of designed molecules that are each predicted to bind to the target molecule (726). The system generates each designed molecule in a manner that encourages the designed molecule to satisfy any molecule design criteria. As an example, the target molecule can be a protein and the molecule design criteria can be ligand design criteria that define desired characteristics of a predicted ligand. that binds to the protein. The embedding neural network and the ligand design neural network can have been jointly trained to optimize an objective function, e.g. loss, that measures a degree to which a ligand that binds to the protein, defined by the generated ligand data, satisfies the ligand design criteria. The set of designed molecules can include any appropriate number of designed molecules, e.g., 1, 10, 100, or 1000 designed molecules. An example process for generating data defining a designed molecule using a generative diffusion model is described in more detail with reference to FIG. 10A-FIG. 10D.

The system generates, for each designed molecule in the set of designed molecules, bond data for the designed molecule that defines, for each pair of atoms in the designed molecule, whether the pair of atoms are connected by a bond (728). The bond data for a designed molecule can further define one or more respective properties of each bond in the designed molecule, e.g., the type of the bond, e.g., single, double, or triple covalent bond, or ionic bond, or coordinate covalent bond, and so forth. In some implementations, the generative model can output the bond data for the designed molecule as part of generating the designed molecule. In some implementations, the system can generate the bond data for the designed molecule using a bond prediction machine learning model.

An example process for generating bond data for a designed molecule using a bond prediction machine learning model that processes atom embeddings generated for the atoms in the ligand by a generative model implemented as a generative diffusion model is described with reference to FIG. 8.

The system filters the set of designed molecules to remove any designed molecule that fails to satisfy each acceptance criterion in a set of one or more acceptance criteria (730). The set of acceptance criteria can include any appropriate acceptance criteria. A few examples of possible acceptance criteria are described next.

In one example, an acceptance criterion for a designed molecule can be that the designed molecule does not contain any structures that are designated as being physically impossible or highly unstable, e.g., a square planar carbon structure.

In another example, an acceptance criterion for a designed molecule can be that each atom in the designed molecule is bonded to at least one other atom in the designed molecule.

In another example, an acceptance criterion for a designed molecule can be that the value of a molecular property of the designed molecule satisfies one or more property-specific thresholds. The molecular property of the designed molecule can be, e.g., any of the global molecule properties or atom-specific molecule properties described earlier. The property-specific threshold for a molecular property can include, e.g., a lower bound on the value of the property, or an upper bound on the value of the property, or both.

The system can set the one or more property-specific thresholds for a molecule property in any appropriate way. For instance, the system can set a property-specific threshold for a molecular property based on a user input received from a user of the system by way of a user interface or an API made available by the system. As another example, the system can set a property-specific threshold for a molecular property based on a target value specified for the molecular property in the molecule design criteria processed by the system. For instance, if the molecule design criteria specify a target value for a molecular property, the system can set property-specific thresholds for the molecular property that include a lower bound and an upper bound on the value of the molecular property, where the lower and upper bound jointly define a range of values centered on the target value for the molecular property. Thus, the system can include acceptance criteria requiring that a designed molecule, in order to avoid being filtered from the set of possible designed molecules, must at least approximately satisfy some or all of the molecule design criteria.

As another example, an acceptance criterion for a designed molecule can be that, when the set of designed molecules are ranked based on the value of a particular molecular property, the designed molecule is included in a predefined number (e.g., 10, or 100, or 1000) or predefined percentage (e.g., 1%, or 5%, or 10%) of highest (or lowest) ranked designed molecules. For instance, the molecular property can be binding affinity for the target molecule, and the acceptance criterion can require that a designed molecule, in order to avoid being filtered from the set of possible designed molecules, must be among a predefined number or percentage of top-ranked designed molecules in the set of designed molecules when ranked based on binding affinity for the target molecule.

In order to evaluate whether a designed molecule satisfies an acceptance criterion (as described above), the system can computationally generate a value of a molecular property for the designed molecule. Certain molecular properties, such as molecular weight, number of rotatable bonds, number of rings, and so forth, may be directly and unambiguously derivable from the chemical structure of the designed molecule. However, in order to obtain the values of other molecular properties such as binding affinity, toxicity, absorption, and so forth, the system can process data characterizing the designed molecule (and, optionally, the target molecule) using a property prediction model that is configured to generate a predicted value of the molecular property.

The system can implement a property prediction model for a molecular property, e.g., as a machine learning model, e.g., a neural network, or a random forest, or a support vector machine, and so forth, having any appropriate machine learning model architecture. The system can train the property prediction model on a set of training examples and using a machine learning training technique.

Each training example for the property prediction machine learning model can correspond to a molecule and can include: (i) a training input characterizing the molecule, and (ii) an actual value of the molecular property. For each training example, the system can train the property prediction model to reduce a discrepancy between: (i) the actual value of the molecular property specified by the training example, and (ii) a predicted value of the molecular property generated by processing the training input specified by the training example using the property prediction model. The discrepancy between an actual value and a predicted value of a molecular property can be measured, e.g., as an absolute error, or a squared error, or in any other appropriate way. The machine learning training technique can be any appropriate technique appropriate for training the type of machine learning model used to implement the property prediction model. For instance, for a property prediction model implemented as a neural network, the design system can train the property prediction model using a stochastic gradient descent training technique.

As an example, when the system includes a property prediction neural network (e.g., the property prediction neural network 400 of FIG. 4 that can be jointly trained with the embedding neural network), the system can process data characterizing the designed molecule (and, optionally, the target molecule) using the property prediction neural network to generate a predicted value of the molecular property.

Filtering the set of designed molecules to remove any designed molecules that do not satisfy the acceptance criteria can have the effect of reducing the number of designed molecules in the set of designed molecules, e.g., by 50%, or 90%, or 99%.

After screening the set of designed molecules, the system provides the set of designed molecules as an output (732), e.g., by storing data defining the set of designed molecules in a memory, or by transmitting data defining the set of designed molecules over a data communication network, or by providing data defining the set of designed molecules directly to a system that performs downstream processing based on the designed molecules.

Optionally, one or more of the remaining designed molecules, i.e., that satisfy the acceptance criteria and remain in the set of designed molecules after the filtering, can be selected for further computational or physical validation.

Computational validation of a designed molecule can include, e.g., performing computational simulations such as quantum mechanics simulations (e.g., electronic structure calculations or molecular orbitals analysis), or molecular mechanics simulations (e.g., molecular dynamics simulations or Monte Carlo simulations), or docking simulations (e.g., protein-ligand docking simulations), and so forth. Computational simulations of a designed molecule can generate additional data characterizing the behavior, interactions, and properties of the designed molecule. In some cases, performing a computational simulation of a designed molecule can be computationally intensive, in particular, can require significant computational resources such as memory and computing power. Filtering the set of designed molecules and performing computational validation of only the designed molecules remaining after the filtering can thereby significantly reduce consumption of computational resources, e.g. as compared to performing computational validation of the entire set of designed molecules generated by the system.

Physical validation of a designed molecule can include, e.g., physically synthesizing the designed molecule and (in some cases) experimentally measuring one or more characteristics of the designed molecule, e.g., by measuring the binding affinity of the designed molecule for the target molecule, or by administering a drug including the designed molecule to a subject (e.g., a cell, or a collection of cells, or an animal, or a person) to assess the absorption, or distribution, or metabolism, or excretion, or toxicity of the drug that includes the designed molecule. Performing physical validation of a designed molecule can require significant resources, e.g., laboratory resources, chemical resources, personnel resources, and so forth. Filtering the set of designed molecules and performing physical validation of only the designed molecules remaining after the filtering can thereby significantly reduce consumption of resources, e.g., as compared to performing physical validation of the entire set of designed molecules generated by the system.

FIG. 7C illustrates generating a joint molecule embedding 106 representing a target molecule and molecule design criteria using an embedding neural network 108.

As described above, the embedding neural network 108 can process molecule data using to generate the joint molecule embedding 106. In particular, the embedding neural network 108 can process: (i) target molecule data 702 characterizing a target molecule, and optionally, (ii) molecule design criteria 704 specifying target molecule properties and/or scaffolding data, to generate the joint molecule embedding 106 representing the target molecule and (optionally) the molecule design criteria 704.

The embedding neural network 108 can include a molecule embedding neural network 742 and a design embedding neural network 744, which are each described in more detail next (and throughout this specification).

The molecule embedding neural network 742 is configured to process the target molecule data 702 characterizing the target molecule to generate a molecule embedding 746 of the protein. The molecule embedding 746 can include, e.g., a respective amino acid embedding of each amino acid in an amino acid sequence of the target molecule, a respective nucleotide embedding of each nucleotide in an nucleotide sequence of the target molecule, a respective atom embedding of each of one or more atoms in the target molecule, and so on.

In cases where the molecule design criteria 704 include target molecule scaffolding data, the molecule embedding neural network 742 can process both the target molecule data 702 and the target molecule scaffolding data.

The molecule embedding neural network 742 can have any appropriate neural network architecture that enables the molecule embedding neural network 742 to perform its described functions. In particular, the molecule embedding neural network 742 can include any appropriate types of neural network layers (e.g., fully connected layers, convolutional layers, attention layers, etc.) in any appropriate number (e.g., 5 layers, 10 layers, or 20 layers) and connected in any appropriate configuration (e.g., as a directed graph of layers).

Particular examples of possible architectures of the molecule embedding neural network 742 include the “Pairformer” neural network described in Abramson et al., “Accurate structure prediction of biomolecular interactions with AlphaFold 3,” Nature, Vol 630, 8 May 2024, and the “Evoformer” neural network described in Jumper et al., “Highly accurate protein structure prediction with AlphaFold,” Nature, Vol 596, 26 Aug. 2021.

The design embedding neural network 744 is configured to process the target molecule properties and/or designed molecule scaffolding data specified by the molecule design criteria 704 to generate a design embedding 748. If the input to the embedding neural network 108 does not include any target molecule properties or designed molecule scaffolding data, then the molecule prediction system 100 can bypass the operations of the design embedding neural network 744 and initialize the design embedding 308 as a default embedding.

The design embedding 748 can include a respective component embedding representing each molecule component (e.g., atom, group of atoms, amino acid, nucleic acid, etc.) in a set of possible molecule components that are eligible for inclusion in the designed molecule. The number of atoms that are included in the designed molecule to be generated by the design system may be unknown. Therefore, the number of component embeddings in the design embedding 748 can be defined by the molecule design criteria 704 or, if no molecule design criteria 704 are provided to the design system, can be set to a default value, e.g., 10, 50, 100, or 200 component embeddings. Each component embedding in the design embedding thus represents a molecule component (e.g., an atom, a group of atoms, an amino acid, a nucleic acid, etc.) that may (or may not) be selected for inclusion in the designed molecule generated by the generative model when conditioned on the joint molecule embedding 106, as will be described in more detail below. The number of component embeddings in the design embedding 748 can thus define a maximum number of molecule components that can be included in a designed molecule generated by the design system. The molecule components in the set of possible molecule components that are eligible for inclusion in the designed molecule may be referred to for convenience as “designed molecule components”.

If the input to the embedding neural network 108 does not include any target molecule properties or designed molecule scaffolding data, then the design system can initialize the design embedding 748 as a default embedding. In particular, the default design embedding 748 can include a respective component embedding representing each designed molecule component, where each component embedding is defined as a default embedding, e.g., an embedding where each value in the embedding is set to a predefined value, e.g., a value of zero or one.

The design embedding neural network 744 can have any appropriate neural network architecture that enables the design embedding neural network 744 to perform its described functions. In particular, the design embedding neural network 744 can include any appropriate types of neural network layers (e.g., fully connected layers, convolutional layers, attention layers, etc.) in any appropriate number (e.g., 5 layers, 10 layers, or 20 layers) and connected in any appropriate configuration (e.g., as a directed graph of layers).

An example of processing molecule design criteria 704 using a design embedding neural network 744 to generate a design embedding 748 is described in more detail with reference to FIG. 7D.

The fusion neural network 310 is configured to process the target molecule embedding 746 and the design embedding 748 to generate the joint molecule embedding 106 that jointly represents the target molecule data 702 and any molecule design criteria 704. The fusion neural network 126 can have any appropriate neural network architecture that enables the fusion neural network 126 to perform its described functions. In particular, the fusion neural network 126 can include any appropriate types of neural network layers (e.g., fully connected layers, convolutional layers, attention layers, etc.) in any appropriate number (e.g., 5 layers, 10 layers, or 20 layers) and connected in any appropriate configuration (e.g., as a directed graph of layers).

An example of processing a target molecule embedding 746 and a design embedding 748 using a fusion neural network 126 to generate a joint molecule embedding 106 is described in more detail with reference to FIG. 4A.

FIG. 7D is a flow diagram of an example process 750 for processing molecule design criteria using a design embedding neural network (e.g., that is included in the embedding neural network described with reference to FIG. 7C) to generate a design embedding. For convenience, the process 750 will be described as being performed by a system of one or more computers located in one or more locations. For example, a molecule prediction system, e.g., the molecule prediction system 100 of FIG. 1, appropriately programmed in accordance with this specification, can perform the process 750.

The system receives, by an input layer of the design embedding neural network, molecule design criteria specifying one or more target (desired) properties of the designed molecule and/or designed molecule scaffolding data (752). The molecule design criteria can specify global properties of the designed molecule (i.e., that characterize the designed molecule as a whole rather than being specific to a single atom) or atom-specific properties of the designed molecule (i.e., that relate to specific atoms in the designed molecule rather than the entire designed molecule) or both. The designed molecule scaffolding data can specify a respective 3D spatial position for each of one or more atoms of the designed molecule.

The system processes the molecule design criteria, by an embedding block of the design embedding neural network, to generate a collection of initial component embeddings that includes a respective initial component embedding for each molecule component (e.g., atom, group of atoms, amino acid, nucleic acid, etc.) of the designed molecule (754). If the molecule design criteria specify a maximum number of molecule components that can be selected for inclusion in the designed molecule, then the system can generate a number of initial component embeddings equal to the maximum number of molecule components that can be selected for inclusion in the designed molecule. If the molecule design criteria do not specify a maximum number of molecule components that can be selected for inclusion in the designed molecule, then the system can generate a default (e.g., predefined) number of initial component embeddings.

The system can include data representing the global properties of the designed molecule in all of the initial component embeddings. For each designed molecule component, the system can include: (i) any component-specific properties of the molecule component, and (ii) any designed molecule scaffolding data specifying a 3D spatial position of the molecule component, in the initial component embedding of the molecule component. Thus, for each designed molecule component, the system can generate the initial component embedding for the molecule component based on “component property data” that includes: (i) any global properties of the designed molecule, (ii) any component-specific properties of the molecule component, and (iii) any designed molecule scaffolding data for the molecule component.

To generate the initial component embedding for a molecule component, the system can generate an array (e.g., vector) of numerical values having a predefined dimensionality, where each value in the array is assigned to represent a respective type of component property data, e.g., global designed molecule property data, component-specific property data, and designed molecule scaffolding data. For instance, the array can include one or more values assigned to represent a target binding affinity of the ligand for the protein, and one or more values assigned to represent a type of the molecule component (e.g., an elemental type, an atomic composition, an amino acid identity, a nucleic acid identity, and so on), and one or more values assigned to represent designed molecule scaffold data for the molecule component, and so forth. The system can populate the array with component property data for the molecule component that includes: (i) any global properties of the designed molecule, (ii) any component-specific properties of the molecule component, and (iii) any designed molecule scaffolding data for the molecule component. Any values of the array that are not populated using the component property data are masked, e.g., are set to a default value (e.g., negative one).

In some implementations, the array of component property data for a molecule component directly defines the initial component embedding of the molecule component. In other implementations, the system processes the array of component property data for a molecule component using one or more neural network layers (e.g., fully connected layers) of the embedding block to generate the initial component embedding for the molecule component.

For one or more of the designed molecule components, the array of component property data for the molecule component may be partially or fully masked, e.g., if the molecule design criteria do not specify any global properties of the designed molecule, any component-specific properties of the molecule component, or any designed molecule scaffolding data for the molecule component.

The system processes the collection of initial component embeddings, by an update block of the design embedding neural network, to generate a collection of final component embeddings that includes a respective final component embedding for each molecule component of the designed molecule (756). In some implementations, the update block includes a sequence of one or more self-attention neural network layers that are each configured to receive a collection of atom embeddings, apply one or self-attention operations (e.g., query-key-value (QKV) self-attention operations) to the collection of input component embeddings to update each of the component embeddings, and then output the collection of updated component embeddings. The first self-attention layer in the sequence of self-attention layers can receive as input the collection of initial component embeddings generated by the embedding block, and each subsequent self-attention layer can receive as input the collection of component embeddings output by the preceding self-attention layer. The final self-attention layer in the sequence of self-attention layers can output collection of final component embeddings.

The system provides the collection of final component embeddings, by an output layer of the design embedding neural network, as the design embedding representing the molecule design criteria (758).

FIG. 7E provides an illustration of a collection of initial component embeddings generated by an embedding block of a design embedding neural network, as described at step 754 of FIG. 7D. Each initial component embedding represents a respective molecule component of the designed molecule. Any global designed molecule properties specified by the molecule design criteria are broadcast across all of the initial component embeddings. Any component-specific properties or designed molecule scaffolding data that are specific to a particular molecule component are separately represented in the initial component embedding for that molecule component.

FIG. 7F is a flow diagram of an example process 760 for generating data defining a designed molecule based on the denoised molecule component state data for each molecule component of the designed molecule. For convenience, the process 760 will be described as being performed by a system of one or more computers located in one or more locations. For example, a molecule prediction system, e.g., the molecule prediction system 100 of FIG. 1, appropriately programmed in accordance with this specification, can perform the process 760.

The system receives respective denoised component state data for each molecule component of the designed molecule (762). The denoised component state data for a molecule component refers to a current estimate of the denoised component state data generated for the designed molecule component at the final denoising time step in the sequence of denoising time steps, as described below with reference to FIG. 10A.

The system selects a subset of the set of designed molecule components for inclusion in the designed molecule (764). In particular, as described above, each designed molecule component in the set of designed molecule components represents a molecule component that is eligible for inclusion in the designed molecule. The system is not aware, prior to generating the denoised component state data for the designed molecule components, which designed molecule components will be selected for inclusion in the designed molecule and which designed molecule components will be discarded. The number of designed molecule components in the set of designed molecule components thus represents the maximum number of molecule components that can be included in the designed molecule.

For each designed molecule component, the system can determine whether to select the designed molecule component for inclusion in the designed molecule based on the 3D spatial position of the designed molecule component as defined by the denoised component state data for the designed molecule component. For instance, the system can determine that a designed molecule component should be selected for inclusion in the designed molecule only if the 3D spatial position of the designed molecule component is at least a threshold distance from a predefined 3D spatial position referred to as the “throw-away” position. In this example, the system may have trained the generative diffusion model to move designed molecule components that are not included in the designed molecule to the throw-away position. Training the generative diffusion model to move unneeded molecule components to the throw-away position is described in more detail below with reference to FIG. 10E.

The system filters the set of designed molecule components to remove any designed molecule components that are not selected for inclusion in the designed molecule (766). Filtering the set of designed molecule components to remove designed molecule components that are not selected for inclusion in the designed molecule may reduce the number of molecule components in the set of the designed molecule components by any appropriate amount, e.g., by 10%, or 50%, or 90%. After the filtering, a molecule component is included in the set of designed molecule components if and only if it represents an molecule component that is selected for inclusion in the designed molecule. In some cases, the system may select all the molecule components in the original set of designed molecule components for inclusion in the designed molecule and thus no molecule components are filtered from the set of designed molecule components.

For each molecule component in the set of designed molecule components, the system selects a respective value for each categorical feature that is represented in the denoised component state data for the molecule component (768). A categorical feature can refer to a feature having a value that is selected from a finite set of possible values. Examples of categorical features can include, e.g., atomic hybridization state, atomic element type, atomic composition, amino acid identity, nucleic acid identity, and so on. The denoised component state data for a molecule component can represent a categorical feature by a distribution over the set of possible values of the categorical feature, where the distribution assigns a respective score to each possible value of the categorical feature.

The system can select a value for a categorical feature of a designed molecule component based on the distribution over possible values of the feature that is included in the denoised component state data for the molecule component in any of a variety of possible ways. For instance, the system can select the value that is assigned the highest score by the distribution over the set of possible values of the feature as the value of the categorical feature. As another example, the system can sample the value of the categorical feature from a probability distribution defined by the distribution over the set of possible values of the feature.

For each molecule component in the set of designed molecule component, the system selects a respective value for each continuous feature that is represented in the denoised component state data for the molecule component (770). A continuous feature can refer to a feature having a value that is selected from a continuous set of possible values. Examples of continuous features can include, e.g., 3D spatial position, atomic partial charge, and so forth. The denoised component state data for a molecule component can directly represent a continuous feature in one or more assigned dimensions of the denoised atom state data, and the system can select the value for the continuous feature by extracting the corresponding dimensions representing the value of the continuous feature from the denoised atom state data for the atom.

The system provides data defining the designed molecule (772). The data defining the designed molecule includes data identifying each molecule component that is included in the designed molecule, and a set of features associated with each molecule component that is included in the designed molecule. The set of features associated with a molecule component that is included in the designed molecule can include continuous features, e.g., the 3D spatial position of the component, the partial charge of the component, and categorical features, e.g., the hybridization state of the component, an element type of the component, an amino acid identity of the component, a nucleic acid identity of the component, and so on.

FIG. 8 is a flow diagram of an example process 800 for generating bond data for a molecule generated by a molecule design neural network. For convenience, the process 800 will be described as being performed by a system of one or more computers located in one or more locations. For example, a molecule prediction system, e.g., the molecule prediction system 100 of FIG. 1, appropriately programmed in accordance with this specification, can perform the process 800.

The system receives a respective atom embedding for each atom in the molecule (802).

In some implementations, the atom embeddings of the atoms of the molecule are generated, e.g., as an intermediate output of the generative model that generates the molecule. For instance, for a generative diffusion model implemented using a denoising neural network, as described with reference to FIG. 10A-FIG. 10D, the system can receive a respective atom embedding for each atom in the molecule that is generated by the denoising neural network during a final denoising time step in a sequence of denoising time steps performed by generative diffusion model. In particular, for each atom in the molecule, the system can receive a respective atom embedding for the atom that is generated as an output of an update block of the denoising neural network at the final denoising time step in the sequence of denoising time steps, as described at step 1026 of FIG. 10B. That is, the denoising neural network may have an architecture that comprises a sequence of update blocks (e.g. a U-Net, DiT, or U-ViT architecture), and the atom embeddings may be generated as the output from one of these update blocks.

In some implementations, for each atom in the molecule, the atom embedding of the atom comprises a collection of atom features characterizing the atom, e.g., including one or more of: the 3D spatial position of the atom (e.g., in a complex with a protein), the hybridization state of the atom, the partial charge of the atom, the element type of the atom, and so forth. The atom features characterizing the atoms in the ligand can generated as part of the output of the generative model that generates the molecule, e.g., as described in FIG. 10B.

The system processes the 1D sequence of atom embeddings of the atoms in the molecule to generate a 2D array of “pair” embeddings (804) (where each pair embedding represents a pair of atoms). The 2D array of embeddings can be represented by data having dimensionality NumAtoms×NumAtoms×d where NumAtoms is the number of atoms in the molecule and d is a positive integer value defining the number of channel dimensions in each embedding. The system can generate the 2D array of pair embeddings from the 1D sequence of atom embeddings in any of a variety of ways. For instance, the system can generate the 2D array of pair embeddings as a result of an element-wise outer product of the 1D sequence of atom embeddings with itself. As another example, the system can generate the 2D array by an appropriate 2D concatenation operation, e.g., where the embedding at each position (i, j) in the 2D array of pair embeddings is generated by concatenating: (i) the atom embedding at position i, and (ii) the atom embedding at position j, in the 1D sequence of atom embeddings (where indices i, j∈{1, . . . . N}, where N is the number of atoms in the molecule).

Optionally, the system receives data identifying a respective 3D spatial position of each atom in the molecule (806). The 3D spatial positions of the atoms in the molecule can be generated the generative model, as described throughout this specification.

Optionally, the system processes the 3D spatial positions of the atoms in the molecule to generate a 2D spatial distance array that, at position (i, j), represents a spatial distance between atom i and atom j in the molecule (808).

Optionally, the system updates the 2D array of pair embeddings based on the 2D spatial distance array (810). For instance, the system can channel-wise concatenate the 2D spatial distance array to the 2D array of pair embeddings, i.e., such that the 2D array of pair embeddings has dimensionality NumAtoms×NumAtoms×(d+1), where (as above) NumAtoms is the number of atoms in the molecule, and d is a positive integer value defining the number of channel dimensions in each pair embedding prior to the channel-wise concatenation.

The system processes the 2D array of pair embeddings using a bond prediction machine learning model to generate bond data that bond data that defines, for each pair of atoms in the molecule, whether the pair of atoms are connected by a bond (812). The bond data can further define one or more respective properties of each bond in the molecule, e.g., the type of the bond, e.g., single, double, or triple covalent bond, or ionic bond, or coordinate covalent bond, and so forth.

For instance, the bond prediction machine learning model can generate a model output that defines, for each pair of atoms in the molecule, a distribution over a set of possible bond categories. The set of possible bond categories includes a “no bond” category, indicating that the pair of the atoms are not connected by a bond, and a respective category for each of multiple types of bonds that can exist between the pair of atoms, e.g., single, double, or triple covalent bond, or ionic bond, or coordinate covalent bond, and so forth.

For each pair of atoms in the molecule, the bond prediction machine learning model can select a bond category for the pair of atoms based on the corresponding distribution over the set of possible bond categories. For instance, the system can select the bond category for the pair of atoms as the bond category having the highest score under the distribution over the set of bond categories. As another example, the system can sample the bond category for the pair of bonds from distribution over the set of bond categories.

The bond prediction machine learning model can be any appropriate type of machine learning model. For instance, the bond prediction machine learning model can be a neural network, or a random forest, or a support vector machine, and so forth. In a particular example, the bond prediction machine learning model can be implemented as a neural network having any appropriate neural network architecture, in particular, an architecture that includes any appropriate types of neural network layers (e.g., fully connected layers, attention layers, convolutional layers, and so forth) in any appropriate number (e.g., 5 layers, 10 layers, or 20 layers) and connected in any appropriate configuration (e.g., as a directed graph of layers).

The system can train the bond prediction machine learning model on a set of training examples using a machine learning training technique. Each training example corresponds to an example molecule and can include: (i) a training input to the bond prediction machine learning model, and (ii) target bond data for the example molecule. The training input to the bond prediction machine learning model can include respective atom embedding for each atom in the example molecule, and optionally, 3D spatial position data for each atom in the example molecule. For each training example, the system trains the bond prediction machine learning model to reduce a discrepancy between: (i) target bond data specified by the training example, and (ii) predicted bond data generated by the bond prediction machine learning model by processing the training input of the training example.

In some implementations, the bond prediction machine learning model is implemented as a neural network, the generative model is implemented as a generative diffusion model parameterized by a denoising neural network, and the bond prediction machine learning model receives atom embeddings that are generated as an intermediate output of the denoising neural network (as described above). In these implementations, the system can train the bond prediction machine learning model jointly with the embedding neural network and the denoising neural network.

More specifically, for each training example, the training input includes atom embeddings generated as an intermediate output of the denoising neural network. As part of training the bond prediction machine learning model on a training example, the system can determine gradients (e.g., by backpropagation) of an objective function (e.g., a cross-entropy objective function) that measures a discrepancy between: (i) the target bond data specified by the training example, and (ii) predicted bond data generated by the bond prediction machine learning model by processing the training input of the training example. In particular, the system can determine gradients of the objective function with respect to not only the set of parameters of the bond prediction machine learning model, but also with respect to parameters of the denoising neural network and the embedding neural network. The system can then adjust the parameters of the bond prediction machine learning model, the denoising neural network, and the embedding neural network using the gradients, e.g., in accordance with the update rule of an appropriate gradient descent optimization algorithm, e.g., RMSprop or Adam. That is, the system can backpropagate gradients of the objective function through the bond prediction neural network and into the denoising neural network and the embedding neural network.

FIG. 9 is a flow diagram of an example process for in-painting and/or out-painting a protein-ligand complex. For convenience, the process 900 will be described as being performed by a system of one or more computers located in one or more locations. For example, a molecule prediction system, e.g., the molecule prediction system 100 of FIG. 1, appropriately programmed in accordance with this specification, can perform the process 900.

The system receives respective atom properties for each atom in a complex comprising a input protein and an input ligand (902). Atom properties for an atom can define one or more of: a 3D spatial position of the atom (e.g., when the protein and the ligand are bound in a complex), a hybridization state of the atom, a partial charge of the atom, or an element type of the atom.

The system receives a request to perform in-painting of the complex, or out-painting of the complex, or both (904).

A request to in-paint the complex identifies, for each atom property of each atom in the complex, whether the atom property is a “static” property or a “variable” property. In-painting the complex refers to generating new ligands that have the static atom properties of the input ligand and that bind to proteins that have the static atom properties of the input protein.

In implementations where the system receives a request to perform in-painting, the system receives input data that, for each atom property of each atom in the complex, identifies the atom property as a static property or as a variable property. The input data identifies at least one atom property of at least one atom in the ligand as being a variable property. The system can receive the input that identifies the static and variable atom properties, e.g., from a user or from an upstream system, e.g., by way of a user interface (e.g., a graphical user interface) made available by the system, or by an API made available by the system. For instance, a graphical user interface can allow a user to dynamically select parts of the complex as variable or static, e.g., by interacting with a 3D representation of the complex presented to the user by way of the user interface.

A request to out-paint the complex defines a request to generate new ligands that expand on the input ligand, e.g., by including one or more new (additional) atoms relative to the input ligand.

In implementations where the system receives a request to perform in-painting, the system generates molecule design criteria that include the static atom properties for the atoms in the complex (906). The molecule design criteria can include, e.g., one or more of: ligand scaffolding data, atom-specific properties of atoms in the ligand, protein scaffolding data, or atom-specific properties of atoms in the protein.

The system processes an input including any molecule design criteria, e.g., using the embedding neural network and the generative model described throughout this specification, to generate data defining one or more ligands (908). In implementations where the system performs in-painting, the molecule design criteria are selected (as described above) such that the system attempts to generate ligands that have the static atom properties of the ligand atoms and that bind to proteins that have the static atom properties of the protein atoms. That is, the system attempts to generate ligands that in-paint the variable atom properties in a variety of possible ways. In some cases, the system generates ligands that each differ from the ligand received as an input at step 902, e.g., as a result of stochasticity in the operations of the generative model.

In implementations where the system performs out-painting of the ligand, the system can generate a design embedding of the molecule design criteria that includes a respective atom embedding for each atom in a set of atoms that includes: (i) all the atoms included in the input ligand, and (ii) one or more “out-painted” ligand atoms. The out-painted ligand atoms represent atoms that the generative model can select for inclusion in a new ligand as part of out-painting the input ligand.

FIG. 10A is a flow diagram of an example process 1000 for generating a predicted joint 3D structure of a molecule complex using a generative diffusion model that includes a denoising neural network. For convenience, the process 1000 will be described as being performed by a system of one or more computers located in one or more locations. For example, a molecule prediction system, e.g., the molecule prediction system 100 of FIG. 1, appropriately programmed in accordance with this specification, can perform the process 1000. A similar process can be used with models that are similar to a diffusion model, e.g. a consistency model.

The system generates respective initial atom state data for each atom in the molecule complex (1002).

The atom state data for an atom can include features that can be categorized as “continuous features” or “categorical features.”

A continuous feature refers to a feature that can take on values from a continuous range of possible values, e.g., in a continuous interval. The atom state data for an atom can include continuous features such as a feature defining a 3D spatial position of the atom (e.g., which can assume values from in the continuous space [symbol] ³), a feature defining a partial charge of the atom (e.g., which can assume values in the continuous interval [−1,+1]), and so forth.

A categorical feature can refer to a feature that can assume a value in a finite set of possible values. The atom state data for an atom can include categorical features such as a feature defining an elemental type of the atom (e.g., which can assume values from a discrete set of possible elemental types, e.g., carbon, oxygen, nitrogen, etc.), a feature defining a hybridization state of the atom (e.g., which can assume values from a discrete set of possible hybridization states, e.g., sp hybridization, or sp² hybridization, or sp³ hybridization, or sp³d hybridization, or sp²d² hybridization, etc.), and so forth.

Generally, for each atom, the atom state data for the atom specifies at least a 3D spatial position of the atom. Optionally, the atom state data for each atom can further specify one or more additional atom-specific properties of the atom, e.g., including one or more of: an elemental type of the atom, a hybridization state of the atom, a partial charge of the atom, and so forth.

The system can stochastically sample the initial atom state data for some or all of the atoms in the complex, i.e., for some or all of the atoms in molecule of the molecule complex. In particular, for some or all of the continuous features in the atom state data for an atom, the system can sample a value of the feature from a probability distribution over the space of possible values of the feature. The system can then include the sampled value of the feature in the initial atom state data for the atom.

For instance, the system can sample a 3D spatial position of an atom from a probability distribution over 3D space, and then include the sampled 3D spatial position in the initial atom state data for the atom. The probability distribution over 3D space can be, e.g., a standard Normal distribution over 3D space. The system can represent the sampled 3D spatial position in the initial atom state data for the atom, e.g., in Cartesian coordinates or in spherical coordinates or in any other appropriate coordinate system over 3D space.

As another example, the system can sample a value for a partial charge of an atom from a probability distribution (e.g., a uniform distribution) over a range of possible partial charges (e.g., the interval [−1,+1]). The system can then include the sampled value of the partial charge in the initial atom state data for the atom.

Further, for some or all of the categorical features in the atom state data for an atom, the system can sample a distribution over a set of possible values of the feature. The system can then include the distribution over possible values of the feature in the initial atom state data for the atom. The distribution over the set of possible values of the feature can define a respective score for each value in the set of possible values of the feature. The system can sample the score for each possible value of the feature from a probability distribution over a range of possible scores. Optionally, the system can normalize the distribution over the set of possible values of the feature prior to including the distribution in the initial atom state data, e.g., by processing the distribution using a soft-max function.

For instance, in some implementations, the system can generate a distribution over a set of possible atomic element types of an atom, e.g., by sampling a respective score for each possible atomic element type from a probability distribution over a range of possible scores, e.g., a uniform distribution over the range [0,1]. The system can then include the distribution over the set of possible atomic element types of the atom in the initial atom state data for the atom.

As another example, the system can generate a distribution over a set of possible atomic hybridization states of an atom, e.g., by sampling a respective score for each possible atomic hybridization state from a probability distribution over a range of possible scores, e.g., a uniform distribution over the range [0,1]. The system can then include the distribution over the set of possible atomic hybridization states of the atom in the initial atom state data for the atom.

In some cases, the system can receive scaffolding data (e.g., protein scaffolding data, ligand scaffolding data, or both) that specifies a respective target 3D spatial position of each of one or more atoms in the molecule complex. If the system receives scaffolding data specifying a target 3D spatial position of an atom in the complex, then the system can generate initial atom state data for that atom that includes the target 3D spatial position of the atom rather than stochastically sampled 3D spatial position data for the atom (as described above).

In some cases, the system can receive molecule design criteria that specify one or more atom-specific target properties (e.g., target hybridization state data, or target partial charge data) of ligand atoms in a ligand of the molecule complex. If the system receives molecule design criteria specifying an atom-specific target property of a ligand atom, then the system can generate initial atom state data for that atom that includes the atom-specific target property of the ligand atom rather than stochastically sampled atom property data (as described above).

In particular, if the system receives molecule design criteria specifying a particular value of a continuous feature of an atom (e.g., a 3D spatial position or a partial charge of the atom), then the system can include that feature value in the initial atom state data for the atom (e.g., instead of a stochastically sampled value for that feature).

If the system receives molecule design criteria specifying a particular value of a categorical feature of an atom (e.g., an element type of the atom or a hybridization state of the atom), then the system can generate a distribution over a set of possible values of the feature that assigns a first value (e.g., one) to the actual value of the feature and a second value (e.g., zero) to each other value of the feature. For instance, the system can generate a one-hot distribution over the set of possible values of the feature that uniquely identifies the actual value of the feature. The system can then include the generated distribution over possible values of the feature in the initial atom state data for the atom (e.g., instead of a stochastically sampled distribution over possible values of the feature).

For each atom in the complex, the initial atom state data for the atom thus defines a “noisy” representation of the state of the atom. The system performs steps 1004-1012, which are described next, over a sequence of iterations that may be referred to as “denoising time steps,” to progressively denoise the initial atom state data for the atoms as part of a predicted joint 3D structure for the molecule complex. More specifically, the system progressively denoises the initial atom state data for each atom in the complex to cause the noisy representation of the atom state to converge on a denoised representation that represents the atom state in the predicted 3D structure of the molecule complex.

The description of steps 1004-1010 which follows will reference a “current” denoising time step for convenience; the current denoising time step can be any denoising time step in the sequence of denoising time steps. The system can perform the steps 1004-1010 over any appropriate number of denoising time steps, e.g., 3 denoising time steps, 10 denoising time steps, or 100 denoising time steps. The number of denoising time steps can be a predetermined number of time steps.

The system generates a denoising output by processing respective current atom state data for the atoms in the complex using a denoising neural network that is conditioned on the joint molecule embedding (1004). If the current denoising time step is the first denoising time step, then the current atom state data for the atoms in the complex may be the initial atom state data, e.g., as generated at step 1002. If the current denoising time step is after the first denoising time step, then the current atom state data for the atoms in the complex may be generated at the preceding denoising time step, e.g., as in step 1012 of the process 1000, which will be described in more detail below.

The denoising output can be any appropriate data that enables estimation of the denoised (ground truth) atom state data of each atom in the complex. For instance, the denoising output can define, for each atom in the complex, a predicted error in the atom state data of the atom at the current denoising time step. As another example, the denoising output can directly define, for each atom in the complex, predicted atom state data for the atom. As another example, the denoising output can define, for each atom in the complex, both: (i) a predicted error in the atom state data for the atom at the current denoising time step, and (ii) predicted atom state data for the atom. As another example, the denoising output can define, for each atom in the complex, a prediction for an array of values that is a linear combination of: (i) actual atom state data for the atom, and (ii) an error between the atom state data for the atom at the current denoising time step and the actual atom state data for the atom, e.g., as implemented by the v-parametrization described in: Tim Salimans, Jonathan Ho, “Progressive distillation for fast sampling of diffusion models,” ICLR 2022, arXiv: 2202.00512v2. An example process for generating a denoising output using the denoising neural network is described in more detail with reference to FIG. 10B.

Optionally, in addition to generating the denoising output, the system can generate gradients, with respect to the current atom state data for some or all of the atoms in the complex, of each of one or more conditioning objective functions (1006). Each conditioning objective function is associated with a respective property of the molecule complex and measures a discrepancy between: (i) a predicted value of the property for the molecule complex as defined by the current atom state data, and (ii) a target (desired) value of the property. The system can generate the predicted value of the property, e.g., by processing the current atom state data for some or all of the atoms in the complex using a respective property prediction neural network, as will be described in more detail with reference to FIG. 10D. The system can receive the target (desired) value of the property, e.g., as an input from a user of the system or from an upstream system, e.g., by way of a user interface or an API.

As an example, when the molecule complex is a protein-ligand complex that includes a protein and a ligand, one or more conditioning objective functions can be associated with ligand properties of the ligand.

The gradients of a conditioning objective function associated with a property can be used to update the current atom state data to reduce a discrepancy between the predicted value of the property for the molecule complex defined by the current atom state data and the target value of the property. Thus, the system can use the gradients (in addition to the denoising output of the denoising neural network) to steer (influence) the process of denoising the current atom state data to increase the likelihood that the resulting molecule complex will assume the target value for the property. An example of using gradients of a conditioning objective function to update the current atom state data is described in more detail below with reference to step 1008 of the process 1000.

An example process for determining gradients of a conditioning objective function with respect to the current atom state data for some or all of the atoms in the complex is described in detail with reference to FIG. 10D.

The system generates a current estimate of the denoised atom state data for each atom in the complex using the denoising output generated by the denoising neural network, and optionally, respective gradients of each conditioning objective function (1008).

In implementations where the system generates gradients of a conditioning objective function, the system can combine the gradients of the conditioning objective function with the denoising output of the denoising neural network prior to using the denoising output to generate the current estimate of the denoised atom state data. The system can combine the gradients of the conditioning objective function with the denoising output of the denoising neural network, e.g., by scaling the gradients of the conditioning objective function by a scaling constant. The scaling constant can determine a level of guidance of the generation by the conditioning objective function, and can depend on the current time step. The gradients of the conditioning objective function can be added to the denoising output of the denoising neural network. That is, a technique similar to classifier guidance of a diffusion model can be used.

More specifically, the denoising output of the denoising neural network can include a respective value, referred to for convenience as a denoising value, for each component of the atom state data for each atom in the complex. The gradients of the conditioning objective function include a respective gradient value for some or all of the components of the atom state data for some or all of the atoms in the complex. The system can scale the gradients of the conditioning objective function by the scaling constant (as described above), and then combine (e.g., by addition) each gradient value with the corresponding denoising value that is associated with the same component of the atom state data as the gradient value.

After (optionally) combining the gradients of each conditioning objective function with the denoising output, the system can generate the current estimate of the denoised atom state data for each atom in the complex in any appropriate way, depending on the form of the denoising output. A few example techniques for generating the current estimate of the denoised atom state data for each atom in the complex using the denoising output are described next.

In one example, the denoising output defines, for each atom, a respective prediction for the atom state data of the atom. In this example, the respective predicted atom state data for each atom defines the current estimate of the denoised atom state data for the atom.

In another example, the denoising output defines, for each atom, a predicted error in the atom state data for the atom at the current time step. In this example, the system can generate the current estimate for the denoised atom state data for each atom as a linear combination of: (i) the current atom state data for the atom, and (ii) the predicted error in the atom state data for the atom. Each term in the linear combination can be scaled by a respective constant value that is dependent on the time step. For instance, the system can generate the current estimate for the denoised atom state data xₜ₋₁ for an atom in the complex as:

$${x}_{t - 1} = {α}_{t}^{- 0.5} ( {x}_{t} - ( 1 - {α}_{t} ) {( 1 - {\overline{α}}_{t} )}^{- 0.5} {ϵ}_{θ} ( {x}_{t} , t ) ) \qquad ( 1 )$$

where t indexes the current time step, αₜ, α̅ₜ, and σₜ are constants specific to time step t, and ϵθ(xₜ, t) is the predicted error in the atom state data of the atom (e.g., as generated by the denoising neural network at the time step). (In the notation of equation (1), the time steps decrement, such that time step t−1 is the “next” time step after time step t). The constants in equation (1) (αₜ and α̅ₜ) can be selected in accordance with a predefined noise schedule. For example αₜ=1−βₜ, α̅ₜ can be the cumulative product of values of at from an initial time step up to current time step t, and βₜ (which represents a noise schedule of the forward process) can decrease, e.g. from 1 to 0 (e.g. linearly or with a cosine or any other dependence), as the time steps decrement In the example of equation (1) the sampling is deterministic and the noise variance,

$${σ}_{t}^{2} = 0$$

probabilistic setting it can depend on 1−αₜ and a mean μₜ₋₁, rather than xₜ₋₁, can be determined).

In another example, the denoising output defines, for each atom, both: (i) predicted atom state data for the atom, and (ii) a predicted error in the atom state data for the atom at the current time step. In this example, the system can generate the current estimate for the denoised atom state data for the atom as a combination (e.g., an average) of: (i) the predicted atom state data for the atom as specified by the denoising output, and (ii) predicted atom state data for the atom that is derived from the predicted error in the atom state data for the atom at the current time step, e.g., using equation (1).

In another example, the denoising output is expressed using a v-parametrization, and the system generates a respective current estimate for the denoised atom state data for each atom using the techniques described in Tim Salimans, Jonathan Ho, “Progressive distillation for fast sampling of diffusion models,” ICLR 2022, arXiv: 2202.00512v2.

Optionally, the system can generate a respective confidence measure for the current estimate of the respective denoised atom state data for each atom in the complex. For instance, as part of generating the denoising output, the denoising neural network can generate a respective atom embedding for each atom in the complex, e.g., as the output of the update block of the denoising neural network, as described with reference to step 1026 of FIG. 10B. The system can process each atom embedding using one or more neural network layers (e.g., a combination of one or more of: fully connected layers, or attention layers, or pooling layers) to generate a respective confidence estimate for the current estimate of the denoised atom state data of the atom(s) represented by the atom embedding. The confidence measure for the current estimate of the denoised atom state data for an atom can characterize a predicted error in the current estimate of the atom state data for the atom.

Optionally, the system can generate a respective confidence measure for the current estimates of the 3D spatial positions of pairs of atoms in the complex. The current estimate for the 3D spatial position of an atom in the complex refers to the 3D spatial position of the atom that is defined by the current estimate of the denoised atom state data for the atom. For instance, for a first atom in the complex and a second atom in the complex, the system can process an atom embedding representing the first atom and an atom embedding representing the second atom (e.g., as generated by the update block of the denoising neural network) using one or more neural network layers (e.g., a combination of one or more of: fully connected layers, or attention layers, or pooling layers) to generate a confidence estimate for the current estimates of the 3D spatial positions of the first atom and the second atom. The confidence measure can characterize, e.g., a predicted error in the relative 3D displacement of the first atom and the second atom.

Optionally, the system can generate a confidence measure for a respective structure of each of one or more molecules within the complex (e.g., proteins, ligands, etc.) as defined by the initial estimates of the 3D spatial positions of the atoms in the molecule, e.g., by combining (e.g., summing or averaging) the confidence measures for the individual atoms included in the molecule.

Optionally, the system can generate a confidence measure for a structure of an interface between molecules of the complex (e.g., between a protein and a ligand of the molecule complex), e.g., by combining (e.g., summing or averaging) the confidence measures of pairs of atoms included in the interface. A pair of atoms can be referred to as being included in the interface, e.g., if the pair includes: (i) a first atom included in a first molecule of the complex (e.g., a ligand), and (ii) a second atom included in a second molecule of the complex (e.g., a protein), where the relative displacement between the 3D spatial positions of the atoms is less than a threshold, e.g., 2 Angstroms, or 3 Angstroms, or 8 Angstroms. An example of generating a confidence measure for a pair of atoms is described above.

If the current denoising time step is not the final denoising time step, the system generates respective atom state data for each atom in the complex for the next denoising time step based on the current estimates of the denoised atom state data for the atoms (as generated at step 1008) using an appropriate diffusion sampling technique (1012). A few examples of possible diffusion sampling techniques are described next.

In one example, the system can generate the atom state data for each atom in the complex at the next denoising time step by combining random noise with the current estimate of the denoised atom state data for the atom. For instance, for each atom, the system can add respective random noise to the current estimate of the denoised atom state data for the atom. The random noise can be sampled from an appropriate probability distribution. The probability distribution can vary based on the time step, e.g., such that the variance of the noise combined with the updated atom state data for the atoms decreases over the sequence of time steps. An example is the denoising diffusion probabilistic model (DDPM), e.g., as described in Ho et al. arXiv: 2006:11239.

As another example, the system can generate the atom state data for each atom in the complex at the next denoising time step using a deterministic diffusion sampling technique, i.e., that does not rely on random noise. An example of a deterministic diffusion sampling technique is the denoising diffusion implicit model (DDIM), e.g., as described in: Jiaming Song, Chenlin Meng, Stefano Ermon, “Denoising diffusion implicit models,” ICLR 2021, arXiv: 2010.02502v4.

Optionally, when the system receives molecule design criteria, the system can refrain from applying the diffusion sampling technique to any component of the atom state data for an atom that was initialized at step 1002 to represent target values specified by the molecule design criteria. More specifically, for any component of the atom state data for an atom that was initialized at step 1002 to represent a target value specified by the molecule design criteria, the system can set the value of the component in the atom state data for the atom for the next denoising time step to be the same as the value of the component in the current estimate of the denoised atom state data.

If the current denoising time step is the final denoising time step in the sequence of denoising time steps, the system generates data characterizing the predicted joint 3D structure of the molecule complex based on the denoised atom state data (1014). The denoised atom state data for an atom refers to the current estimate of the denoised atom state data generated for the atom at the final denoising time step in the sequence of denoising time steps.

The system can output data characterizing some or all of the predicted joint 3D structure of the molecule complex.

For example, the system can output data specifying a 3D structure of the entire molecule complex, i.e., as defined by the 3D spatial position data included in the denoised atom state data for each atom in the molecule complex.

As another example, the system can output data specifying a 3D structure of each of one or more molecules of the molecule complex, i.e., as defined by the 3D spatial position data included in the denoised atom state data for each atom in the molecule.

As another example, the system can output data identifying a binding pocket of the molecule complex where a first molecule of the complex (e.g., a ligand) to a second molecule of the complex (e.g., a protein). In particular, when the molecule complex is a protein-ligand complex that includes a protein and a ligand, the system can identify any amino acid residue in the protein which includes an atom that is within a threshold distance (e.g., 2 Angstroms) of at least one atom in the ligand as being included in the binding pocket. The data identifying the binding pocket can include data identifying each of the amino acid residues in the protein that are included in the binding pocket.

Optionally, the system can perform the process 1000 multiple times to generate data characterizing multiple predicted 3D structures for the molecule complex. Each execution of the steps of the process 1000 can result in the generation of a different predicted 3D structure, e.g., as a result of stochasticity in the random sampling performed to generate the initial atom state data for the atoms (at step 1002), and in some cases, as a result of stochasticity in the diffusion sampler (at step 1012).

FIG. 10B is a flow diagram of an example process 1020 for generating a denoising output using a denoising neural network conditioned on a joint molecule embedding. For convenience, the process 1020 will be described as being performed by a system of one or more computers located in one or more locations. For example, a molecule prediction system, e.g., the molecule prediction system 100 of FIG. 1, appropriately programmed in accordance with this specification, can perform the process 1020.

The system receives: (i) data defining a respective current 3D spatial position of each atom in the complex, and (ii) the joint molecule embedding (1022). The joint molecule embedding can include component-component embeddings for pairs of molecule components (e.g., amino acids, atoms, etc.) within the molecule complex, as described in more detail above with reference to FIG. 2B. For example, when the joint molecule embedding represents a protein and a ligand, the joint molecule embedding can include: (i) atom—atom embeddings, (ii) amino acid—amino acid embeddings, and (iii) amino acid—atom embeddings. The system can optionally receive additional inputs, e.g., an input defining a current time step in a diffusion process being implemented by a generative diffusion model, as described above with reference to FIG. 10A.

The system generates a respective component embedding for each molecule component (e.g., atom, amino acid, etc.) in the complex, using an encoder block of the generative neural network, based at least in part on the current 3D spatial position of the atom (1024). Optionally, the system can generate the atom embeddings for the atoms based at least in part on the joint molecule embedding (and, optionally, the current time step in the diffusion process), e.g., in addition to the current 3D spatial positions of the atoms. For instance, for each atom, the system can generate the atom embedding of the atom based on both: (i) the current 3D spatial position of the atom, and (ii) a respective conditioning embedding selected from the collection of embeddings included in the joint molecule embedding. For an atom included in an amino acid in the complex, the conditioning embedding for the atom can be the amino acid—amino acid embedding (i.e., from the joint molecule embedding) of the amino acid that includes the atom (i.e., the amino acid—amino acid embedding corresponding to a pair of amino acids that includes two copies of the amino acid). For an atom included in a ligand in the complex, the conditioning embedding for the atom can be the atom—atom embedding (i.e., from the joint molecule embedding) of the atom (i.e., the atom—atom embedding corresponding to a pair of atoms that includes two copies of the atom).

The system can generate the atom embedding for an atom based on the current 3D spatial position of the atom (and, optionally, a conditioning embedding for the atom) in any of a variety of possible ways. For instance, the system can generate the atom embedding for an atom by processing the 3D spatial position of the atom using an encoder block of the denoising neural network. As another example, the system can generate the atom embedding for an atom by processing both: (i) the 3D spatial position of the atom, and (ii) the conditioning embedding for the atom, using an encoder subnetwork of the denoising neural network. As another example, the system can generate the atom embedding for an atom by concatenating: (i) an output generated by an encoder subnetwork of the denoising neural network by processing the 3D spatial position of the atom, and (ii) the conditioning embedding for the atom.

Optionally, for each atom, the system can further generate the respective atom embedding of the atom based on data, e.g., a set of binary flags, indicating which (if any) components of the initial atom state data are initially set to target values as opposed to being stochastically sampled, e.g., as specified by ligand design data as described above with reference to step 1002 of FIG. 10A.

The encoder block of the denoising neural network can include any appropriate types of neural network layers (e.g., fully connected layers, convolutional layers, and so forth) in any appropriate number (e.g., 1 layer, 3, layers or 5 layers) and connected in any appropriate configuration (e.g., as a directed graph of layers). In a particular example, the encoder block includes a sequence of fully connected neural network layers and is configured to, for each atom, process data defining the 3D spatial position of the atom and the conditioning embedding for the atom to generate the atom embedding of the atom.

In some implementations, for each amino acid in the molecule complex, the system can generate one amino acid embedding for the amino acid that jointly represents all the atoms in the amino acid. Thus, in these implementations, the number of component embeddings may be equal to a sum of: (i) the number of atoms within the molecule complex that are not included within an amino acid in the molecule complex, and (ii) the number of amino acids in the molecule complex. The system can generate an amino acid embedding that jointly represents all the atoms in an amino acid in any appropriate way. For instance, the system can generate a respective atom embedding for each atom in the amino acid (as described above), and then combine the atom embeddings for the atoms in the amino acid, e.g., using a pooling operation (e.g., a max pooling or a summation pooling operation), or by processing the atom embeddings for the atoms in the amino acid using one or more neural network layers (e.g., fully connected layers or self-attention layers) to generate the amino acid embedding that jointly represents all the atoms in the amino acid. Generating amino acid embeddings that jointly represent all the atoms in an amino acid can significantly reduce the overall number of embeddings processed by the denoising neural network and thus reduce consumption of computational resources, e.g., memory and computing power, resulting from operations performed by an update block of the denoising neural network, as will be described next.

The system processes the component embeddings for the molecule components in the complex, using an update block of the denoising neural network, to generate a respective updated component embedding for each molecule component in the complex (1026). The update block of the denoising neural network can include a sequence of self-attention blocks. Each self-attention block can be configured to receive a respective current component embedding for each molecule component in the complex, to apply a self-attention operation to the current component embeddings of the molecule components in the complex, and to provide the updated component embeddings, e.g., for processing by a subsequent neural network layer.

Each self-attention block included in the update block can apply any appropriate self-attention operation to the current component embeddings of the molecule components in the complex, e.g., a single-head or multi-head query-key-value (QKV) self-attention operation. Optionally, the system can condition the self-attention operations of one or more of the self-attention blocks on the joint molecule embedding. An example process for implementing a self-attention operation conditioned on the joint molecule embedding is described in more detail with reference to FIG. 10C.

The update block of the denoising neural network can include any appropriate number of self-attention blocks (e.g., 1 self-attention block, or 10 self-attention blocks, or 50 self-attention blocks) and can optionally include additional neural network layers of any appropriate type (e.g., fully connected layers, convolutional layers, and so forth) in any appropriate number (e.g., 1 layer, 3, layers or 5 layers) and connected in any appropriate configuration (e.g., interleaved with the self-attention blocks).

The system processes the updated component embeddings (i.e., as generated by the update block of the denoising neural network) using a decoder block of the denoising neural network to generate the denoising output (1028). Examples of denoising outputs are described above with reference to FIG. 10A. The decoder block of the denoising neural network can include any appropriate types of neural network layers (e.g., fully connected layers, convolutional layers, and so forth) in any appropriate number (e.g., 1 layer, 3, layers or 5 layers) and connected in any appropriate configuration (e.g., as a directed graph of layers).

In a particular example, the decoder block can include a sequence of fully connected neural network layers that are configured to operate separately on each updated component embedding to generate a predicted error in the atom state data of each atom of the molecule complex at the current time step, or to generate predicted atom state data of each atom of the molecule complex, or both.

For example, in implementations where an amino acid embedding jointly represents all the atoms in an amino acid (as described above with reference to step 1024), the decoder block can process that (updated) amino acid embedding to generate respective denoising outputs for all the atoms in the amino acid. For instance, the decoder block can process an updated amino acid embedding that jointly represents all the atoms in an amino acid to generate a respective predicted error in the atom state data of each atom in the amino acid, or to generate respective atom state data of each atom in the amino acid, or both.

FIG. 10C is a flow diagram of an example process 1030 for updating a set of current component embeddings using a self-attention operation that is implemented by a self-attention block of the denoising neural network and that is conditioned on a joint molecule embedding. For convenience, the process 1030 will be described as being performed by a system of one or more computers located in one or more locations. For example, a molecule prediction system, e.g., the molecule prediction system 100 of FIG. 1, appropriately programmed in accordance with this specification, can perform the process 1030.

The system receives: (i) a set of current component embeddings, and (ii) a joint molecule embedding (1032). The set of current component embeddings includes a respective component embedding for each molecule component (e.g., atom, amino acid, etc.) in the complex. In some cases, for each amino acid in the molecule complex, all the atoms in the amino acid are jointly represented by an amino acid embedding for the amino acid, as described above with reference to FIG. 10B. The set of current component embeddings can be generated, e.g., by the encoder block of the denoising neural network or by a previous self-attention layer in the update block of the denoising neural network, as described above with reference to FIG. 10B. The joint molecule embedding can be generated by an embedding neural network, e.g., as described above with reference to FIG. 2B. The joint molecule embedding can include component-component embeddings for pairs of molecule components (e.g., amino acids, atoms, etc.) within the molecule complex, as described in more detail above with reference to FIG. 2B. For example, when the joint molecule embedding represents a protein and a ligand, the joint molecule embedding can include: (i) atom—atom embeddings, (ii) amino acid—amino acid embeddings, and (iii) amino acid—atom embeddings.

The system generates a set of intermediate attention scores based on the set of current component embeddings (1034). The set of intermediate attention scores includes a respective attention score for each pair of current component embeddings from the set of current component embeddings.

The system can generate the intermediate attention scores in any of a variety of possible ways. For instance, the system can generate a respective query embedding for each current component embedding by processing the current component embedding using a query neural network, e.g., as:

$$Q = {W}^{Q} \cdot E \qquad ( 3 )$$

where Q is a matrix where each column (or row) defines a respective query embedding, W^{Q} is a matrix of parameter values (defining the query neural network in this example), and E is a matrix where each column (or row) defines a respective current component embedding. Further, the system can generate a respective key embedding for each current component embedding by processing the current component embedding using a key neural network, e.g., as:

$$K = {W}^{K} \cdot E \qquad ( 4 )$$

where K is a matrix where each column (or row) defines a respective key embedding, W^{K} is a matrix of parameter values (defining the key neural network in this example), and E is a matrix where each column (or row) defines a respective current component embedding. The system can generate the intermediate attention scores based on the query embeddings and the key embeddings, e.g., as:

$$A = Q \cdot {K}^{T} \qquad ( 5 )$$

where A is a matrix of intermediate attention scores, Q is the matrix of query embeddings, and K is the matrix of key embeddings. In some implementations, to reduce computational costs, the system can determine the intermediate attention scores using optimized attention kernels, as described by Dao et al. in “FlashAttention: Fast and Memory-Efficient Exact Attention with IO-Awareness”.

The system generates a set of attention score biases based on the joint molecule embedding (1036). The set of attention score biases includes a respective attention score bias for each pair of current component embeddings from the set of current component embeddings.

The system can generate the set of attention score biases in any of a variety of possible ways. For instance, for each pair of current component embeddings, the system can generate the attention score bias for the pair of current component embeddings by processing a corresponding conditioning embedding selected from the collection of embeddings included in the joint molecule embedding using a projection neural network.

For a pair of current component embeddings that includes: (i) a first current atom embedding representing a first atom included in the molecule complex, and (ii) a second current atom embedding representing a second atom included in the molecule complex, the conditioning embedding can be the atom—atom embedding for the first atom and the second atom in the joint molecule embedding.

For a pair of current component embeddings that includes: (i) a first current atom embedding representing an atom included in an amino acid in the molecule complex, and (ii) a second current atom embedding representing an atom included in the molecule complex, the conditioning embedding can be the amino acid—atom embedding corresponding to: (i) the amino acid that includes the first atom, and (ii) the second atom, in the joint molecule embedding.

For a pair of current component embeddings that includes: (i) a first current atom embedding representing an atom included in a first amino acid in the molecule complex, and (ii) a second current atom embedding representing an atom included in a second amino acid in the molecule complex, the conditioning embedding can be the amino acid—amino acid embedding corresponding to: (i) the first amino acid, and (ii) the second amino acid, in the joint molecule embedding.

For a pair of current component embeddings that includes: (i) a first current amino acid embedding that jointly represents the atoms in a first amino acid in the molecule complex, and (ii) a second current amino acid embedding that jointly represents the atoms in a second amino acid in the molecule complex, the conditioning embedding can be the amino acid—amino acid embedding corresponding to: (i) the first amino acid, and (ii) the second amino acid, in the joint molecule embedding.

For a pair of current component embeddings that includes: (i) a current amino acid embedding that jointly represents the atoms in an amino acid in the molecule complex, and (ii) a current atom embedding that represents an atom included in the molecule complex, the conditioning embedding can be the amino acid—atom embedding corresponding to: (i) the amino acid, and (ii) the atom, in the joint molecule embedding.

The projection neural network can have any appropriate neural network architecture that enables the projection neural network to perform its described functions, e.g., processing a conditioning vector to generate an attention score bias. In particular, the projection neural network can include any appropriate number of neural network layers (e.g., 1 layer, or 5 layers, or 10 layers) in any appropriate number (e.g., 1 layer, 3, layers or 5 layers) and connected in any appropriate configuration (e.g., as a directed graph of layers).

The system generates a set of final attention scores by combining: (i) the intermediate attention scores, and (ii) the attention score biases (1038). The set of final attention scores includes a respective final attention score for each pair of current component embeddings in the set of current component embeddings. The system can generate the final attention score for a pair of current component embeddings by combining (e.g., summing): (i) the intermediate attention score for the pair of current component embeddings, and (ii) the attention score bias for the pair of current component embeddings. Optionally, the system can apply further processing operations to the set of final attention scores, e.g., by applying a soft-max operation to some or all of the final attention scores.

The system generates a set of updated component embeddings using: (i) the set of current component embeddings, and (ii) the set of final attention scores (1040). For instance, to generate the set of updated component embeddings, the system can generate a respective value embedding for each current component embedding in the set of current component embeddings by processing the current component embedding using a value neural network, e.g., as:

$$V = {W}^{V} \cdot E \qquad ( 6 )$$

where V is a matrix where each column (or row) defines a respective value embedding, W^{V} is a matrix of parameter values (defining the value neural network in this example), and E is a matrix where each column (or row) defines a respective current component embedding. The system can then generate the set of updated component embeddings, e.g., as:

$${E}^{l} = V \cdot A \qquad ( 7 )$$

where each column (or row) of E′ defines a respective updated component embedding, each column (or row) of V defines a respective value embedding, and A denotes the set of final attention scores arranged into a matrix.

In implementations where the self-attention block implements a multi-head attention operation, each head of the attention operation can individually perform the steps of the process 1030, and the updated component embeddings generated by each attention head can be combined (e.g., concatenated) to define the overall output of the multi-head attention operation. Each attention head can have a respective set of neural network parameters, having values that are specific to each attention head, that are used for generating the intermediate attention scores and the attention score biases.

FIG. 10D is a flow diagram of an example process 1050 generating gradients of a conditioning objective function with respect to current atom state data for the atoms in a molecule complex at a current denoising time step in a sequence of denoising time steps. For convenience, the process 1050 will be described as being performed by a system of one or more computers located in one or more locations. For example, a molecule prediction system, e.g., the molecule prediction system 100 of FIG. 1, appropriately programmed in accordance with this specification, can perform the process 1050.

The system receives current atom state data for some or all of the atoms in the complex at the current denoising time step in the sequence of denoising time steps over which the generative diffusion model denoises the current atom state data (1052). For example, when the molecule complex is a protein-ligand complex that includes a protein and a ligand, the system can receive current atom state data for some or all of the atoms in the ligand, and optionally, for some or all of the atoms in the protein. For each atom for which the system receives current atom state data, the system can receive the full set of current atom state data for the atom, or a subset of the full set of current atom state data for the atom.

The system processes the current atom state data using a property prediction neural network to generate a predicted value of a property of the molecule complex characterized by the current atom state data (1054). For example, when the molecule complex is a protein-ligand complex that includes a protein and a ligand, the predicted property can characterize, e.g., one or more of: a binding affinity of the ligand for the protein, absorption properties of the ligand, distribution properties of the ligand, metabolism properties of the ligand, excretion properties of the ligand, toxicity properties of the ligand, and so forth.

In some implementations, the property prediction neural network is configured to process atom state data for only a subset of the atoms in the molecule complex. For instance, predicting the binding affinity of a ligand for a protein may require the property prediction neural network to process current atom state data for some or all of the atoms in the protein as well as for the atoms in the ligand.

In some implementations, the property prediction neural network is configured to process a network input that includes both: (i) the current atom state data, and (ii) data identifying the current denoising time step in the sequence of denoising time steps over which the generative diffusion model denoises the atom state data for the atoms in the complex.

The property prediction neural network can have any appropriate neural network architecture that enables the property prediction neural network to perform its described functions, e.g., including processing atom state data for some or all of the atoms in the complex to generate a predicted value of a ligand property of the ligand. In particular, the property prediction neural network can include any appropriate types of neural network layers (e.g., fully connected layers, attention layers, pooling layers, convolutional layers, and so forth) in any appropriate number (e.g., 5 layers, 10 layers, or 20 layers) and connected in any appropriate configuration (e.g., as a directed graph of layers).

The system can train the property prediction neural network on a set of training examples using a machine learning training technique. Each training example in the set of training examples corresponds to a respective ligand and includes: (i) a training network input to the property prediction neural network, and (ii) an actual value of the property of the ligand.

For each training example, the training network input to the property prediction neural network can include: (i) respective atom state data for some or all of the atoms in a molecule complex for the training example, and optionally, (ii) data identifying a denoising time step in a sequence of denoising time steps. The system can generate the atom state data in the training network input by obtaining target (ground truth) atom state data for the atoms in the molecule complex, and then combining random noise with the target atom state data. The system can scale the random noise combined with the target atom state data by a constant that depends on the denoising time step, e.g., where the constant is defined by the same noise schedule implemented by the diffusion sampler of the generative diffusion model, as described with reference to step 1012 of FIG. 10A.

The system can train the property prediction neural network to optimize a loss function that, for each training example, measures a discrepancy between: (i) the actual value of the property specified by the training example, and (ii) a predicted value of the property that is generated by the property prediction neural network by processing the training network input of the training example. The loss function can measure the discrepancy in any appropriate way, e.g., using an absolute error metric or a squared error metric.

The system can train the property prediction neural network on the set of training examples using any appropriate machine learning training technique, e.g., using a stochastic gradient descent training technique.

As a particular example, the property prediction neural network can be the property prediction neural network 500 as described above with reference to FIG. 5A.

The system determines gradients of the conditioning objective function with respect to some or all of the current atom state data that was processed by the property prediction neural network to generate the predicted value of the property of the molecule complex (as described at step 1054) (1056). The conditioning objective function measures a discrepancy between: (i) the predicted value of the property, and (ii) a target (desired) value of the property. The conditioning objective function can measure the discrepancy using any appropriate error metric, e.g., using an absolute error metric or a squared error metric. The system can determine the gradients of the conditioning objective function using any appropriate technique, e.g., backpropagation.

The system provides the gradients of the conditioning objective function (1058), e.g., for generating a current estimate of the denoised atom state data for the atoms in the complex at the current denoising time step, as described above with reference to step 1008 of FIG. 10A.

FIG. 10E is a flow diagram of an example process 1060 for jointly training an embedding neural network and a generative diffusion model on a training example. For convenience, the process 1060 will be described as being performed by a system of one or more computers located in one or more locations. For example, a molecule prediction system, e.g., the molecule prediction system 100 of FIG. 1, appropriately programmed in accordance with this specification, can perform the process 1060.

The system generates a joint molecule embedding for the training example by processing molecule data included in a training input of the training example using the embedding neural network (1062).

As part of generating the joint molecule embedding, the embedding neural network can generate a design embedding that represents any molecule design criteria provided in the input to the embedding neural network, e.g., as described above with reference to FIG. 7C. The design embedding can include a respective atom embedding representing each ligand atom in a set of possible ligand atoms that are eligible for inclusion in a ligand of the molecule complex. The number of ligand atoms represented in the design embedding define the maximum number of atoms that the system can select for inclusion in the ligand of the molecule complex. As part of generating the joint molecule embedding, the system can cause the embedding neural network to generate a design embedding that includes a number of atom embeddings that is at least as great as the number of atoms included in the ligand of the training example.

For each atom in the molecule complex, the system generates target atom state data for the atom based on the set of atom features of the atom (1064). The set of atom features of the atom can include continuous features, e.g., that define the 3D spatial position of the atom and/or the partial charge of the atom, and/or categorical features, e.g., that define the hybridization state and element type of the atom. For each continuous feature in the atom state data of the atom, the system can populate one or more corresponding dimensions of the target atom state data for the atom with numerical values defining the continuous feature. For each categorical feature in the atom state data, the system can populate a set of corresponding dimensions of the target atoms state data for the atom with a distribution over a set of possible values of the categorical feature, e.g., a one-hot distribution over the set of possible values of the categorical feature that uniquely identifies the actual value of the categorical feature.

If the number of ligand atoms in the set of possible ligand atoms is greater than the actual number of atoms included in a ligand of the complex, then the set of possible ligand atoms includes one or more “extra” ligand atoms that will not be selected for inclusion in the ligand of the complex. For each extra ligand atom, the system can generate target atom state data that defines the 3D spatial position of the atom as being a predefined “throw-away” position. The system can set the other dimensions of the target atom state data for the extra ligand atom (i.e., the dimensions that do not define the 3D spatial position of the extra ligand atom) to masked (default) values.

The system samples a time step from the sequence of denoising time steps (1066). More specifically, during inference, the generative diffusion model can be configured to perform a sequence of denoising time steps, e.g., as described with reference to steps 1004-1012 of FIG. 10A. During training, the system can randomly sample a single denoising time step from the sequence of denoising time steps, e.g., in accordance with a uniform distribution over the sequence of denoising time steps.

The system generates respective noisy atom state data for each atom in the complex by combining random noise with the target atom state data of the atom (1068). For instance, for each atom in the complex, the system can generate the noisy atom state data for the atom by adding random noise to the target atom state data of the atom. The system can scale the random noise combined with the target atom state data of the atoms by a constant that depends on the sampled time step, e.g., where the values of the constants corresponding to the denoising time steps are defined by a noise schedule. Optionally, the system can refrain from combining random noise with any dimensions of the target atom state data for an atom that are designated as being scaffolding data.

The system generates a denoising output using the denoising neural network while the denoising neural network is conditioned on the joint molecule embedding (1070). An example process for generating a denoising output is described in detail with reference to FIG. 10B. At step 1022 of FIG. 10B, the current atom state data for each atom in the complex can be defined as the noisy atom state data for each atom in the complex.

The system determines gradients of an objective function that depends on the denoising output and uses the gradients to update the parameter values of the denoising neural network and the embedding neural network (1072). The objective function can measure an error between: (i) the denoising output of the denoising neural network, and (ii) a target output of the denoising neural network. The target output of the denoising neural network can define an output of the denoising neural network that, if used to generate a current estimate of the atom state data of the atoms in the complex (as described in step 1008 of FIG. 10A), would cause the current estimate of the atom state data of the atoms to match the target atom state data of the atoms in the molecule complex of the training example.

In some cases, the dimensions of the target atom state data for the extra ligand atoms (i.e., that are not included in a ligand of the molecule complex) other than those that define the 3D spatial position of the extra ligand atoms (i.e., as the throw-away position) are set to masked values. The system can exclude the masked dimensions of the target atom state data for the extra ligand from the computation of the objective function, i.e., such that the objective function is independent of the values of the masked dimensions of the target atom state data.

In general, in implementations of the above described techniques a protein and a ligand for the protein can be a receptor or enzyme and the ligand can be an agonist or antagonist of the receptor or enzyme; or the protein can be a cell surface marker and the ligand can be an antibody or aptamer or a label such as a fluorescent label, which binds to the cell surface marker (e.g. for identifying or treating cancerous cells). As another example, the protein can be associated with a disease and the ligand can be an antibody or aptamer marker that binds to, i.e. that recognizes, the protein, for diagnostic purposes. The protein and/or ligand, and in general a molecule or molecule complex as described herein, can be a human, animal, or plant molecule, molecule complex, protein and/or ligand; and any of these can have been derived from human, animal, or plant, e.g. from a sample from a human, animal, or plant.

Some example sources of training data that can be used to train implementations of the system are now described.

In general the training data corresponds to the predictions made—for example 3D structure data of molecules or molecule complexes for a structure prediction neural network; examples of bound ligands and/or binding affinity data for a molecule design neural network such as a ligand design neural network; distograms for a distogram prediction neural network; contact probabilities for a contact probability prediction neural network; property data such as ADMET data where a property is predicted; and so forth.

In general such data can be obtained by physical experiments and/or using computational methods. Whilst this may initially be slow, an advantage of the described techniques is that, once trained, new information can be obtained quickly.

Also or instead training data can be obtained from a wide range of public or commercial databases. The previously cited papers reference some examples; a few other examples are given below.

For a molecule design neural network such as a ligand design neural network suitable training data can be obtained from, e.g.: ChEMBL (https://www.ebi.ac.uk/chembl/), which is a manually curated database of bioactive molecules with drug-like properties; BioLip (Biological Ligand-Protein Interaction Database), which is a ligand-protein binding database; DUD-E (Directory of Useful Decoys—Enhanced, where decoys are molecules unlikely to bind but with similar physical properties to actives; https://dude.docking.org/); SitesBase (a database for structure-based protein-ligand binding site comparisons).

For binding affinity/binding energy suitable training data can be obtained from, e.g.: PDBbind (experimentally measured binding affinity data (Kd, Ki, IC50) for protein-ligand complexes that are deposited in the PDB) BindingDB; ChEMBL; and ExCAPE-DB.

For property data such as ADMET data suitable training data can be obtained from, e.g.: ChEMBL, PubChem (NCBI, the National Center for Biotechnology Information); or DrugBank. Property data can also be obtained from computational tools such as ADMETlab 2.0, and pkCSM.

For contact maps suitable training data can be obtained from, e.g.: the 4D Nucleome Data Portal (Reiff et al., “The 4D Nucleome Data Portal as a resource for searching and visualizing curated nucleomics data”, Nature Communications 13, 2365, 2022) and/or from Hi-C experiments.

Generally, the Protein Data Bank (PDB, wwpdb.org) includes a very large amount of useful data including 3D structures (it could be used to obtain distograms), and chemical and biological information, including via the Chemical Component Dictionary, CCD). Other sources of training data include UniProt (Universal Protein Resource) for protein sequence and functional information; and BRENDA, SABIO-RK (for enzyme information and reaction kinetics.

This specification uses the term “configured” in connection with systems and computer program components. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non-transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array) or an ASIC (application-specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand-alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a data communication network.

In this specification the term “engine” is used broadly to refer to a software-based system, subsystem, or process that is programmed to perform one or more specific functions. Generally, an engine will be implemented as one or more software modules or components, installed on one or more computers in one or more locations. In some cases, one or more computers will be dedicated to a particular engine; in other cases, multiple engines can be installed and running on the same computer or computers.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone that is running a messaging application, and receiving responsive messages from the user in return.

Data processing apparatus for implementing machine learning models can also include, for example, special-purpose hardware accelerator units for processing common and compute-intensive parts of machine learning training or production, i.e., inference, workloads.

Machine learning models can be implemented and deployed using a machine learning framework, e.g., a TensorFlow framework, or a Jax framework.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface, a web browser, or an app through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

In addition to the embodiments described above, the following embodiments are also innovative:

Embodiment 1 is a method performed by one or more computers, the method comprising:
obtaining molecule data characterizing a molecule;
processing a network input comprising the molecule data using an embedding neural network to generate a molecule embedding representing the molecule;
processing the molecule embedding representing the molecule using a prediction machine learning model to generate an output prediction characterizing the molecule;
wherein the embedding neural network has been jointly trained along with one or more, in particular a plurality of prediction neural networks that are each configured to perform a respective prediction task; and, for example
wherein the plurality of prediction neural networks comprise a ligand design neural network that is configured to perform a ligand design task by operations comprising:
   receiving an input molecule embedding that represents at least a portion of a protein and that is generated by the embedding neural network; and
   processing the input molecule embedding to generate predicted ligand data defining a predicted ligand that is predicted to bind to the protein.

Embodiment 2 is the method of embodiment 1, wherein:
the embedding neural network comprises one or more molecule embedding neural networks; and
the embedding neural network is configured to process molecule data characterizing a first molecule and a second molecule by performing operations comprising:
   processing the molecule data characterizing the first molecule and the second molecule using the one or more molecule embedding neural network to generate a molecule embedding of the first molecule;
   processing the molecule data characterizing the first molecule and the second molecule using the one or more molecule embedding neural network to generate a molecule embedding of the second molecule; and
   processing the molecule embedding of the first molecule and the molecule embedding of the second molecule to generate a molecule embedding characterizing the first molecule and the second molecule.

Embodiment 2 is the method of embodiment 2, wherein:
the molecule embedding of the first molecule comprises a respective component embedding for each of one or more molecular components of the first molecule;
the molecule embedding of the second molecule comprises a respective component embedding for each of one or more molecular components of the second molecule;
processing the molecule embedding of the first molecule and the molecule embedding of the second molecule to generate the molecule embedding characterizing the first molecule and the second molecule comprises:
   generating data defining a 1D sequence of component embeddings by concatenating: (i) the component embeddings for the molecular components of the first molecule, and (ii) the component embeddings for the molecular components of the second molecule;
wherein the molecule embedding characterizing the first molecule and the second molecule is derived from the 1D sequence of component embeddings.

Embodiment 4 is the method of embodiment 3, wherein processing the molecule embedding of the first molecule and the molecule embedding of the second molecule to generate the molecule embedding characterizing the first molecule and the second molecule further comprises:
transforming the 1D sequence of component embeddings into a two-dimensional (2D) array of embeddings;
wherein the molecule embedding characterizing the first molecule and the second molecule is derived from the 2D array of embeddings.

Embodiment 5 is the method of embodiment 3 or embodiment 4, wherein the wherein the 1D sequence of component embeddings comprises a respective atom embedding for each of a plurality of atoms in the first molecule or the second molecule.

Embodiment 6 is the method of embodiment 5, wherein 2D array of embeddings comprises a plurality of atom—atom embeddings that are each derived from a respective pair of atom embeddings within the 1D sequence of component embeddings.

Embodiment 7 is the method of any one of embodiments 3-6, wherein the 1D sequence of component embeddings comprises a respective amino acid embedding for each of a plurality of amino acids in the first molecule or the second molecule.

Embodiment 8 is the method of embodiment 7, wherein the 2D array of embeddings comprises a plurality of amino acid-amino acid embeddings that are each derived from a respective pair of amino acid embeddings within the 1D sequence of component embeddings.

Embodiment 9 is the method of any one of embodiment 7 or embodiment 8, when including the method of embodiment 5, wherein the 2D array of embeddings comprises a plurality of amino acid—atom embeddings that are each derived from: (i) a respective atom embedding within the 1D sequence of component embeddings, and (ii) a respective amino acid embedding within the 1D sequence of component embeddings.

Embodiment 10 is the method of any one of embodiments 4-9, wherein transforming the 1D sequence of component embeddings into the 2D array of embeddings comprises:
applying an outer product operation to the 1D sequence of component embeddings; or
applying a 2D concatenation operation to the 1D sequence of component embeddings.

Embodiment 11 is the method of any one of embodiments 4-10, wherein the embedding neural network further comprises a fusion neural network; and
wherein processing the molecule embedding of the first molecule and the molecule embedding of the second molecule to generate the molecule embedding characterizing the first molecule and the second molecule further comprises:
   processing the 2D array of embeddings using the fusion neural network to generate an updated 2D array of embeddings;
   wherein the updated 2D array of embeddings defines the molecule embedding characterizing the first molecule and the second molecule.

Embodiment 12 is the method of embodiment 11, wherein the fusion neural network comprises a sequence of self-attention blocks, wherein each self-attention block is configured to perform operations comprising:
apply one or more self-attention operations to an input 2D array of embedding to update the input 2D array of embeddings.

Embodiment 13 is the method of embodiment 12, wherein for one or more of the self-attention blocks, the self-attention operations comprise one or more row-wise self-attention operations.

Embodiment 14 is the method of any one of embodiments 12-13, wherein for one or more of the self-attention blocks, the self-attention operations comprise one or more column-wise self-attention operations.

Embodiment 15 is the method of any one of embodiments 12-14, wherein for one or more of the self-attention blocks, the self-attention operations comprise one or more triangle self-attention operations.

Embodiment 16 is the method of any one of embodiments 1-15, wherein the plurality of prediction neural networks comprises a property prediction neural network that is configured to:
receive an input molecule embedding that represents a first molecule and a second molecule; and
generate a property score that defines a predicted joint property of the first molecule and the second molecule using the input molecule embedding.

Embodiment 17 is the method of embodiment 16, wherein the property prediction neural network and the embedding neural network have been jointly trained on a plurality of training examples, wherein each training example comprises: (i) a training input that characterizes a first molecule and a second molecule for the training example, and (ii) a target property score that defines a property of the first molecule and the second molecule for the training example.

Embodiment 18 is the method of embodiment 17, wherein the joint training of the property prediction neural network and the embedding neural network on the plurality of training examples comprises, for each training example:
processing the training input of the training example using the embedding neural network to generate a molecule embedding for the training example representing the first molecule and the second molecule for the training example;
processing the molecule embedding for the training example using the property prediction neural network to generate a predicted property score for the first molecule and the second molecule for the training example; and
backpropagating gradients of an objective function through the property prediction neural network and into the embedding neural network, wherein the objective function measures a discrepancy between: (i) the target property score specified by the training example, and (ii) the predicted property score generated by the embedding neural network and the property prediction neural network for the training example.

Embodiment 19 is the method of embodiment 16, wherein the property prediction neural network is further configured to:
obtain a predicted joint three-dimensional (3D) structure of the first molecule and the second molecule; and
generate the property score that defines the predicted joint property of the first molecule and the second molecule using the input molecule embedding and the predicted joint 3D structure of the first molecule and the second molecule.

Embodiment 20 is the method of embodiment 19, wherein obtaining the predicted joint 3D structure of the first molecule and the second molecule comprises:
generating, while conditioned on the input molecule embedding, the predicted joint 3D structure of the first molecule and the second molecule using a generative model.

Embodiment 21 is the method of embodiment 20, wherein the embedding neural network and the generative model have been jointly trained on a plurality of training examples, wherein each training example comprises: (i) a training input that characterizes a first molecule and a second molecule for the training example, and (ii) a target output based on a joint 3D structure of the first molecule and the second molecule for the training example.

Embodiment 22 is the method of embodiment 21, wherein the joint training of the embedding neural network and the generative model on the plurality of training examples comprises, for each training example:
processing the training input of the training example using the embedding neural network to generate a molecule embedding for the training example representing the first molecule and the second molecule for the training example;
processing the molecule embedding for the training example using the generative model to generate a predicted output characterizing a predicted joint 3D structure of the first molecule and the second molecule for the training example; and
backpropagating gradients of an objective function through the generative model and into the embedding neural network, wherein the objective function measures a discrepancy between: (i) the target output specified by the training example, and (ii) the predicted output generated by the embedding neural network and the generative model for the training example.

Embodiment 23 is the method of any one of embodiments 19-22, wherein the predicted joint 3D structure of the first molecule and the second molecule defines a respective predicted three-dimensional spatial location of each atom in the first molecule and of each atom in the second molecule.

Embodiment 24 is the method of embodiment 23, wherein generating the property score that defines the predicted joint property of the first molecule and the second molecule using the input molecule embedding and the predicted joint 3D structure of the first molecule and the second molecule comprises:
generating data defining a graph representing at least a portion of the predicted joint 3D structure of the first molecule and the second molecule; and
processing the graph representing at least the portion of the predicted joint 3D structure of the first molecule and the second molecule using a graph neural network to generate the property score that defines the predicted joint property of the first molecule and the second molecule.

Embodiment 25 is the method of embodiment 24, wherein the graph neural network comprises a plurality of message passing layers.

Embodiment 26 is the method of any one of embodiments 24-25, wherein the graph representing at least the portion of the predicted joint 3D structure of the first molecule and the second molecule comprises: (i) a sets of nodes, and (ii) a set of edges, wherein:
the set of nodes comprises a plurality of atom nodes that each represent a respective atom in the first molecule or in the second molecule; and
each edge connects a respective pair of nodes.

Embodiment 27 is the method of embodiment 26, wherein generating the data defining the graph representing at least the portion of the predicted joint 3D structure of the first molecule and the second molecule comprises:
generating the set of edges based at least in part on 3D spatial distances between pairs of atoms in the first molecule and in the second molecule.

Embodiment 28 is the method of embodiment 27, wherein generating the set of edges based at least in part on 3D spatial distances between pairs of atoms in the first molecule and in the second molecule comprises, for each pair of atoms that comprises a respective first atom in the first molecule or in the second molecule and a respective second atom in the first molecule or in the second molecule:
determining that an atom node representing the first atom and an atom node representing the second atom should be connected by an edge if a 3D spatial distance between the first atom and the second atom satisfies a threshold.

Embodiment 29 is the method of any one of embodiments 26-28, wherein the set of nodes in the graph further comprises a plurality of super nodes, wherein the plurality of super nodes comprises a respective super node representing each of a plurality of amino acid residues in the first molecule and the second molecule; and
wherein the set of edges in the graph comprises, for each atom node in the graph that represents an atom in the protein, a respective edge between the atom node and a corresponding super node representing an amino acid residue that includes the atom.

Embodiment 30 is the method of embodiment 29, wherein the first molecule and the second molecule comprise a plurality of structural motifs, and wherein the plurality of super nodes further comprises a respective super node representing each structural motif in the first molecule and the second molecule; and
wherein the set of edges in the graph comprises, for each atom node in the graph that represents an atom in the first molecule and the second molecule, a respective edge between the atom node and a corresponding super node representing a structural motif that includes the atom.

Embodiment 31 is the method of embodiment 30, wherein the set of edges comprises a respective edge between each pair of super nodes from the plurality of super nodes included in the graph.

Embodiment 32 is the method of any one of embodiments 20-31, wherein the generative model is a generative diffusion model that comprises a denoising neural network.

Embodiment 33 is the method of embodiment 32, wherein generating the property score that defines the predicted joint property of the first molecule and the second molecule using the input molecule embedding comprises:
generating positional data defining a respective initial position of each atom in a complex comprising the first molecule and the second molecule;
generating property data defining an initial predicted joint property of the first molecule and the second molecule;
denoising the positional data and the property data over a sequence of time steps using the denoising neural network and while the denoising neural network is conditioned on the input molecule embedding;
wherein, after a final time step in the sequence of time steps:
   the positional data defines the predicted joint 3D structure of the first molecule and the second molecule; and
   the property data defines the property score.

Embodiment 34 is the method of embodiment 33, wherein denoising the positional data and the property data over the sequence of time steps using the denoising neural network and while the denoising neural network is conditioned on the input molecule embedding comprises, at each of one or more time steps in the sequence of time steps:
receiving current positional data that defines a respective current position of each atom in the complex at the time step;
receiving current property data that defines a current predicted joint property of the first molecule and the second molecule;
generating a denoising output using the denoising neural network and while the denoising neural network is conditioned on the input molecule embedding; and
generating positional data that defines a respective position of each atom in the complex at a next time step using the denoising output; and
generating property data that defines a predicted joint property of the first molecule and the second molecule at the next time step using the denoising output.

Embodiment 35 is the method of embodiment 34, wherein the denoising output comprises: (i) a respective predicted error in the current position of each atom in the complex at the time step, and (ii) a respective predicted error in the current predicted property score of the first molecule and the second molecule at the time step.

Embodiment 36 is the method of any one of embodiments 34-35, wherein generating the denoising output using the denoising neural network and while the denoising neural network is conditioned on the input molecule embedding comprises:
generating a set of embeddings using an encoder block of the denoising neural network, wherein the set of embeddings comprises:
   a plurality of atom embeddings, wherein each atom embedding represents one or more respective atoms in the complex and is based at least in part on the respective current spatial position of the one or more atoms at the time step; and
   a property embedding that is based at least in part on the current predicted joint property of the first molecule and the second molecule at the time step;
processing the set of embeddings using an update block of the denoising neural network to generate a set of updated embeddings; and
processing the set of updated embeddings to generate the denoising output.

Embodiment 37 is the method of embodiment 36, wherein the update block of the denoising neural network comprises a sequence of self-attention blocks;
wherein each of the self-attention blocks are configured to:
   receive a set of current embeddings that comprises a plurality of current atom embeddings and a current property score embedding; and
   apply one or more self-attention operations to the set of current embeddings to update the set of current embeddings;
wherein each of the one or more self-attention operations are conditioned on the input molecule embedding.

Embodiment 38 is the method of any one of embodiments 16-37, wherein the property score is a binding affinity score that defines a predicted binding affinity of the first molecule and the second molecule, and wherein generating the property score that defines the predicted joint property of the first molecule and the second molecule using the input molecule embedding comprises:
conditioning the generation of the property score on data specifying a type of binding affinity assay; and
wherein the property score defines the predicted binding affinity of the first molecule and the second molecule as measured by the specified type of binding affinity assay.

Embodiment 39 is the method of embodiment 38, wherein type of binding affinity assay is: a surface plasmon resonance (SPR) assay, or an isothermal titration calorimetry (ITC) assay, or a fluorescence polarization (FP) assay, or an enzyme-linked immunosorbent assay (ELISA), or a radioligand binding assay, or a bioluminescence resonance energy transfer (BERT) assay.

Embodiment 40 is the method of any one of embodiments 16-39, wherein the property score defines one or more of: a likelihood of a binding event that involves the protein and the ligand; or a binding affinity of the protein and the ligand; or a likelihood that the ligand is an agonist for the protein; or a likelihood that the ligand is an antagonist for the protein; or a predicted potency of the ligand when acting on the protein; or a predicted inhibitory effect of the ligand when acting on the protein.

Embodiment 41 is the method of any one of embodiments 1-40, wherein the plurality of prediction neural networks comprises a structure prediction neural network that is configured to:
receive an input molecule embedding that represents a first molecule and a second molecule; and
generate a predicted joint three-dimensional (3D) structure of the first molecule and the second molecule using the input molecule embedding,
   wherein the predicted joint 3D structure of the first molecule and the second molecule defines a respective predicted three-dimensional spatial location of each atom in the first molecule and of each atom in the second molecule.

Embodiment 42 is the method of any embodiment 41, wherein the structure prediction neural network is a generative diffusion model that comprises a denoising neural network.

Embodiment 43 is the method of embodiment 42, wherein generating the predicted joint three-dimensional (3D) structure of the first molecule and the second molecule using the input molecule embedding comprises:
generating positional data defining a respective initial position of each atom in a complex comprising the first molecule and the second molecule;
denoising the positional data over a sequence of time steps using the denoising neural network and while the denoising neural network is conditioned on the input molecule embedding;
wherein the predicted joint 3D structure of the first molecule and the second molecule is defined by the positional data after a final time step in the sequence of time steps.

Embodiment 44 is the method of embodiment 43, wherein generating positional data defining the respective initial position of each atom in the complex comprises:
sampling the respective initial position of each atom in the complex from a probability distribution over 3D space.

Embodiment 45 is the method of any one of embodiments 43-44, wherein denoising the positional data over the sequence of time steps using the denoising neural network and while the denoising neural network is conditioned on the input molecule embedding comprises, at each of one or more time steps in the sequence of time steps:
receiving current positional data that defines a respective current position of each atom in the complex at the time step;
generating a denoising output using the denoising neural network and while the denoising neural network is conditioned on the input molecule embedding; and
generating positional data that defines a respective position of each atom in the complex at a next time step using the denoising output.

Embodiment 46 is the method of embodiment 45, wherein the denoising output comprises a respective predicted error in the current position of each atom in the complex at the time step.

Embodiment 47 is the method of any one of embodiments 45-46, wherein generating the denoising output using the denoising neural network and while the denoising neural network is conditioned on the input molecule embedding comprises:
generating a set of component embeddings using an encoder block of the denoising neural network, wherein each component embedding represents one or more atoms in the complex and is based at least in part on the respective current spatial position of the one or more atoms at the time step;
processing the set of component embeddings using an update block of the denoising neural network to generate a set of updated atom embeddings; and
processing the set of updated component embeddings to generate the denoising output.

Embodiment 48 is the method of embodiment 47, wherein the set of component embeddings includes a respective atom embedding representing each of a plurality of atoms in the complex.

Embodiment 49 is the method of embodiment 47 or embodiment 48, wherein the set of component embeddings includes, for each of a plurality of amino acids in the complex, a respective amino acid embedding that jointly represents all of the atoms included in the amino acid.

Embodiment 50 is the method of any one of embodiments 47-49, wherein each component embedding in the set of component embeddings is based on, for each of the one or more atoms represented by the component embedding: (i) the current position of the atom at the time step, and (ii) a respective conditioning embedding for the atom that is selected from a collection of embeddings included in the input molecule embedding.

Embodiment 51 is the method of embodiment 50, wherein for each component embedding that represents an atom in the complex, the conditioning embedding for the atom comprises an atom—atom embedding corresponding to the atom in the input molecule embedding.

Embodiment 52 is the method of any one of embodiments 50-51, wherein for each component embedding that represents an atom in the complex that is included in an amino acid in the complex, the conditioning embedding for the atom comprises an amino acid—amino acid embedding corresponding to the amino acid in the input molecule embedding.

Embodiment 53 is the method of any one of embodiments 47-52, wherein the update block of the denoising neural network comprises a sequence of self-attention blocks;
wherein each of the self-attention blocks are configured to apply one or more self-attention operations to a set of current component embeddings to update the set of current component embeddings;
wherein each of the one or more self-attention operations are conditioned on the input molecule embedding.

Embodiment 54 is the method of embodiment 53, wherein applying a self-attention operation to the set of current component embeddings to update the set of current component embeddings comprises:
generating, based on the current set of component embeddings, a respective intermediate attention score for each pair of current component embeddings from the set of current component embeddings;
generating, based on the input molecule embedding, a respective attention score bias for each pair of current component embeddings from the set of current component embeddings;
generating a respective final attention score for each pair of current component embeddings from the set of current component embeddings based on the intermediate attention scores and the attention score biases; and
updating the set of current component embeddings using the final attention scores.

Embodiment 55 is the method of embodiment 54, wherein for each pair of current component embeddings from the set of current component embeddings, generating the attention score bias for the pair of current component embeddings comprises:
summing the intermediate attention score for the pair of current component embeddings and the attention score bias for the pair of current component embeddings.

Embodiment 56 is the method of any one of embodiments 54-55, wherein for each pair of current component embeddings from the set of current component embeddings, generating the attention score bias for the pair of current component embeddings comprises:
processing a respective conditioning embedding selected from a collection of embeddings included in the input molecule embedding using a projection neural network to generate the attention score bias.

Embodiment 57 is the method of embodiment 56, wherein for each pair of current component embeddings that includes: (i) a first atom embedding representing a first atom in the complex, and (ii) a second atom embedding representing a second atom included in the complex, the selected conditioning embedding comprises an atom—atom embedding corresponding to the first atom and the second atom in the input molecule embedding.

Embodiment 58 is the method of any one of embodiments 56-57, wherein for each pair of current component embeddings that includes: (i) a first atom embedding representing a first atom included in an amino acid in the complex, and (ii) a second atom embedding representing a second atom included in the complex, the selected conditioning embedding comprises an amino acid—atom embedding corresponding to: (i) the amino acid that includes the first atom, and (ii) the second atom, in the input molecule embedding.

Embodiment 59 is the method of any one of embodiments 56-58, wherein for each pair of current component embeddings that includes: (i) a first atom embedding representing a first atom included in a first amino acid in the complex, and (ii) a second atom embedding representing a second atom included in a second amino acid in the complex, the selected conditioning embedding comprises an amino acid—amino acid embedding corresponding to: (i) the first amino acid, and (ii) the second amino acid, in the input molecule embedding.

Embodiment 60 is the method of any one of embodiments 56-59, wherein for each pair of current component embeddings that includes: (i) a first amino acid embedding representing a first amino acid in the complex, and (ii) a second amino acid embedding representing a second amino acid in the complex, the selected conditioning embedding comprises an amino acid—amino acid embedding corresponding to: (i) the first amino acid, and (ii) the second amino acid, in the input molecule embedding.

Embodiment 61 is the method of any one of embodiments 56-60, wherein for each pair of current component embeddings that includes: (i) an amino acid embedding representing an amino acid in the complex, and (ii) an atom embedding that represents an atom included in the complex, the selected conditioning embedding comprises an amino acid—atom embedding corresponding to: (i) the amino acid, and (ii) the atom, in the input molecule embedding.

Embodiment 62 is the method of any one of embodiments 1-61, wherein the input molecule embedding jointly represents the protein and ligand design criteria that define desired characteristics of the predicted ligand.

Embodiment 63 is the method of embodiment 62, wherein the embedding neural network and the ligand design neural network are jointly trained to optimize an objective function that encourages the embedding neural network and the ligand design neural network to attempt to generate ligand data defining a ligand that binds to the protein and that satisfies the ligand design criteria.

Embodiment 64 is the method of any one of embodiments 62-63, wherein the ligand design criteria define a respective target value for each of one or more global properties of the ligand that characterize the ligand as a whole.

Embodiment 63 is the method of embodiment 64, wherein the ligand design criteria define a respective target value for a set of global properties of the ligand characterizing one or more of: a binding affinity of the ligand for the protein, absorption properties of the ligand, distribution properties of the ligand, metabolism properties of the ligand, excretion properties of the ligand, toxicity properties of the ligand, a number of rings in the ligand, a molecular weight of the ligand, a lipophilicity (logP) of the ligand, an ability of the ligand to donate or accept hydrogen bonds, a total polar surface area of the ligand, a number of rotatable bonds in the ligand, a number of chiral centers (stereocenters) in the ligand, a number of electrophilic (electron-accepting) centers in the ligand, a number of nucleophilic (electron-donating) centers in the ligand.

Embodiment 66 is the method of any one of embodiments 62-65, wherein the ligand design criteria define a respective target value for each of one or more atom-specific properties of the ligand that each relate to a specific atom in the ligand.

Embodiment 67 is the method of embodiment 66, wherein the ligand design criteria define a respective target value for a set of atom-specific properties of the ligand that, for each of one or more atoms in the ligand, characterize one or more of: an elemental type of the atom, a hybridization state of the atom, or a partial charge of the atom.

Embodiment 68 is the method of any one of embodiments 62-67, wherein the ligand design criteria comprises protein scaffolding data, or ligand scaffolding data, or both.

Embodiment 69 is the method of embodiment 68, wherein the protein scaffolding data defines a respective target three-dimensional (3D) spatial position of each of one or more atoms in the protein when in a complex with the ligand.

Embodiment 70 is the method of any one of embodiments 68-69, wherein the ligand scaffolding data defines a respective target 3D spatial position of each of one or more atoms in the ligand when in a complex with the protein.

Embodiment 71 is the method of any one of embodiments 62-70, wherein the embedding neural network comprises a protein embedding neural network and a design embedding neural network, and wherein the embedding neural network is configured to process the ligand design criteria and protein data representing the protein to generate the input molecule embedding jointly representing the protein and ligand design criteria by performing operations comprising:
processing the protein data using the protein embedding neural network to generate a protein embedding of the protein;
processing the ligand design criteria using the design embedding neural network to generate a design embedding of the ligand design criteria; and
processing the protein embedding and the design embedding to generate the input molecule embedding jointly representing the protein and the ligand design criteria.

Embodiment 72 is the method of embodiment 71, wherein processing the ligand design criteria using the design embedding neural network to generate the design embedding of the ligand design criteria comprises:
generating a collection of initial atom embeddings based on the ligand design criteria, wherein the collection of initial atom embeddings includes a respective atom embedding for each ligand atom in a set of possible ligand atoms that are eligible for inclusion in the ligand;
processing the collection of initial atom embeddings, by a plurality of neural network layers of the design embedding neural network, to generate a collection of final atom embeddings that includes a respective final atom embedding for each ligand atom in the set of possible ligand atoms;
wherein the collection of final atom embeddings defines the design embedding of the ligand design criteria.

Embodiment 73 is the method of embodiments 72, wherein the ligand design criteria specify a respective target value for each of one or more global properties of the ligand; and
wherein generating the collection of initial atom embeddings based on the ligand design criteria comprises:
   including the respective target value for each of the one or more global properties of the ligand in each initial atom embedding in the collection of initial atom embeddings.

Embodiment 74 is the method of any one of embodiments 72-73, wherein the ligand design criteria specify a respective target value for each of one or more atom-specific properties of the ligand; and
wherein generating the collection of initial atom embeddings based on the ligand design criteria comprises, for each atom-specific property of the ligand:
   including the target value of the atom-specific property only in the initial atom embedding representing the ligand atom that is characterized by the atom-specific property.

Embodiment 75 is the method of any one of embodiments 72-74, wherein a number of ligand atom embeddings in the set of ligand atom embeddings defines a maximum number of atoms that can be selected for inclusion in the ligand.

Embodiment 76 is the method of any one of embodiments 71-75, wherein the plurality of neural network layers of the design embedding neural network include one or more self-attention neural network layers.

Embodiment 77 is the method of any one of embodiments 72-76, wherein the ligand design criteria leave undefined at least part of a chemical structure of the ligand.

Embodiment 78 is the method of any one of embodiments 71-77, wherein the protein embedding comprises a respective amino acid embedding of each amino acid in the protein;
wherein the design embedding comprises a respective atom embedding of each ligand atom in a set of ligand atoms that are eligible for inclusion in the ligand, wherein a number of ligand atom embeddings in the set of ligand atom embeddings defines a maximum number of atoms that can be selected for inclusion in the ligand; and
wherein processing the protein embedding and the design embedding to generate the input molecule embedding jointly representing the protein and the ligand design criteria comprises:
   generating data defining a 1D sequence of amino acid embeddings and atom embeddings by concatenating: (i) the amino acid embeddings of the protein embedding, and (ii) the ligand atom embeddings of the design embedding;
   wherein the input molecule embedding jointly representing the protein and the ligand design criteria is derived from the 1D sequence of amino acid embeddings and atom embeddings.

Embodiment 79 is the method of any one of embodiments 71-78, wherein the ligand design neural network is a generative diffusion model that comprises a denoising neural network.

Embodiment 80 is the method of embodiment 79, wherein generating, using the input molecule embedding, predicted ligand data defining a predicted ligand that is predicted to bind to the protein comprises:
generating respective atom state data for each atom in the protein and for each ligand atom in a set of ligand atoms that are eligible for inclusion in the ligand;
denoising the atom state data over a sequence of time steps using the denoising neural network and while the denoising neural network is conditioned on the input molecule embedding jointly representing the protein and the ligand design criteria; and
generating the predicted ligand data based on the atom state data after a final time step in the sequence of time steps.

Embodiment 81 is the method of embodiment 80, wherein for each ligand atom in the set of ligand atoms, the atom state data for the ligand comprises features characterizing: (i) a 3D spatial position of the ligand atom, and (ii) one or more of: a partial charge of the ligand atom, a hybridization state of the ligand atom, or an elemental type of the ligand atom.

Embodiment 82 is the method of embodiment 80 or embodiment 81, wherein generating respective atom state data for each atom in the protein and for each ligand atom in the set of ligand atoms comprises, for one or more atoms:
stochastically sampling the atom state data for the atom.

Embodiment 83 is the method of embodiment 82, wherein for one or more atoms, stochastically sampling the atom state data for the atom comprises, for each of one or more continuous features of the atom:
stochastically sampling a value of the continuous feature of the atom from a probability distribution; and
including the stochastically sampled value of the continuous feature of the atom in the atom state data for the atom.

Embodiment 84 is the method of embodiment 83, wherein the one or more continuous features of the atom comprise respective features defining one or more of: a 3D spatial position of the atom or a partial charge of the atom.

Embodiment 85 is the method of any one of embodiments 82-84, wherein for one or more atoms, stochastically sampling the atom state data for the atom comprises, for each of one or more categorical features of the atom:
stochastically sampling a distribution over possible values of the categorical feature from a probability distribution; and
including the stochastically sampled distribution over possible values of the categorical feature of the atom in the atom state data for the atom.

Embodiment 86 is the method of embodiment 85, wherein the one or more categorical features of the atom comprise respective features defining one or more of: a hybridization state of the atom or an elemental type of the atom.

Embodiment 87 is the method of any one of embodiments 80-86, wherein generating the predicted ligand data based on the atom state data after a final time step in the sequence of time steps:
selecting a subset of the ligand atoms in the set of ligand atoms for inclusion in the ligand, wherein fewer than all of the ligand atoms in the set of ligand atoms are selected for inclusion in the ligand; and
filtering the set of ligand atoms to remove any ligand atom that is not selected for inclusion in the ligand.

Embodiment 88 is the method of embodiment 87, wherein selecting a subset of the ligand atoms in the set of ligand atoms for inclusion in the ligand comprises, for each ligand atom:
selecting the ligand atom for inclusion in the ligand only if a 3D spatial position of the ligand atom, as defined by the atom state data for the ligand atom, is at least a threshold distance from a predefined throw-away position;
wherein the generative model has been trained to move respective 3D spatial positions of ligand atoms that are not included in the ligand to the throw-away position.

Embodiment 89 is the method of embodiment 88, wherein generating the predicted ligand data based on the atom state data after the final time step in the sequence of time steps comprises, for each ligand atom in the set of ligand atoms:
determining a respective value of each of one or more continuous features of the ligand atom from the atom state data for the ligand atom, comprising, for each continuous feature:
   extracting a value of the continuous feature from one or more corresponding dimensions of the atom state data for the ligand atom.

Embodiment 90 is the method of any one of embodiments 88-89, wherein generating the predicted ligand data based on the atom state data after the final time step in the sequence of time steps comprises, for each ligand atom in the set of ligand atoms:
determining a respective value of each of one or more categorical features of the ligand atom from the atom state data for the ligand atom, comprising, for each categorical feature:
   extracting a distribution over possible values of the categorical feature from a plurality of corresponding dimensions of the atom state data for the ligand atom; and
   determining the value of the categorical feature based on the distribution over possible values of the categorical feature.

Embodiment 91 is the method of embodiment 90, wherein for one or more categorical features of the ligand atom, determining the value of the categorical feature based on the distribution over possible values of the categorical feature comprises:
stochastically sampling the value of the categorical feature from the distribution over possible values of the categorical feature; or
setting the value of the categorical feature equal to a possible value of the categorical feature having a highest score under the distribution over possible values of the categorical feature.

Embodiment 92 is the method of any one of embodiments 80-91, wherein denoising the atom state data over the sequence of time steps using the denoising neural network further comprises, at each of one or more time steps in the sequence of time steps:
processing at least some of the current atom state data using a ligand property prediction neural network to generate a predicted value of a ligand property of a ligand characterized by the current atom state data; and
determining gradients of a conditioning objective function with respect to at least some of the current atom state data, wherein the conditioning objective function measures a discrepancy between: (i) the predicted value of the ligand property, and (ii) a target value of the ligand property;
wherein generating atom state data of each atom in the protein and of each atom in the set of ligand atoms at the next time step using the denoising output comprises:
   combining the gradients of the conditioning objective function with the denoising output.

Embodiment 93 is the method of embodiment 92, wherein the ligand property prediction neural network processes the current atom state data for the atoms in the protein and for the ligand atoms in the set of ligand atoms.

Embodiment 94 is the method of any one of embodiments 92-93, wherein the ligand property prediction neural network generates a predicted value of a binding affinity of the ligand for the protein.

Embodiment 95 is the method of any one of embodiments 71-94, wherein the ligand design neural network is further configured to:
obtain a respective atom embedding for each atom in the ligand; and
generate, using a bond prediction machine learning model and based on the atom embeddings of the atoms in the ligand, bond data that defines, for each pair of atoms in the ligand, whether the pair of atoms are bonded.

Embodiment 96 is the method of embodiment 95, wherein obtaining the respective atom embedding for each atom in the ligand comprises generating the respective atom embedding for each atom in the ligand as an intermediate output of the ligand design neural network.

Embodiment 97 is the method of embodiment 96, wherein the generative model is a generative diffusion model comprising a denoising neural network; and
wherein for each atom in the ligand, the atom embedding of the atom is generated as an intermediate output of the denoising neural network at a final time step in a sequence of denoising time steps.

Embodiment 98 is the method of any one of embodiments 95-97, wherein generating, using the bond prediction machine learning model and based on the atom embeddings of the atoms in the ligand, the bond data comprises:
processing a one-dimensional (1D) sequence of atom embeddings of the atoms in the ligand to generate a two-dimensional (2D) array of pair embeddings; and
processing the 2D array of pair embeddings using the bond prediction machine learning model to generate the bond data.

Embodiment 99 is the method of any one of embodiments 95-98, wherein generating, using the bond prediction machine learning model and based on the atom embeddings of the atoms in the ligand, the bond data comprises:
generating, using the bond prediction machine learning model and based on (i) the atom embeddings of the atoms in the ligand and (ii) data defining a respective 3D spatial position of each atom in the ligand, the bond data.

Embodiment 100 is the method of any one of embodiments 1-99, wherein the molecule data comprises data defining each of one or more amino acid sequences of a first protein.

Embodiment 101 is the method of embodiment 100, wherein the first protein comprises an enzyme, receptor, or signaling protein that has been identified as being involved in a disease process.

Embodiment 102 is the method of any one of embodiments 1-101, wherein the molecule data comprises data defining each of one or more amino acid sequences of a second protein.

Embodiment 103 is the method of any one of embodiments 1-102, wherein the molecule data comprises a text string defining a chemical structure of a ligand.

Embodiment 104 is the method of embodiment 103, wherein the ligand is a small molecule.

Embodiment 105 is the method of any one of embodiments 1-104, wherein the molecule data comprises data defining one or more of: an amino acid sequence of a protein; a multiple sequence alignment (MSA) for a protein; a respective structure of each of one or more template proteins; a representation of a respective chemical structure of each of one or more ligands.

Embodiment 106 is the method of any one of embodiments 1-105, wherein the molecule data comprises data characterizing at least 6000 amino acid residues.

Embodiment 107 is a method performed by one or more computers, the method comprising:
obtaining data defining: (i) a protein, and (ii) a set of candidate ligands;
generating, using the method of any one of embodiments 16-40 and for each ligand of the set of ligands, a respective binding affinity score for the ligand that defines a predicted binding affinity of the protein and the ligand; and
determining a ranking of the set of candidate ligands based on the binding affinity scores for the ligands.

Embodiment 108 is the method of embodiment 107, further comprising:
selecting one or more candidate ligands from the set of candidate ligands based on the ranking for physically synthesis; and optionally
physically synthesizing the selected candidate ligands.

Embodiment 109 is the method of embodiment 108, further comprising, for each of the selected candidate ligands, performing experiments using physically synthesized instances of the candidate ligand to determine one or more of: an absorption of the candidate ligand, a distribution of the candidate ligand, a metabolism of the candidate ligand, or an excretion of the candidate ligand.

Embodiment 110 is the method of any one of embodiments 107-109, wherein the ranking of the candidate ligands ranks the candidate ligands from highest predicted binding affinity for the protein to lowest predicted binding affinity for the protein.

Embodiment 111 is a method performed by one or more computers, the method comprising:
obtaining data defining: (i) a set of candidate proteins, and (ii) a ligand;
generating, using the method of any one of embodiments 16-40 and for each candidate protein of the set of candidate proteins, a respective binding affinity score for the candidate protein that defines a predicted binding affinity of the candidate protein and the ligand; and
determining a ranking of the set of candidate proteins based on the binding affinity scores.

Embodiment 112 is the method of embodiment 111, further comprising:
selecting one or more candidate proteins from the set of candidate proteins based on the ranking for physically synthesis; and optionally
physically synthesizing the selected candidate proteins.

Embodiment 113 is the method of any one of embodiments 111-112, wherein the ranking of the candidate proteins ranks the candidate proteins from highest predicted binding affinity for the ligand to lowest predicted binding affinity for the ligand.

Embodiment 114 is a method performed by one or more computers, the method comprising:
obtaining a network input that characterizes a protein and one or more ligands;
generating, using the method of any one of embodiments 41-70, a predicted joint three-dimensional (3D) structure of the protein and the one or more ligands.

Embodiment 115 is the method of embodiment 115, further comprising:
selecting one or more of the ligands to be physically synthesized based at least in part on the predicted joint 3D structure of the protein and the one or more ligands; and optionally
physically synthesizing the selected ligands.

Embodiment 116 is the method of embodiment 114 or embodiment 115, further comprising:
selecting the protein to be physically synthesized based at least in part on the predicted joint 3D structure of the protein and the one or more ligands; and optionally
physically synthesizing the protein.

Embodiment 117 is a method comprising:
generating, for each ligand in a collection of ligands, a respective predicted joint 3D structure of the ligand and a protein using the method of any one of embodiments 41-70;
determining, for each ligand in the collection of ligands, a respective predicted binding affinity of the ligand for the protein based on the predicted joint 3D structure of the ligand and the protein; and
selecting one or more ligands in the collection of ligands for physical synthesis based at least in part on the predicted binding affinities.

Embodiment 118 is the method of embodiment 117, further comprising physically synthesizing the one or more selected ligands.

Embodiment 119 is a method comprising:
generating, for each protein in a collection of proteins, a respective predicted joint 3D structure of the protein and a ligand using the method of any one of embodiments 41-70;
determining, for each protein in the collection of proteins, a respective predicted binding affinity of the ligand for the protein based on the predicted joint 3D structure of the ligand and the protein; and
selecting one or more proteins in the collection of proteins for physical synthesis based at least in part on the predicted binding affinities.

Embodiment 120 is the method of embodiment 119, further comprising physically synthesizing the one or more selected proteins.

Embodiment 121 is a method performed by one or more computers for computationally designing a ligand for binding to a protein, the method comprising:
obtaining protein data characterizing at least a portion of a protein;
generating, using the method of any one of embodiments 71-99, predicted ligand data defining a predicted ligand that is predicted to bind to the protein; and
providing the predicted ligand data defining the predicted ligand.

Embodiment 122 is the method of embodiment 121, further comprising physically synthesizing the predicted ligand.

Embodiment 123 is a method comprising:
generating a collection of ligands for a protein using the method of any one of embodiments 71-99;
determining, for each ligand in the collection of ligands, one or more respective properties of the ligand; and
selecting one or more ligands in the collection of ligands for physical synthesis based at least in part on the properties of the ligands.

Embodiment 124 is the method of embodiment 123, further comprising physically synthesizing the one or more selected ligands.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

What is claimed is:

1. A method performed by one or more computers, the method comprising:
obtaining molecule data characterizing a molecule;
processing a network input comprising the molecule data using an embedding neural network to generate a molecule embedding representing the molecule;
processing the molecule embedding representing the molecule using a prediction machine learning model to generate an output prediction characterizing the molecule;
wherein the embedding neural network has been jointly trained along with a plurality of prediction neural networks that are each configured to perform a respective prediction task; and
wherein the plurality of prediction neural networks comprise a ligand design neural network that is configured to perform a ligand design task by operations comprising:
receiving an input molecule embedding that represents at least a portion of a protein and that is generated by the embedding neural network; and
processing the input molecule embedding to generate predicted ligand data defining a predicted ligand that is predicted to bind to the protein.

2. The method of claim 1, wherein:
the embedding neural network comprises one or more molecule embedding neural networks; and
the embedding neural network is configured to process molecule data characterizing a first molecule and a second molecule by performing operations comprising:
processing the molecule data characterizing the first molecule and the second molecule using the one or more molecule embedding neural networks to generate a molecule embedding of the first molecule;
processing the molecule data characterizing the first molecule and the second molecule using the one or more molecule embedding neural networks to generate a molecule embedding of the second molecule; and
processing the molecule embedding of the first molecule and the molecule embedding of the second molecule to generate a molecule embedding characterizing the first molecule and the second molecule.

3. The method of claim 2, wherein:
the molecule embedding of the first molecule comprises a respective component embedding for each of one or more molecular components of the first molecule;
the molecule embedding of the second molecule comprises a respective component embedding for each of one or more molecular components of the second molecule;
processing the molecule embedding of the first molecule and the molecule embedding of the second molecule to generate the molecule embedding characterizing the first molecule and the second molecule comprises:
generating data defining a 1D sequence of component embeddings by concatenating: (i) the component embeddings for the molecular components of the first molecule, and (ii) the component embeddings for the molecular components of the second molecule;
wherein the molecule embedding characterizing the first molecule and the second molecule is derived from the 1D sequence of component embeddings.

4. The method of claim 3, wherein processing the molecule embedding of the first molecule and the molecule embedding of the second molecule to generate the molecule embedding characterizing the first molecule and the second molecule further comprises:
transforming the 1D sequence of component embeddings into a two-dimensional (2D) array of embeddings;
wherein the molecule embedding characterizing the first molecule and the second molecule is derived from the 2D array of embeddings.

5. The method of claim 3, wherein the 1D sequence of component embeddings comprises a respective atom embedding for each of a plurality of atoms in the first molecule or the second molecule.

6. The method of claim 5, wherein 2D array of embeddings comprises a plurality of atom—atom embeddings that are each derived from a respective pair of atom embeddings within the 1D sequence of component embeddings.

7. The method of claim 3, wherein the 1D sequence of component embeddings comprises a respective amino acid embedding for each of a plurality of amino acids in the first molecule or the second molecule.

8. The method of claim 7, wherein the 2D array of embeddings comprises a plurality of amino acid—amino acid embeddings that are each derived from a respective pair of amino acid embeddings within the 1D sequence of component embeddings.

9. The method of claim 7, wherein the 2D array of embeddings comprises a plurality of amino acid—atom embeddings that are each derived from: (i) a respective atom embedding within the 1D sequence of component embeddings, and (ii) a respective amino acid embedding within the 1D sequence of component embeddings.

10. The method claim 4, wherein transforming the 1D sequence of component embeddings into the 2D array of embeddings comprises:
applying an outer product operation to the 1D sequence of component embeddings; or
applying a 2D concatenation operation to the 1D sequence of component embeddings.

11. The method of claim 4, wherein the embedding neural network further comprises a fusion neural network; and
wherein processing the molecule embedding of the first molecule and the molecule embedding of the second molecule to generate the molecule embedding characterizing the first molecule and the second molecule further comprises:
processing the 2D array of embeddings using the fusion neural network to generate an updated 2D array of embeddings;
wherein the updated 2D array of embeddings defines the molecule embedding characterizing the first molecule and the second molecule.

12. The method of claim 11, wherein the fusion neural network comprises a sequence of self-attention blocks, wherein each self-attention block is configured to perform operations comprising:
apply one or more self-attention operations to an input 2D array of embeddings to update the input 2D array of embeddings.

13. The method of claim 12, wherein for one or more of the self-attention blocks, the self-attention operations comprise one or more row-wise self-attention operations.

14. The method of claim 12, wherein for one or more of the self-attention blocks, the self-attention operations comprise one or more column-wise self-attention operations.

15. The method of claim 12, wherein for one or more of the self-attention blocks, the self-attention operations comprise one or more triangle self-attention operations.

16. The method of claim 1, wherein the plurality of prediction neural networks comprises a property prediction neural network that is configured to:
receive an input molecule embedding that represents a first molecule and a second molecule; and
generate a property score that defines a predicted joint property of the first molecule and the second molecule using the input molecule embedding.

17. The method of claim 16, wherein the property prediction neural network and the embedding neural network have been jointly trained on a plurality of training examples, wherein each training example comprises: (i) a training input that characterizes a first molecule and a second molecule for the training example, and (ii) a target property score that defines a property of the first molecule and the second molecule for the training example.

18. The method of claim 17, wherein the joint training of the property prediction neural network and the embedding neural network on the plurality of training examples comprises, for each training example:
processing the training input of the training example using the embedding neural network to generate a molecule embedding for the training example representing the first molecule and the second molecule for the training example;
processing the molecule embedding for the training example using the property prediction neural network to generate a predicted property score for the first molecule and the second molecule for the training example; and
backpropagating gradients of an objective function through the property prediction neural network and into the embedding neural network, wherein the objective function measures a discrepancy between: (i) the target property score specified by the training example, and (ii) the predicted property score generated by the embedding neural network and the property prediction neural network for the training example.

19. A system comprising:
one or more computers; and
one or more storage devices communicatively coupled to the one or more computers, wherein the one or more storage devices store instructions that, when executed by the one or more computers, cause the one or more computers to perform operations comprising:
obtaining molecule data characterizing a molecule;
processing a network input comprising the molecule data using an embedding neural network to generate a molecule embedding representing the molecule;
processing the molecule embedding representing the molecule using a prediction machine learning model to generate an output prediction characterizing the molecule;
wherein the embedding neural network has been jointly trained along with a plurality of prediction neural networks that are each configured to perform a respective prediction task; and
wherein the plurality of prediction neural networks comprise a ligand design neural network that is configured to perform a ligand design task by operations comprising:
receiving an input molecule embedding that represents at least a portion of a protein and that is generated by the embedding neural network; and
processing the input molecule embedding to generate predicted ligand data defining a predicted ligand that is predicted to bind to the protein.

20. One or more non-transitory computer storage media storing instructions that when executed by one or more computers cause the one or more computers to perform operations comprising:
obtaining molecule data characterizing a molecule;
processing a network input comprising the molecule data using an embedding neural network to generate a molecule embedding representing the molecule;
processing the molecule embedding representing the molecule using a prediction machine learning model to generate an output prediction characterizing the molecule;
wherein the embedding neural network has been jointly trained along with a plurality of prediction neural networks that are each configured to perform a respective prediction task; and
wherein the plurality of prediction neural networks comprise a ligand design neural network that is configured to perform a ligand design task by operations comprising:
receiving an input molecule embedding that represents at least a portion of a protein and that is generated by the embedding neural network; and
processing the input molecule embedding to generate predicted ligand data defining a predicted ligand that is predicted to bind to the protein.
